SEQUENCE LISTING (134 sequences)

SEQ ID NO: 1 - protein, 122 aa, Artificial Sequence
  heavy chain variable region
QVQLQESGPGLVRPSETLSLTCTVSGGSISSYSWSWIRQPPGKGLEWIGYIDYSGSTNYNPSLKSRVTISVDTSKTQFSLKLSSVSAADTAVYYCAKDLLYKWNYVEGFDIWGQGTTVTVSS

SEQ ID NO: 2 - protein, 122 aa, Artificial Sequence
  heavy chain variable region
QVQLQESGPGLVKPSETLSLTCTVSGGSISSYSWSWIRQPPGKGLEWIGYIDYSGTTNFNPSLKSRVTISVDTSKTQFSLKLSSVSAADTAVYYCAKDLLYKWNYVEGFDIWGQGTMVTVSS

SEQ ID NO: 3 - protein, 118 aa, Artificial Sequence
  heavy chain variable region
EVQLVESGGGVVQPGRSLRLSCAASGFTFSSYDTHWVRQAPGKGLEWVAVISYDGSNKYYADSVKGRFTISRDNSKNTLYLQMNSLRAEDTAMYYCARERGWDVFDIWGQGTLVTVSS

SEQ ID NO: 4 - protein, 121 aa, Artificial Sequence
  heavy chain variable region
QVQLVQSGAEVKKPGSSVKVSCKASGGTFSKYVVSWVRQAPGQGFDWMGGIIPMFGTANYAQMFQGRVTITADKSTSTVNMELSSLRSEDTAVYYCVRDKAVAGLYYFDSWGQGTLVTVSS

SEQ ID NO: 5 - protein, 117 aa, Artificial Sequence
  heavy chain variable region
QVQLQESGPGLVKPSETLSLTCTVSDDSISDYYWSWIRQPPGKGLEWIGYIYYSGNTKYNPSLKNRVTISVDTSKSQFSLKLTSVTAADTAVYYCARIPLTGEFDYWAQGTLVTVSS

SEQ ID NO: 6 - protein, 119 aa, Artificial Sequence
  heavy chain variable region
EVQLVESGGGVVQPGRSLRLSCAASGFTFSSYGMHWVRQAPGKGLEWVAVISYHGSDKYYADSVKGRFTISRDNSKNTLYLQMNSLRAEDTAVYYCARDGDNWDVFDIWGQGTLVTVSS

SEQ ID NO: 7 - protein, 121 aa, Artificial Sequence
  heavy chain variable region
QVQLVQSGAEVKKPGSSVKVSCKASGDTFSTYAINWIRQAPGQGLEWMGGIIPIFGTAYYAQEFQDRVTITADKSTSTGYMEMSSLISEDTAVYYCARERELGALYAFDIWGQGTMVTVSS

SEQ ID NO: 8 - protein, 121 aa, Artificial Sequence
  heavy chain variable region
EVQLVQSGAEVKKPGSSVKVSCKASGGTFSSHAISWVRQVPGQGLEWMGGIIPLFDTAKNAQKFQGRVTITADKSTSTAYMELSSLRSEDTAVYYCARDRETGTLYYFDYWGQGTLVTVSS

SEQ ID NO: 9 - protein, 119 aa, Artificial Sequence
  heavy chain variable region
EVQLVQSGSELKKPGASVKVSCKASGYTFTTNALNWVRQAPGQGLEWMGWINTHTGNPTYAQGFIGRFVFSLDTSVSTAYLQIRSLKAEDTAVYYCAREPNWGVYFDYWGQGTLVTVSS

SEQ ID NO: 10 - protein, 125 aa, Artificial Sequence
  heavy chain variable region
QVQLVQSGAEVKRPGASVKVSCKVSGYTLTELSMHWVRQAPGKGLEWMGGSDPEHGETVDAQKFQGRVTMTEDTSTDTAYMELSSLRSEDTAVYYCTTGGTYYYGSGSYYTLDFWGQGTLVTVSS

SEQ ID NO: 11 - protein, 121 aa, Artificial Sequence
  heavy chain variable region
QVQLVQSGAEVKKPGSSVKVSCKASGGTFSNFAFSWVRQAPGQGLEWMGGIIPMFDTAKYAQKFQGRVTIIADKSTNTAYMDLNSLRSEDTAVYYCVRDRAIGTLYYFDYWGQGTLVTVSS

SEQ ID NO: 12 - protein, 121 aa, Artificial Sequence
  heavy chain variable region
QVQLVQSGAEVRKPGSSVMVSCKASGGTFNTYAINWVRQAPGQGLEWMGGIIPIFGTPYYGQRFQGRVTITADKSTNTVFMELSSLRSEDTAMYFCARERDIGSLYYFDSWGQGTLVTVSS

SEQ ID NO: 13 - protein, 121 aa, Artificial Sequence
  heavy chain variable region
QVQLVQSGAEVRKPGSSVMVSCKASGGTFSTYAINWLRQAPGQGLEWMGGIIPIFGTPYYGQRFQGRVTITADKSTNTVFMELSSLRSEDTAIYYCARDRDSGGLYYFDSWGQGTLVTVSS

SEQ ID NO: 14 - protein, 121 aa, Artificial Sequence
  heavy chain variable region
QVQLVQSGAEVKKPGSSVKVSCKTSGGTFSNYAFSWVRQAPGQGLEWMGGIIPIFGSTNYAQSFQGRVTITADKSTSTAYMELSSLRSEDTAVYYCARDREMGTLYFFDQWGQGTTVTVSS

SEQ ID NO: 15 - protein, 119 aa, Artificial Sequence
  heavy chain variable region
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARDGSGWDDFDYWGQGTLVTVSS

SEQ ID NO: 16 - protein, 121 aa, Artificial Sequence
  heavy chain variable region
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARGSILAAQMWGDIWGQGTLVTVSS

SEQ ID NO: 17 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCARERGWDVFDIWGQGTTVTVSS

SEQ ID NO: 18 - protein, 126 aa, Artificial Sequence
  heavy chain variable region
EVQLVESGGGLVQPGGSLRLSCAASGFTFSSYAMSWVRQAPGKGLEWVSSISGGGVSTFYADSVKGRFTISRDNSKNTLYLQMNSLRAEDTAVYYCAIVPAAATPSGTYYWIFDLWGRGTLVTVSS

SEQ ID NO: 19 - protein, 125 aa, Artificial Sequence
  heavy chain variable region
EVQLVESGGGLVQPGGSLRLSCAASGFTFSSYAMNWVRQAPGKGLEWVSTISGSGVSTYYADSVKGRFTISRDNSKNTLYLQMNTLRAEDTAVYYCAKDRGYDYSGSYHNWFDPWGQGTLVTVSS

SEQ ID NO: 20 - protein, 121 aa, Artificial Sequence
  heavy chain variable region
QVQLVQSGAEVKKPGSSVKVSCKASGGTFSTYAISWVRQAPGQGLEWMGWIIPIFGTGNYAQKFQGRVTITADKSTSTAYMELRSLRSEDTAVHYCARHDYTNTVDAFDIWGQGTMVTVSS

SEQ ID NO: 21 - protein, 121 aa, Artificial Sequence
  heavy chain variable region
QVQLVQSGAEVKKPGSSVKVSCKASGDTFRSYGITWVRQAPGQGLEWMGGIIPIFGTTNYAQKFQGRVTITADKSTSTVYMELSSLRSEDTAVYYCARRRGYSNPHWLDPWGQGTLVTVSS

SEQ ID NO: 22 - protein, 119 aa, Artificial Sequence
  heavy chain variable region
QVQLVQSGAEVKKPGSSVKVSCKASGGTFSTYGILWVRQAPGQGLEWMGGIIPIFGTANYAQKFQGRVTITADISTSTAYMELSSLRSEDTAVYYCARGGGNYYEFVYWGQGTLVTVSS

SEQ ID NO: 23 - protein, 121 aa, Artificial Sequence
  heavy chain variable region
EVQLVQSGAEVKKPGSSVKVSCKASGGTFSTYAISWVRQAPGQGLEWMGWIIPIFDTGNYAQKIQGRVTITADKSTSTAYMELTSLRSEDTAVYYCARHDYTNTVDAFDIWGQGTMVTVSS

SEQ ID NO: 24 - protein, 121 aa, Artificial Sequence
  heavy chain variable region
EVQLVQSGAEVKKPGSSVKVSCKASGGTFSTYAISWVRQAPGQGLEWMGWIIPIFDTGNYAQKFQGRVTITADKSTSTAYMELTSLRSEDTAVYYCARHDYTNTVDAFDIWGQGTMVTVSS

SEQ ID NO: 25 - protein, 118 aa, Artificial Sequence
  heavy chain variable region
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 26 - protein, 107 aa, Artificial Sequence [sequence omitted]

SEQ ID NO: 27 - protein, 120 aa, Artificial Sequence
  heavy chain variable region
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 28 - protein, 107 aa, Artificial Sequence
  light chain variable region
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 29 - protein, 125 aa, Artificial Sequence
  heavy chain variable region
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSS

SEQ ID NO: 30 - protein, 107 aa, Artificial Sequence
  light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 31 - protein, 6 aa, Artificial Sequence
  Light chain CDR1
QSISSY

SEQ ID NO: 32 - protein, 3 aa, Artificial Sequence
  Light chain CDR2
AAS

SEQ ID NO: 33 - protein, 9 aa, Artificial Sequence
  Light chain CDR3
QQSYSTPPT

SEQ ID NO: 34 - protein, 108 aa, Artificial Sequence
  light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 35 - protein, 107 aa, Artificial Sequence
  light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 36 - protein, 108 aa, Artificial Sequence
  light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 37 - protein, 108 aa, Artificial Sequence
  light chain variable region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 38 - protein, 95 aa, Artificial Sequence
  VK1-39
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 39 - protein, 95 aa, Artificial Sequence
  VK3-15
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 40 - protein, 96 aa, Artificial Sequence
  VK3-20
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 41 - protein, 96 aa, Artificial Sequence
  VK3-21
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 42 - protein, 98 aa, Artificial Sequence
  CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 43 - protein, 107 aa, Artificial Sequence [sequence omitted]

SEQ ID NO: 44 - protein, 15 aa, Artificial Sequence
  amino acid sequence
EPKSCDKTHTCPPCP

SEQ ID NO: 45 - protein, 110 aa, Artificial Sequence
  CH2
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 46 - protein, 4 aa, Artificial Sequence
  intentionally skipped sequence
  misc_feature (1)..(4): Xaa can be any naturally occurring amino acid
XXXX

SEQ ID NO: 47 - protein, 107 aa, Artificial Sequence
  CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 4 aa, Artificial Sequence
  intentionally skipped sequence
  misc_feature (1)..(4): Xaa can be any naturally occurring amino acid
XXXX

SEQ ID NO: 49 - protein, 4 aa, Artificial Sequence
  intentionally skipped sequence
  misc_feature (1)..(4): Xaa can be any naturally occurring amino acid
XXXX

SEQ ID NO: 50 - protein, 107 aa, Artificial Sequence
  CH3
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 107 aa, Artificial Sequence
  CH3
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 117 aa, Artificial Sequence
  heavy chain variable region
QVQLQESGPGLVKPSETLSLTCTVSDDSISDYYWSWIRQPPGKGLEWIGYIYYSGNTKYNPSLKNRVTISVDTSKSQFSLKLTSVTAADTAVYYCARIPLTGDFDFWAQGTLVTVSS

SEQ ID NO: 53 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCAKERGWDVFDLWGQGTTVTVSS

SEQ ID NO: 54 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCAKERGWDVFDVWGQGTTVTVSS

SEQ ID NO: 55 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCAREKGWDVFDLWGQGTTVTVSS

SEQ ID NO: 56 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCAREKGWDVFDVWGQGTTVTVSS

SEQ ID NO: 57 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCARERGWDIFDVWGQGTTVTVSS

SEQ ID NO: 58 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCARERGWDLFDLWGQGTTVTVSS

SEQ ID NO: 59 - protein, 118 aa, Artificial Sequence
  heavy chain variable region
QVQLVQSGGGVVQPGRSLRLSCAASGFTFSSYDTHWVRQAPGKGLEWVAVISYDGSNKYYADSVKGRFTISRDNSKNTLYLHMNSLRAEDTAMYYCARERGWDLFDVWGQGTTVTVSS

SEQ ID NO: 60 - protein, 5 aa, Artificial Sequence
  HCDR1
SYSWS

SEQ ID NO: 61 - protein, 16 aa, Artificial Sequence
  HCDR2
YIDYSGSTNYNPSLKS

SEQ ID NO: 62 - protein, 14 aa, Artificial Sequence
  HCDR3
DLLYKWNYVEGFDI

SEQ ID NO: 63 - protein, 16 aa, Artificial Sequence
  HCDR2
YIDYSGTTNFNPSLKS

SEQ ID NO: 64 - protein, 5 aa, Artificial Sequence
  HCDR1
SYDTH

SEQ ID NO: 65 - protein, 17 aa, Artificial Sequence
  HCDR2
VISYDGSNKYYADSVKG

SEQ ID NO: 66 - protein, 9 aa, Artificial Sequence
  HCDR3
ERGWDVFDI

SEQ ID NO: 67 - protein, 5 aa, Artificial Sequence
  HCDR1
KYVVS

SEQ ID NO: 68 - protein, 17 aa, Artificial Sequence
  HCDR2
GIIPMFGTANYAQMFQG

SEQ ID NO: 69 - protein, 12 aa, Artificial Sequence
  HCDR3
DKAVAGLYYFDS

SEQ ID NO: 70 - protein, 5 aa, Artificial Sequence
  HCDR1
DYYWS

SEQ ID NO: 71 - protein, 16 aa, Artificial Sequence
  HCDR2
YIYYSGNTKYNPSLKN

SEQ ID NO: 72 - protein, 9 aa, Artificial Sequence
  HCDR3
IPLTGEFDY

SEQ ID NO: 73 - protein, 5 aa, Artificial Sequence
  HCDR1
SYGMH

SEQ ID NO: 74 - protein, 17 aa, Artificial Sequence
  HCDR2
VISYHGSDKYYADSVKG

SEQ ID NO: 75 - protein, 10 aa, Artificial Sequence
  HCDR3
DGDNWDVFDI

SEQ ID NO: 76 - protein, 5 aa, Artificial Sequence
  HCDR1
TYAIN

SEQ ID NO: 77 - protein, 17 aa, Artificial Sequence
  HCDR2
GIIPIFGTAYYAQEFQD

SEQ ID NO: 78 - protein, 12 aa, Artificial Sequence
  HCDR3
ERELGALYAFDI

SEQ ID NO: 79 - protein, 5 aa, Artificial Sequence
  HCDR1
SHAIS

SEQ ID NO: 80 - protein, 17 aa, Artificial Sequence
  HCDR2
GIIPLFDTAKNAQKFQG

SEQ ID NO: 81 - protein, 12 aa, Artificial Sequence
  HCDR3
DRETGTLYYFDY

SEQ ID NO: 82 - protein, 5 aa, Artificial Sequence
  HCDR1
TNALN

SEQ ID NO: 83 - protein, 17 aa, Artificial Sequence
  HCDR2
WINTHTGNPTYAQGFIG

SEQ ID NO: 84 - protein, 10 aa, Artificial Sequence
  HCDR3
EPNWGVYFDY

SEQ ID NO: 85 - protein, 5 aa, Artificial Sequence
  HCDR1
ELSMH

SEQ ID NO: 86 - protein, 17 aa, Artificial Sequence
  HCDR2
GSDPEHGETVDAQKFQG

SEQ ID NO: 87 - protein, 16 aa, Artificial Sequence
  HCDR3
GGTYYYGSGSYYTLDF

SEQ ID NO: 88 - protein, 5 aa, Artificial Sequence
  HCDR1
NFAFS

SEQ ID NO: 89 - protein, 17 aa, Artificial Sequence
  HCDR2
GIIPMFDTAKYAQKFQG

SEQ ID NO: 90 - protein, 12 aa, Artificial Sequence
  HCDR3
DRAIGTLYYFDY

SEQ ID NO: 91 - protein, 17 aa, Artificial Sequence
  HCDR2
GIIPIFGTPYYGQRFQG

SEQ ID NO: 92 - protein, 12 aa, Artificial Sequence
  HCDR3
ERDIGSLYYFDS

SEQ ID NO: 93 - protein, 12 aa, Artificial Sequence
  HCDR3
DRDSGGLYYFDS

SEQ ID NO: 94 - protein, 5 aa, Artificial Sequence
  HCDR1
NYAFS

SEQ ID NO: 95 - protein, 17 aa, Artificial Sequence
  HCDR2
GIIPIFGSTNYAQSFQG

SEQ ID NO: 96 - protein, 12 aa, Artificial Sequence
  HCDR3
DREMGTLYFFDQ

SEQ ID NO: 97 - protein, 5 aa, Artificial Sequence
  HCDR1
SYGIS

SEQ ID NO: 98 - protein, 17 aa, Artificial Sequence
  HCDR2
WISAYSGNTNYAQKLQG

SEQ ID NO: 99 - protein, 10 aa, Artificial Sequence
  HCDR3
DGSGWDDFDY

SEQ ID NO: 100 - protein, 12 aa, Artificial Sequence
  HCDR3
GSILAAQMWGDI

SEQ ID NO: 101 - protein, 5 aa, Artificial Sequence
  amino acid sequence
SYAMS

SEQ ID NO: 102 - protein, 17 aa, Artificial Sequence
  amino acid sequence
SISGGGVSTFYADSVKG

SEQ ID NO: 103 - protein, 17 aa, Artificial Sequence
  amino acid sequence
VPAAATPSGTYYWIFDL

SEQ ID NO: 104 - protein, 5 aa, Artificial Sequence
  amino acid sequence
SYAMN

SEQ ID NO: 105 - protein, 17 aa, Artificial Sequence
  amino acid sequence
TISGSGVSTYYADSVKG

SEQ ID NO: 106 - protein, 16 aa, Artificial Sequence
  amino acid sequence
DRGYDYSGSYHNWFDP

SEQ ID NO: 107 - protein, 5 aa, Artificial Sequence
  HCDR1
TYAIS

SEQ ID NO: 108 - protein, 17 aa, Artificial Sequence
  HCDR2
WIIPIFGTGNYAQKFQG

SEQ ID NO: 109 - protein, 12 aa, Artificial Sequence
  HCDR3
HDYTNTVDAFDI

SEQ ID NO: 110 - protein, 5 aa, Artificial Sequence
  HCDR1
SYGIT

SEQ ID NO: 111 - protein, 17 aa, Artificial Sequence
  HCDR2
GIIPIFGTTNYAQKFQG

SEQ ID NO: 112 - protein, 12 aa, Artificial Sequence
  HCDR3
RRGYSNPHWLDP

SEQ ID NO: 113 - protein, 5 aa, Artificial Sequence
  HCDR1
TYGIL

SEQ ID NO: 114 - protein, 17 aa, Artificial Sequence
  HCDR2
GIIPIFGTANYAQKFQG

SEQ ID NO: 115 - protein, 10 aa, Artificial Sequence
  HCDR3
GGGNYYEFVY

SEQ ID NO: 116 - protein, 17 aa, Artificial Sequence
  HCDR2
WIIPIFDTGNYAQKIQG

SEQ ID NO: 117 - protein, 17 aa, Artificial Sequence
  HCDR2
WIIPIFDTGNYAQKFQG

SEQ ID NO: 118 - protein, 9 aa, Artificial Sequence
  HCDR3
IPLTGDFDF

SEQ ID NO: 119 - protein, 9 aa, Artificial Sequence
  HCDR3
ERGWDVFDL

SEQ ID NO: 120 - protein, 9 aa, Artificial Sequence
  HCDR3
ERGWDVFDV

SEQ ID NO: 121 - protein, 9 aa, Artificial Sequence
  HCDR3
EKGWDVFDL

SEQ ID NO: 122 - protein, 9 aa, Artificial Sequence
  HCDR3
EKGWDVFDV

SEQ ID NO: 123 - protein, 9 aa, Artificial Sequence
  HCDR3
ERGWDIFDV

SEQ ID NO: 124 - protein, 9 aa, Artificial Sequence
  HCDR3
ERGWDLFDL

SEQ ID NO: 125 - protein, 9 aa, Artificial Sequence
  HCDR3
ERGWDLFDV

SEQ ID NO: 126 - protein, 5 aa, Artificial Sequence
  HCDR1
  VARIANT (2)..(2): can be Y or F
  VARIANT (3)..(3): can be Y or S
SXXWS

SEQ ID NO: 127 - protein, 16 aa, Artificial Sequence
  HCDR2
  VARIANT (3)..(3): can be Y or D
  VARIANT (7)..(7): can be S or T
  VARIANT (10)..(10): can be Y or F
  VARIANT (13)..(13): can be S or F
  VARIANT (16)..(16): can be S or I
YIXYSGXTNXNPXLKX

SEQ ID NO: 128 - protein, 14 aa, Artificial Sequence
  HCDR3
  VARIANT (1)..(1): can be D or H
XLLYKWNYVEGFDI

SEQ ID NO: 129 - protein, 5 aa, Artificial Sequence
  HCDR1
  VARIANT (1)..(1): can be S, N, or R
  VARIANT (3)..(3): can be G or D
  VARIANT (4)..(4): can be M, T or I
XYXXH

SEQ ID NO: 130 - protein, 17 aa, Artificial Sequence
  HCDR2
  VARIANT (7)..(7): can be S or N
  VARIANT (10)..(10): can be Y, F, or H
  VARIANT (12)..(12): can be A, E, or V
VISYDGXNKXYXDSVKG

SEQ ID NO: 131 - protein, 9 aa, Artificial Sequence
  HCDR3
  VARIANT (3)..(3): can be G or D
ERXWDVFDI

SEQ ID NO: 132 - protein, 5 aa, Artificial Sequence
  HCDR1
  VARIANT (1)..(1): can be S or N
  VARIANT (3)..(3): can be G or A
XYXMH

SEQ ID NO: 133 - protein, 17 aa, Artificial Sequence
  HCDR2
  VARIANT (5)..(5): can be D or H
  VARIANT (8)..(8): can be N or D
VISYXGSXKYYADSVKG

SEQ ID NO: 134 - protein, 10 aa, Artificial Sequence
  HCDR3
  VARIANT (7)..(7): can be V or A
DGDNWDXFDI

(19) United States
(12) Patent Application Publication
(10) Pub. No.: US 2024/0309115 A1
(43) Pub. Date: Sep. 19, 2024

(54) NOVEL MULTISPECIFIC ANTIBODIES

(71) Applicant: MERUS N.V., Utrecht (NL)

(72) Inventors: Simon Edward PLYTE, Utrecht (NL); Cornelis Adriaan DE KRUIF, Utrecht (NL)

(21) Appl. No.: 18/553,114

(22) PCT Filed: Mar. 30, 2022
(86) PCT No.: PCT/NL2022/050174

§ 371 Date: Sep. 28, 2023

(30) Foreign Application Priority Data
Mar. 31, 2021 (NL) 2027891

(51) Int. Cl. C07K 16/46; A61K 39/00; A61P 35/00; C12N 15/64

(52) U.S. Cl. CPC C07K 16/468; A61P 35/00; C12N 15/64; A61K 2039/505; C07K 2317/31; C07K 2317/522; C07K 2317/524; C07K 2317/526; C07K 2317/53; C07K 2317/565

(57) ABSTRACT

The present disclosure relates to a multispecific antibody comprising a binding domain that binds to LAG-3 and a binding domain that binds to PD-L1. Such multispecific antibody has comparable, or equal or higher, potency than a combination of LAG-3 and PD-L1 reference antibodies. Also provided is a method for treating a disease, in particular a disease associated with a suppressed immune system, such as cancer, with a multispecific antibody of the present disclosure. The present disclosure further relates to a vector and cell comprising nucleic acids encoding the heavy chain variable region of the LAG-3 and PD-L1 binding domains.

### FIELD

The present disclosure relates to the field of antibodies. In particular it relates to the field of therapeutic antibodies for the treatment of diseases involving aberrant cells. More in particular it relates to novel multispecific antibodies and variants thereof that bind to LAG-3 and a protein of the B7 family, in particular PD-L1.

### BACKGROUND

Cancer is still a major cause of death in the world, in spite of the many advances that have been made in the treatment of the disease and the increased knowledge of the molecular events that lead to cancer. Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases the tumors in the patients stop growing or temporarily shrink (referred to as remission) only to start proliferating again, sometimes more rapidly (referred to as relapse), and become increasingly more difficult to treat. Over the past years, the focus of cancer drug development has moved away from broadly cytotoxic chemotherapy to targeted cytostatic therapies with less toxicity. Treatment of advanced cancer with targeted therapies has been validated clinically in leukemia and some other cancers. However, in a majority of carcinomas, targeted approaches are still proving not effective enough to completely abolish cancer in the majority of the patients.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

A developing class of therapeutic antibodies are bispecific antibodies, which comprise two different binding sites that bind different antigens or different epitopes on the same antigen. Bispecific antibodies can be designed for several applications. Firstly, bispecific antibodies may provide greater tissue-specificity than a monospecific antibody. Several tumor-associated antigens are not only (over)expressed by tumor cells but are also expressed on normal, healthy cells. A bispecific antibody directed against two different tumor-associated antigens involved in a particular type of cancer can specifically target the antibody to the tumor site where the antibody induces tumor cell killing, thereby preventing binding to non-tumor cells expressing only one of the antigens and thus reducing off-site toxicity. Other mechanisms of action include for instance the engagement of immune cells to tumor cells, and the disruption of two signaling pathways required for tumor growth.

Immune checkpoint proteins, like for instance PD-1, PD-L1, CTLA-4, LAG-3, and TIM-3, are an interesting target for antibody therapy. To date, a number of monospecific antibodies targeting LAG-3 or PD-L1 have been described, as well as certain bispecific antibodies that bind LAG-3 and PD-L1. However, each of these bispecific antibodies has its own challenges in the production of an effective therapeutic drug. There thus remains a need for the development of novel, effective LAG-3×PD-L1 bispecific antibodies.

### SUMMARY

One of the objects of the present disclosure is to provide a new pharmaceutical agent for the treatment of human disease, in particular for the treatment of cancer. This object is met by the provision of a multispecific antibody comprising an anti-human LAG-3 binding domain and an anti-human PD-L1 binding domain.

In certain embodiments, the present disclosure provides a multispecific antibody comprising a binding domain that binds to LAG-3 and a binding domain that binds to PD-L1, or a variant thereof that maintains the binding specificity of the antibody, wherein the antibody or variant has at least comparable, or equal or higher, potency than a combination of reference antibodies. In certain embodiments, the combination of reference antibodies comprises a reference antibody comprising two heavy chain variable regions having an amino acid sequence as set forth in SEQ ID NO: 27 and two light chain variable regions having an amino acid sequence as set forth in SEQ ID NO: 28 (a relatlimab analog antibody), and a reference antibody comprising two heavy chain variable regions having an amino acid sequence as set forth in SEQ ID NO: 25 and two light chain variable regions having an amino acid sequence as set forth in SEQ ID NO: 26 (an atezolizumab analog antibody).

In certain embodiments, the present disclosure further provides a multispecific antibody, or variant thereof that maintains the binding specificity of the antibody, wherein the antibody or variant thereof comprises a binding domain that specifically binds to an extracellular domain of LAG-3 and a binding domain that specifically binds to an extracellular domain of a protein of the B7 family, wherein the LAG-3 binding domain comprises a heavy chain variable region comprising heavy chain CDR1 (HCDR1), heavy chain CDR2 (HCDR2), and heavy chain CDR3 (HCDR3), of a heavy chain variable region having an amino acid sequence as set forth in any one of SEQ ID NOs: 1-17 or SEQ ID NOs: 52-59.

In certain embodiments, the present disclosure further provides a pharmaceutical composition comprising an effective amount of the multispecific antibody or variant thereof as described herein.

In certain embodiments, the present disclosure further provides a multispecific antibody or variant thereof, or pharmaceutical composition, as described herein for use as a medicament.

In certain embodiments, the present disclosure further provides a multispecific antibody or variant thereof, or a pharmaceutical composition, as described herein, for use in the treatment of a disease associated with a suppressed immune system. In certain embodiments, the present disclosure provides a multispecific antibody or variant thereof, or a pharmaceutical composition, as described herein, for use in the treatment of cancer.

In certain embodiments, the present disclosure further provides a method for treating (a) a disease, or (b) a disease associated with a suppressed immune system, or c) cancer, comprising administering an effective amount of the multispecific antibody or variant thereof, or the pharmaceutical composition, as described herein, to an individual in need thereof.

In certain embodiments, the present disclosure further provides a vector comprising a nucleic acid sequence encoding a heavy chain variable region of a LAG-3 binding domain as described herein and a nucleic acid sequence encoding a heavy chain variable region of a PD-L1 binding domain as described herein.

In certain embodiments, the present disclosure further provides a cell comprising a nucleic acid sequence encoding a heavy chain variable region of a LAG-3 binding domain as described herein and a nucleic acid sequence encoding a heavy chain variable region of a PD-L1 binding domain as described herein

In certain embodiments, the present disclosure further provides a cell producing a multispecific antibody or variant thereof as described herein.

In certain embodiments, the present disclosure provides a method for producing a variant of a multispecific antibody as described herein, as well as a variant obtained by such method.

### DETAILED DESCRIPTION

In certain embodiments, the present disclosure provides a multispecific antibody, comprising a binding domain that binds to LAG-3 and a binding domain that binds to PD-L1, or a variant thereof that maintains the binding specificity of the antibody, wherein the antibody or variant has at least comparable, or equal or higher, potency than a combination of reference antibodies, wherein the combination of reference antibodies comprises a reference antibody comprising two heavy chain variable regions having an amino acid sequence as set forth in SEQ ID NO: 27 and two light chain variable regions having an amino acid sequence as set forth in SEQ ID NO: 28, and a reference antibody comprising two heavy chain variable regions having an amino acid sequence as set forth in SEQ ID NO: 25 and two light chain variable regions having an amino acid sequence as set forth in SEQ ID NO: 26.

LAG-3 is known under a number of different names such as Lymphocyte Activating 3; Lymphocyte-Activation Gene 3; CD223 Antigen; Protein FDC; CD223; LAG-3; or FDC. External Ids for LAG3 are: HGNC: 6476; Entrez Gene: 3902; Ensembl: ENSG00000089692; OMIM: 153337; and UniProtKB: P18627. LAG-3 is closely related to CD4. LAG-3 is located on the human chromosome 12 (12p13.32) adjacent to the CD4 gene, and its sequence is approximately 20% identical to CD4. The LAG-3 protein binds a nonholomorphic region of major histocompatibility complex 2 (MHC class II) with greater affinity than CD4. LAG-3 is one of the various immune-checkpoint receptors that are coordinately upregulated on both regulatory T cells (Tregs) and anergic T cells. LAG-3 can negatively regulate T cell proliferation, activation and homeostasis.

PD-L1 is a type 1 transmembrane protein that plays a role in suppressing an immune response during particular events such as pregnancy, tissue allografts, autoimmune disease and other disease states such as hepatitis. PD-L1 is expressed in various types of cancers, especially in NSCLC (Boland et al., 2013; Velcheti et al., 2014), melanoma, renal cell carcinoma, gastric cancer, hepatocellular as well as various leukemias and multiple myeloma (Bernstein et al., 2014; Thompson et al., 2005). PD-L1 is present in the cytoplasm and plasma membrane of cancer cells, but not all cancers or all cells within a tumor express PD-L1 (Dong et al., 2002). Multiple tumor microenvironment cells contribute to immune suppression by upregulating PD-L1 expression. This effect is called “adaptive immune resistance”, because the tumor protects itself by inducing PD-L1 in response to IFN-γ produced by activated T cells (Sharma et al., 2017). PD-L1 can also be regulated by oncogenes, this mechanism is known as inherent immune resistance (Akbay et al., 2013). Within the tumor microenvironment, PD-L1 is also expressed on myeloid cells and activated T cells (Tumeh et al., 2014). The expression of PD-L1 is induced by multiple proinflammatory molecules, including types I and II IFN-γ, TNF-α, LPS, GM-CSF and VEGF, as well as the cytokines IL-10 and IL-4, with IFN-γ being the most potent inducer (Sznol and Chen, 2013).

A multispecific antibody according to the present disclosure is an antibody, in any antibody format, that comprises at least two binding domains which have specificity for at least two different targets or epitopes. In certain embodiments, a multispecific antibody of the present disclosure is a bispecific antibody. In certain embodiments, a multispecific antibody of the present disclosure comprises an Fc region or a part thereof. In certain embodiments, a multispecific antibody of the present disclosure is an IgG1 antibody.

A “variant” of a multispecific antibody as described herein comprises a functional part, derivative, and/or analogue of the multispecific antibody. The variant may be a structural variant, including but not limited to a fragment of an antibody, such as for example a Fab fragment or a single-chain variable fragment (scFv). The variant may be a sequence variant. The variant may be a structural and sequence variant. The variant maintains the binding specificity, but not necessarily the binding affinity, of the antibody.

In certain embodiments, the LAG-3 and/or PD-L1 binding domain comprises at least a heavy chain variable region and a light chain variable region. The light chain variable region can be any suitable light chain variable region as described further herein. In certain embodiments, the light chain variable region preferably is a light chain variable region of a light chain that is capable of pairing with multiple heavy chains having different epitope specificities. Such light chain is also referred to in the art as a “common light chain”.

In certain embodiments, the multispecific antibody or variant thereof comprises a single binding domain that binds to LAG-3 and/or a single binding domain that binds to PD-L1. In other words, in certain embodiments the multispecific antibody or variant thereof is monovalent for binding to LAG-3 and/or monovalent for binding to PD-L1. In certain embodiments, the multispecific antibody or variant thereof is monovalent for binding to both LAG-3 and PD-L1.

Determining if a multispecific antibody or variant has a comparable, or equal or higher, potency than the combination of reference antibodies can be done by measuring the potency of both the multispecific antibody and combination of antibodies in the same type of study, using the same study conditions. Thus, in certain embodiments, the potency of the multispecific antibody or variant thereof is measured in the same type of study, using the same study conditions.

In certain embodiments, an at least comparable potency is a potency within a 5 fold range of the potency of the combination of reference antibodies, and includes a 5, 4, 3, or 2 fold, preferably a 3 or 2 fold, deviation from the potency of the combination of reference antibodies.

In the context of the present disclosure, “potency” refers to the functional activity of the multispecific antibody or variant thereof, which can be determined in in vitro or in in vivo studies.

In certain embodiments, the potency of the multispecific antibody or variant thereof is determined in an in vivo study, preferably in an in vivo mouse model, such as for instance a HuNSG™ mouse model bearing human MDA-231 tumors. In certain embodiments, the potency of the multispecific antibody or variant thereof is determined by measuring tumor volume reduction in such an in vivo mouse study. The tumor volume reduction induced by the multispecific antibodies as provided herein is determined with the method as described in Example 4.

In certain embodiments, a comparable potency is a tumor volume reduction within a 5 fold range of the tumor volume reduction of the combination of reference antibodies, and includes a 5, 4, 3, or 2 fold, preferably a 3 or 2 fold, deviation from the tumor volume reduction of the combination of reference antibodies.

In certain embodiments, the potency is determined in an in vitro study, such as for instance a blocking assay, including but not limited to a PD-1/LAG-3 or PD-L1/LAG-3 reporter assay, preferably a PD-1/LAG-3 reporter assay, such as for instance described in Example 2; or a T cell activation assay, including but not limited to a SEB assay, preferably a SEB assay such as for instance described in Example 2; an antigen recall assay; or an MLR assay. In certain embodiments, the potency of the multispecific antibody or variant thereof and the combination of reference antibodies is determined by measuring their potency in blocking ligand or receptor binding to LAG-3 and/or PD-L1, preferably ligand or receptor binding to LAG-3 and PD-L1. The potency in blocking ligand or receptor binding to LAG-3 and/or PD-L1 of the multispecific antibodies as provided herein is measured with the method as described in Example 2. In brief, the PD-1/LAG-3 reporter assay is performed using PD-1 and LAG-3 expressing Jurkat T cells as effector cells and PD-L1 expressing Raji cells as target cells. The PD-1 and LAG-3 effector cells are prepared and plated at 100,000 cells per well. Test and control IgG are added in a 6-step semi-log titration at equimolar amounts, followed by the Raji cells (25,000 per well). The T cells are activated by the addition of partially purified Staphylococcal enterotoxin D (ppSED, using a concentration that achieves a highest response with the positive assay control, such as 16.6 ng/mL in Example 2 or 150 ng/mL in Example 5). After 6 hours incubation at 37° C., luciferase reporter gene activity is determined.

In certain embodiments, a comparable potency is a potency in blocking ligand or receptor binding to LAG-3 and/or PD-L1, preferably LAG-3 and PD-L1, that is within a 5 fold range of the potency in blocking ligand or receptor binding to LAG-3 and/or PD-L1, preferably LAG-3 and PD-L1, of the combination of reference antibodies, and includes a 5, 4, 3, or 2 fold, preferably a 3 or 2 fold, deviation from the potency in blocking ligand or receptor binding to LAG-3 and/or PD-L1, preferably LAG-3 and PD-L1, of the combination of reference antibodies. In certain embodiments, the combination of reference antibodies comprises an analog of anti-LAG-3 antibody relatlimab and an analog of anti-PD-L1 antibody atezolizumab. A relatlimab analog has the same heavy chain variable region sequence (SEQ ID NO: 27) as relatlimab. A relatlimab analog antibody has the same light chain variable region sequence (SEQ ID NO: 28) as relatlimab. An atezolizumab analog has the same heavy chain variable region sequence (SEQ ID NO: 25) as atezolizumab. An atezolizumab analog antibody has the same light chain variable region sequence (SEQ ID NO: 26) as atezolizumab. The reference antibodies are preferably produced using the same production method as the multispecific antibody or variant thereof. Preferably, the LAG-3 and PD-L1 binding domains of the multispecific antibody or variant thereof of the present disclosure and the combination of reference antibodies are used at equimolar concentrations, i.e. when the multispecific antibody or variant thereof is used at 100 μg/mL, the combination of reference antibodies comprises 50 μg/mL of the LAG-3 binding reference antibody and 50 μg/mL of the PD-L1 binding reference antibody.

In certain embodiments, the present disclosure provides a multispecific antibody, or variant thereof that maintains the binding specificity of the antibody, wherein the antibody or variant thereof comprises a binding domain that specifically binds to an extracellular domain of LAG-3 and a binding domain that specifically binds to an extracellular domain of a protein of the B7 family.

In certain embodiments, the LAG-3 binding domain of a multispecific antibody or variant thereof of the present disclosure comprises a heavy chain variable region, wherein the heavy chain variable region comprises the heavy chain CDR1 (HCDR1), heavy chain CDR2 (HCDR2), and heavy chain CDR3 (HCDR3) of one of the heavy chain variable regions having an amino acid sequence as set forth in SEQ ID NO: 1-17 or SEQ ID NO: 52-59. The HCDRs, according to Kabat, are indicated in bold and underlined in the list of sequences provided herein. CDR sequences can be defined using different methods, including, but not limited to, according to the Kabat numbering scheme (Kabat et al., J. Biol. Chem. 252:6609-6616 (1977); and/or Kabat et al., U.S. Dept. of Health and Human Services, “Sequences of proteins of immunological interest” (1991)), the Chothia numbering scheme (Chothia et al., J. Mol. Biol. 196:901-917 (1987); Chothia et al., Nature 342: 877-883, 1989; and/or Al-Lazikani B. et al., J. Mol. Biol., 273: 927-948 (1997)), the numbering system of Honegger and Plukthun (Honegger and Plückthun, J. Mol. Biol., 309:657-670 (2001)), the numbering system of MacCallum (MacCallum et al., J. Mol. Biol. 262:732-745 (1996); and/or Abhinandan and Martin, Mol. Immunol., 45: 3832-3839 (2008)), the numbering system of Lefranc (Lefranc M. P. et al., Dev. Comp. Immunol., 27: 55-77 (2003); and/or Honegger and Plückthun, J. Mol. Biol., 309:657-670 (2001)), or according to IMGT (discussed in Giudicelli et al., Nucleic Acids Res. 25: 206-211 (1997)).

Each of these numbering schemes base their definition of CDRs on a predicted contribution of amino acid residues in the heavy or light chain variable region to antigen binding. Hence, each method to identify CDRs can be used to identify the CDRs of the binding domains of the present disclosure. In certain embodiments, the heavy chain CDRs of a binding domain of the present disclosure is according to Kabat, Chothia, or IMGT. In certain embodiments, the heavy chain CDRs of a binding domain of the present disclosure is according to Kabat. In certain embodiments, the heavy chain CDRs of a binding domain of the present disclosure is according to Chothia. In certain embodiments, the heavy chain CDRs of a binding domain of the present disclosure is according to IMGT.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 1;
heavy chain CDR1 (HCDR1), heavy chain CDR2 (HCDR2), and heavy chain CDR3 (HCDR3), of a heavy chain variable region having an amino acid sequence as set forth in SEQ ID NO: 2;
heavy chain CDR1 (HCDR1), heavy chain CDR2 (HCDR2), and heavy chain CDR3 (HCDR3), of a heavy chain variable region having an amino acid sequence as set forth in SEQ ID NO: 3;
heavy chain CDR1 (HCDR1), heavy chain CDR2 (HCDR2), and heavy chain CDR3 (HCDR3), of a heavy chain variable region having an amino acid sequence as set forth in SEQ ID NO: 4;
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 6;
heavy chain CDR1 (HCDR1), heavy chain CDR2 (HCDR2), and heavy chain CDR3 (HCDR3), of a heavy chain variable region having an amino acid sequence as set forth in SEQ ID NO: 7;
heavy chain CDR1 (HCDR1), heavy chain CDR2 (HCDR2), and heavy chain CDR3 (HCDR3), of a heavy chain variable region having an amino acid sequence as set forth in SEQ ID NO: 8;
heavy chain CDR1 (HCDR1), heavy chain CDR2 (HCDR2), and heavy chain CDR3 (HCDR3), of a heavy chain variable region having an amino acid sequence as set forth in SEQ ID NO: 9;
heavy chain CDR1 (HCDR1), heavy chain CDR2 (HCDR2), and heavy chain CDR3 (HCDR3), of a heavy chain variable region having an amino acid sequence as set forth in SEQ ID NO: 10;
heavy chain CDR1 (HCDR1), heavy chain CDR2 (HCDR2), and heavy chain CDR3 (HCDR3), of a heavy chain variable region having an amino acid sequence as set forth in SEQ ID NO: 11;
heavy chain CDR1 (HCDR1), heavy chain CDR2 (HCDR2), and heavy chain CDR3 (HCDR3), of a heavy chain variable region having an amino acid sequence as set forth in SEQ ID NO: 12;
heavy chain CDR1 (HCDR1), heavy chain CDR2 (HCDR2), and heavy chain CDR3 (HCDR3), of a heavy chain variable region having an amino acid sequence as set forth in SEQ ID NO: 13;
heavy chain CDR1 (HCDR1), heavy chain CDR2 (HCDR2), and heavy chain CDR3 (HCDR3), of a heavy chain variable region having an amino acid sequence as set forth in SEQ ID NO: 14;
heavy chain CDR1 (HCDR1), heavy chain CDR2 (HCDR2), and heavy chain CDR3 (HCDR3), of a heavy chain variable region having an amino acid sequence as set forth in SEQ ID NO: 15;
heavy chain CDR1 (HCDR1), heavy chain CDR2 (HCDR2), and heavy chain CDR3 (HCDR3), of a heavy chain variable region having an amino acid sequence as set forth in SEQ ID NO: 16;
heavy chain CDR1 (HCDR1), heavy chain CDR2 (HCDR2), and heavy chain CDR3 (HCDR3), of a heavy chain variable region having an amino acid sequence as set forth in SEQ ID NO: 17,
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59;
wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 60, SEQ ID NO: 61 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 60, SEQ ID NO: 63 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 67, SEQ ID NO: 68 and SEQ ID NO: 69, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 73, SEQ ID NO: 74 and SEQ ID NO: 75, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 77 and SEQ ID NO: 78, respectively;
heavy chain CDR1 (HCDR1), heavy chain CDR2 (HCDR2), and heavy chain CDR3 (HCDR3), having an amino acid sequence as set forth in SEQ ID NO: 79, SEQ ID NO: 80 and SEQ ID NO: 81, respectively;
heavy chain CDR1 (HCDR1), heavy chain CDR2 (HCDR2), and heavy chain CDR3 (HCDR3), having an amino acid sequence as set forth in SEQ ID NO: 82, SEQ ID NO: 83 and SEQ ID NO: 84, respectively;
heavy chain CDR1 (HCDR1), heavy chain CDR2 (HCDR2), and heavy chain CDR3 (HCDR3), having an amino acid sequence as set forth in SEQ ID NO: 85, SEQ ID NO: 86 and SEQ ID NO: 87, respectively;
heavy chain CDR1 (HCDR1), heavy chain CDR2 (HCDR2), and heavy chain CDR3 (HCDR3), an amino acid sequence as set forth in SEQ ID NO: 88, SEQ ID NO: 89 and SEQ ID NO: 90, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 91 and SEQ ID NO: 92, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 91 and SEQ ID NO: 93, respectively;
heavy chain CDR1 (HCDR1), heavy chain CDR2 (HCDR2), and heavy chain CDR3 (HCDR3), having an amino acid sequence as set forth in SEQ ID NO: 94, SEQ ID NO: 95 and SEQ ID NO: 96, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 99, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 100, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively,
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively;
wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 1;
heavy chain CDR1 (HCDR1), heavy chain CDR2 (HCDR2), and heavy chain CDR3 (HCDR3), of a heavy chain variable region having an amino acid sequence as set forth in SEQ ID NO: 3;
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 6;
heavy chain CDR1 (HCDR1), heavy chain CDR2 (HCDR2), and heavy chain CDR3 (HCDR3), of a heavy chain variable region having an amino acid sequence as set forth in SEQ ID NO: 15;
heavy chain CDR1 (HCDR1), heavy chain CDR2 (HCDR2), and heavy chain CDR3 (HCDR3), of a heavy chain variable region having an amino acid sequence as set forth in SEQ ID NO: 16;
heavy chain CDR1 (HCDR1), heavy chain CDR2 (HCDR2), and heavy chain CDR3 (HCDR3), of a heavy chain variable region having an amino acid sequence as set forth in SEQ ID NO: 17;
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59; wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 60, SEQ ID NO: 61 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 73, SEQ ID NO: 74 and SEQ ID NO: 75, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 99, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 100, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively;
wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 17;
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59;
wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively;
wherein each of the HCDRs may comprise at most three, two, or one amino acid substitution.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5,
wherein each of the HCDRs may comprise at most three, two, or one amino acid substitution.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), of a heavy chain variable region having an amino acid sequence as set forth in SEQ ID NO: 52,
wherein each of the HCDRs may comprise at most three, two, or one amino acid substitution.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
wherein each of the HCDRs may comprise at most three, two, or one amino acid substitution.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
wherein each of the HCDRs may comprise at most three, two, or one amino acid substitution.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 1;
heavy chain CDR1 (HCDR1), heavy chain CDR2 (HCDR2), and heavy chain CDR3 (HCDR3), of a heavy chain variable region having an amino acid sequence as set forth in SEQ ID NO: 2;
heavy chain CDR1 (HCDR1), heavy chain CDR2 (HCDR2), and heavy chain CDR3 (HCDR3), of a heavy chain variable region having an amino acid sequence as set forth in SEQ ID NO: 3;
heavy chain CDR1 (HCDR1), heavy chain CDR2 (HCDR2), and heavy chain CDR3 (HCDR3), of a heavy chain variable region having an amino acid sequence as set forth in SEQ ID NO: 4;
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 6;
heavy chain CDR1 (HCDR1), heavy chain CDR2 (HCDR2), and heavy chain CDR3 (HCDR3), of a heavy chain variable region having an amino acid sequence as set forth in SEQ ID NO: 7;
heavy chain CDR1 (HCDR1), heavy chain CDR2 (HCDR2), and heavy chain CDR3 (HCDR3), of a heavy chain variable region having an amino acid sequence as set forth in SEQ ID NO: 8;
heavy chain CDR1 (HCDR1), heavy chain CDR2 (HCDR2), and heavy chain CDR3 (HCDR3), of a heavy chain variable region having an amino acid sequence as set forth in SEQ ID NO: 9;
heavy chain CDR1 (HCDR1), heavy chain CDR2 (HCDR2), and heavy chain CDR3 (HCDR3), of a heavy chain variable region having an amino acid sequence as set forth in SEQ ID NO: 10;
heavy chain CDR1 (HCDR1), heavy chain CDR2 (HCDR2), and heavy chain CDR3 (HCDR3), of a heavy chain variable region having an amino acid sequence as set forth in SEQ ID NO: 11;
heavy chain CDR1 (HCDR1), heavy chain CDR2 (HCDR2), and heavy chain CDR3 (HCDR3), of a heavy chain variable region having an amino acid sequence as set forth in SEQ ID NO: 12;
heavy chain CDR1 (HCDR1), heavy chain CDR2 (HCDR2), and heavy chain CDR3 (HCDR3), of a heavy chain variable region having an amino acid sequence as set forth in SEQ ID NO: 13;
heavy chain CDR1 (HCDR1), heavy chain CDR2 (HCDR2), and heavy chain CDR3 (HCDR3), of a heavy chain variable region having an amino acid sequence as set forth in SEQ ID NO: 14;
heavy chain CDR1 (HCDR1), heavy chain CDR2 (HCDR2), and heavy chain CDR3 (HCDR3), of a heavy chain variable region having an amino acid sequence as set forth in SEQ ID NO: 15;
heavy chain CDR1 (HCDR1), heavy chain CDR2 (HCDR2), and heavy chain CDR3 (HCDR3), of a heavy chain variable region having an amino acid sequence as set forth in SEQ ID NO: 16;
heavy chain CDR1 (HCDR1), heavy chain CDR2 (HCDR2), and heavy chain CDR3 (HCDR3), of a heavy chain variable region having an amino acid sequence as set forth in SEQ ID NO: 17;
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 60, SEQ ID NO: 61 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 60, SEQ ID NO: 63 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 67, SEQ ID NO: 68 and SEQ ID NO: 69, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 73, SEQ ID NO: 74 and SEQ ID NO: 75, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 77 and SEQ ID NO: 78, respectively;
heavy chain CDR1 (HCDR1), heavy chain CDR2 (HCDR2), and heavy chain CDR3 (HCDR3), having an amino acid sequence as set forth in SEQ ID NO: 79, SEQ ID NO: 80 and SEQ ID NO: 81, respectively;
heavy chain CDR1 (HCDR1), heavy chain CDR2 (HCDR2), and heavy chain CDR3 (HCDR3), having an amino acid sequence as set forth in SEQ ID NO: 82, SEQ ID NO: 83 and SEQ ID NO: 84, respectively;
heavy chain CDR1 (HCDR1), heavy chain CDR2 (HCDR2), and heavy chain CDR3 (HCDR3), having an amino acid sequence as set forth in SEQ ID NO: 85, SEQ ID NO: 86 and SEQ ID NO: 87, respectively;
heavy chain CDR1 (HCDR1), heavy chain CDR2 (HCDR2), and heavy chain CDR3 (HCDR3), having an amino acid sequence as set forth in SEQ ID NO: 88, SEQ ID NO: 89 and SEQ ID NO: 90, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 91 and SEQ ID NO: 92, respectively;
heavy chain CDR1 (HCDR1), heavy chain CDR2 (HCDR2), and heavy chain CDR3 (HCDR3), having an amino acid sequence as set forth in SEQ ID NO: 76, SEQ ID NO: 91 and SEQ ID NO: 93, respectively;
heavy chain CDR1 (HCDR1), heavy chain CDR2 (HCDR2), and heavy chain CDR3 (HCDR3), having an amino acid sequence as set forth in SEQ ID NO: 94, SEQ ID NO: 95 and SEQ ID NO: 96, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 99, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 100, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 1;
heavy chain CDR1 (HCDR1), heavy chain CDR2 (HCDR2), and heavy chain CDR3 (HCDR3), of a heavy chain variable region having an amino acid sequence as set forth in SEQ ID NO: 3;
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 6;
heavy chain CDR1 (HCDR1), heavy chain CDR2 (HCDR2), and heavy chain CDR3 (HCDR3), of a heavy chain variable region having an amino acid sequence as set forth in SEQ ID NO: 15;
heavy chain CDR1 (HCDR1), heavy chain CDR2 (HCDR2), and heavy chain CDR3 (HCDR3), of a heavy chain variable region having an amino acid sequence as set forth in SEQ ID NO: 16;
heavy chain CDR1 (HCDR1), heavy chain CDR2 (HCDR2), and heavy chain CDR3 (HCDR3), of a heavy chain variable region having an amino acid sequence as set forth in SEQ ID NO: 17;
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 60, SEQ ID NO: 61 and SEQ ID NO: 62, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 73, SEQ ID NO: 74 and SEQ ID NO: 75, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 99, respectively;
heavy chain CDR1 (HCDR1), heavy chain CDR2 (HCDR2), and heavy chain CDR3 (HCDR3), having an amino acid sequence as set forth in SEQ ID NO: 97, SEQ ID NO: 98 and SEQ ID NO: 100, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 5;
heavy chain CDR1 (HCDR1), heavy chain CDR2 (HCDR2), and heavy chain CDR3 (HCDR3), of a heavy chain variable region having an amino acid sequence as set forth in SEQ ID NO: 17;
heavy chain CDR1 (HCDR1), heavy chain CDR2 (HCDR2), and heavy chain CDR3 (HCDR3), of a heavy chain variable region having an amino acid sequence as set forth in SEQ ID NO: 52;
heavy chain CDR1 (HCDR1), heavy chain CDR2 (HCDR2), and heavy chain CDR3 (HCDR3), of a heavy chain variable region having an amino acid sequence as set forth in SEQ ID NO: 53;
heavy chain CDR1 (HCDR1), heavy chain CDR2 (HCDR2), and heavy chain CDR3 (HCDR3), of a heavy chain variable region having an amino acid sequence as set forth in SEQ ID NO: 54;
heavy chain CDR1 (HCDR1), heavy chain CDR2 (HCDR2), and heavy chain CDR3 (HCDR3), of a heavy chain variable region having an amino acid sequence as set forth in SEQ ID NO: 55;
heavy chain CDR1 (HCDR1), heavy chain CDR2 (HCDR2), and heavy chain CDR3 (HCDR3), of a heavy chain variable region having an amino acid sequence as set forth in SEQ ID NO: 56;
heavy chain CDR1 (HCDR1), heavy chain CDR2 (HCDR2), and heavy chain CDR3 (HCDR3), of a heavy chain variable region having an amino acid sequence as set forth in SEQ ID NO: 57;
heavy chain CDR1 (HCDR1), heavy chain CDR2 (HCDR2), and heavy chain CDR3 (HCDR3), of a heavy chain variable region having an amino acid sequence as set forth in SEQ ID NO: 58; or
heavy chain CDR1 (HCDR1), heavy chain CDR2 (HCDR2), and heavy chain CDR3 (HCDR3), of a heavy chain variable region having an amino acid sequence as set forth in SEQ ID NO: 59.

In certain embodiments, the LAG-3 binding domain of the multispecific antibody or variant thereof comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively;
heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively;
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; or
heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), of a heavy chain variable region having an amino acid sequence as set forth in SEQ ID NO: 52.

In certain embodiments, the LAG-3 binding domain of the multispecific antibody or variant thereof comprises a heavy chain variable region comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively.

In certain embodiments, the LAG-3 binding domain of the multispecific antibody or variant thereof comprises a heavy chain variable region having an amino acid sequence as set forth in any one of SEQ ID NO: 1-17 or SEQ ID NO: 52-59, or having at least 80%, preferably 85%, more preferably 90%, or most preferably 95% sequence identity thereto.

In certain embodiments, a LAG-3 binding domain of the multispecific antibody or variant thereof includes LAG-3 binding domain variants, wherein each of the HCDRs may comprise at most three, two, or one amino acid substitution. Such variants are expected to retain LAG-3 binding specificity.

For example, suitable positions for introducing an amino acid variation include, but are not limited to, the second, and/or third amino acid of HCDR1; the third, seventh, tenth, thirteenth, and/or sixteenth amino acid of HCDR2; and/or the first amino acid of HCDR3. CDR sequences according to Kabat, are indicated in bold and underlined in the list of sequences provided herein.

In certain embodiments, the anti-human LAG-3 binding domain comprises:
HCDR1 having amino acid sequence SX₁X₂WS, wherein
   X₁ can be Y or F;
   X₂ can be Y or S; and/or
HCDR2 having amino acid sequence YIX₁YSGX₂TNX₃NPX₄LKX₅, wherein
   X₁ can be Y or D;
   X₂ can be S, or T;
   X₃ can be Y or F;
   X₄ can be S, or F;
   X₅ can be S or I; and/or
HCDR3 having amino acid sequence X₁LLYKWNYVEGFDI, wherein
   X₁ can be D or H.

For example, suitable positions for introducing an amino acid variation include, but are not limited to, the first, third and/or fourth amino acid of HCDR1; the seventh, tenth, and/or twelfth amino acid of HCDR2; and/or the third amino acid of HCDR3. CDR sequences according to Kabat are indicated in bold and underlined in the list of sequences provided herein.

In certain embodiments, the anti-human LAG-3 binding domain comprises:
HCDR1 having amino acid sequence X₁YX₂X₃H, wherein
   X₁ can be S, N, or R;
   X₂ can be G or D;
   X₃ can be M, T or I; and/or
HCDR2 having amino acid sequence VISYDGX₁NKX₂YX₃DSVKG, wherein
   X₁ can be S or N;
   X₂ can be Y, F, or H;
   X₃ can be A, E, or V; and/or
HCDR3 having amino acid sequence ERX₁WDVFDI, wherein
   X₁ can be G or D.

For example, suitable positions for introducing an amino acid variation include, but are not limited to, the first, and/or third amino acid of HCDR1; the fifth, and/or eighth amino acid of HCDR2; and/or the third amino acid of HCDR3. CDR sequences according to Kabat, are indicated in bold and underlined in the list of sequences provided herein.

In certain embodiments, the anti-human LAG-3 binding domain comprises:
HCDR1 having amino acid sequence X₁YX₂MH, wherein
   X₁ can be S or N;
   X₂ can be G or A; and/or
HCDR2 having amino acid sequence VISYX₁GSX₂KYYADSVKG, wherein
   X₁ can be D or H;
   X₂ can be N or D; and/or
HCDR3 having amino acid sequence DGDNWDX₁FDI, wherein
   X₁ can be V or A.

In certain embodiments, a LAG-3 binding domain of the multispecific antibody or variant thereof also includes LAG-3 binding domain variants, which, in addition to variations in the HCDRs, comprise one or more variations in the framework regions. In certain embodiments, a LAG-3 binding domain variant of the multispecific antibody or variant thereof comprises no variations in the CDR regions but comprises one or more variations in the framework regions. Such variants have at least 80%, preferably 85%, more preferably 90%, or most preferably 95% sequence identity to the sequences disclosed herein, and are expected to retain LAG-3 binding specificity. Thus, in certain embodiments, a LAG-3 binding domain of the present disclosure comprises:
a heavy chain variable region having at least 80%, preferably 85%, more preferably 90%, or most preferably 95% sequence identity to the amino acid sequence as set forth in SEQ ID NO: 1, which heavy chain variable region comprises the HCDR1, HCDR2, and HCDR3 of the amino acid sequence as set forth in SEQ ID NO: 1;
a heavy chain variable region having at least 80%, preferably 85%, more preferably 90%, or most preferably 95% sequence identity to the amino acid sequence as set forth in SEQ ID NO: 2, which heavy chain variable region comprises the HCDR1, HCDR2, and HCDR3 of the amino acid sequence as set forth in SEQ ID NO: 2;
a heavy chain variable region having at least 80%, preferably 85%, more preferably 90%, or most preferably 95% sequence identity to the amino acid sequence as set forth in SEQ ID NO: 3, which heavy chain variable region comprises the HCDR1, HCDR2, and HCDR3 of the amino acid sequence as set forth in SEQ ID NO: 3;
a heavy chain variable region having at least 80%, preferably 85%, more preferably 90%, or most preferably 95% sequence identity to the amino acid sequence as set forth in SEQ ID NO: 4, which heavy chain variable region comprises the HCDR1, HCDR2, and HCDR3 of the amino acid sequence as set forth in SEQ ID NO: 4;
a heavy chain variable region having at least 80%, preferably 85%, more preferably 90%, or most preferably 95% sequence identity to the amino acid sequence as set forth in SEQ ID NO: 5, which heavy chain variable region comprises the HCDR1, HCDR2, and HCDR3 of the amino acid sequence as set forth in SEQ ID NO: 5;
a heavy chain variable region having at least 80%, preferably 85%, more preferably 90%, or most preferably 95% sequence identity to the amino acid sequence as set forth in SEQ ID NO: 6, which heavy chain variable region comprises the HCDR1, HCDR2, and HCDR3 of the amino acid sequence as set forth in SEQ ID NO: 6;
a heavy chain variable region having at least 80%, preferably 85%, more preferably 90%, or most preferably 95% sequence identity to the amino acid sequence as set forth in SEQ ID NO: 7, which heavy chain variable region comprises the HCDR1, HCDR2, and HCDR3 of the amino acid sequence as set forth in SEQ ID NO: 7;
a heavy chain variable region having at least 80%, preferably 85%, more preferably 90%, or most preferably 95% sequence identity to the amino acid sequence as set forth in SEQ ID NO: 8, which heavy chain variable region comprises the HCDR1, HCDR2, and HCDR3 of the amino acid sequence as set forth in SEQ ID NO: 8;
a heavy chain variable region having at least 80%, preferably 85%, more preferably 90%, or most preferably 95% sequence identity to the amino acid sequence as set forth in SEQ ID NO: 9, which heavy chain variable region comprises the HCDR1, HCDR2, and HCDR3 of the amino acid sequence as set forth in SEQ ID NO: 9;
a heavy chain variable region having at least 80%, preferably 85%, more preferably 90%, or most preferably 95% sequence identity to the amino acid sequence as set forth in SEQ ID NO: 10, which heavy chain variable region comprises the HCDR1, HCDR2, and HCDR3 of the amino acid sequence as set forth in SEQ ID NO: 10;
a heavy chain variable region having at least 80%, preferably 85%, more preferably 90%, or most preferably 95% sequence identity to the amino acid sequence as set forth in SEQ ID NO: 11, which heavy chain variable region comprises the HCDR1, HCDR2, and HCDR3 of the amino acid sequence as set forth in SEQ ID NO: 11;
a heavy chain variable region having at least 80%, preferably 85%, more preferably 90%, or most preferably 95% sequence identity to the amino acid sequence as set forth in SEQ ID NO: 12, which heavy chain variable region comprises the HCDR1, HCDR2, and HCDR3 of the amino acid sequence as set forth in SEQ ID NO: 12;
a heavy chain variable region having at least 80%, preferably 85%, more preferably 90%, or most preferably 95% sequence identity to the amino acid sequence as set forth in SEQ ID NO: 13, which heavy chain variable region comprises the HCDR1, HCDR2, and HCDR3 of the amino acid sequence as set forth in SEQ ID NO: 13;
a heavy chain variable region having at least 80%, preferably 85%, more preferably 90%, or most preferably 95% sequence identity to the amino acid sequence as set forth in SEQ ID NO: 14, which heavy chain variable region comprises the HCDR1, HCDR2, and HCDR3 of the amino acid sequence as set forth in SEQ ID NO: 14;
a heavy chain variable region having at least 80%, preferably 85%, more preferably 90%, or most preferably 95% sequence identity to the amino acid sequence as set forth in SEQ ID NO: 15, which heavy chain variable region comprises the HCDR1, HCDR2, and HCDR3 of the amino acid sequence as set forth in SEQ ID NO: 15;
a heavy chain variable region having at least 80%, preferably 85%, more preferably 90%, or most preferably 95% sequence identity to the amino acid sequence as set forth in SEQ ID NO: 16, which heavy chain variable region comprises the HCDR1, HCDR2, and HCDR3 of the amino acid sequence as set forth in SEQ ID NO: 16;
a heavy chain variable region having at least 80%, preferably 85%, more preferably 90%, or most preferably 95% sequence identity to the amino acid sequence as set forth in SEQ ID NO: 17, which heavy chain variable region comprises the HCDR1, HCDR2, and HCDR3 of the amino acid sequence as set forth in SEQ ID NO: 17;
a heavy chain variable region having at least 80%, preferably 85%, more preferably 90%, or most preferably 95% sequence identity to the amino acid sequence as set forth in SEQ ID NO: 52, which heavy chain variable region comprises the HCDR1, HCDR2, and HCDR3 of the amino acid sequence as set forth in SEQ ID NO: 52;
a heavy chain variable region having at least 80%, preferably 85%, more preferably 90%, or most preferably 95% sequence identity to the amino acid sequence as set forth in SEQ ID NO: 53, which heavy chain variable region comprises the HCDR1, HCDR2, and HCDR3 of the amino acid sequence as set forth in SEQ ID NO: 53;
a heavy chain variable region having at least 80%, preferably 85%, more preferably 90%, or most preferably 95% sequence identity to the amino acid sequence as set forth in SEQ ID NO: 54, which heavy chain variable region comprises the HCDR1, HCDR2, and HCDR3 of the amino acid sequence as set forth in SEQ ID NO: 54;
a heavy chain variable region having at least 80%, preferably 85%, more preferably 90%, or most preferably 95% sequence identity to the amino acid sequence as set forth in SEQ ID NO: 55, which heavy chain variable region comprises the HCDR1, HCDR2, and HCDR3 of the amino acid sequence as set forth in SEQ ID NO: 55;
a heavy chain variable region having at least 80%, preferably 85%, more preferably 90%, or most preferably 95% sequence identity to the amino acid sequence as set forth in SEQ ID NO: 56, which heavy chain variable region comprises the HCDR1, HCDR2, and HCDR3 of the amino acid sequence as set forth in SEQ ID NO: 56;
a heavy chain variable region having at least 80%, preferably 85%, more preferably 90%, or most preferably 95% sequence identity to the amino acid sequence as set forth in SEQ ID NO: 57, which heavy chain variable region comprises the HCDR1, HCDR2, and HCDR3 of the amino acid sequence as set forth in SEQ ID NO: 57;
a heavy chain variable region having at least 80%, preferably 85%, more preferably 90%, or most preferably 95% sequence identity to the amino acid sequence as set forth in SEQ ID NO: 58, which heavy chain variable region comprises the HCDR1, HCDR2, and HCDR3 of the amino acid sequence as set forth in SEQ ID NO: 58; or
a heavy chain variable region having at least 80%, preferably 85%, more preferably 90%, or most preferably 95% sequence identity to the amino acid sequence as set forth in SEQ ID NO: 59, which heavy chain variable region comprises the HCDR1, HCDR2, and HCDR3 of the amino acid sequence as set forth in SEQ ID NO: 59.

In certain embodiments, a LAG-3 binding domain of the present disclosure comprises:
a heavy chain variable region having at least 80%, preferably 85%, more preferably 90%, or most preferably 95% sequence identity to the amino acid sequence as set forth in SEQ ID NO: 1, which heavy chain variable region comprises the HCDR1, HCDR2, and HCDR3 amino acid sequence as set forth in SEQ ID NO: 60, SEQ ID NO: 61 and SEQ ID NO: 62, respectively;
a heavy chain variable region having at least 80%, preferably 85%, more preferably 90%, or most preferably 95% sequence identity to the amino acid sequence as set forth in SEQ ID NO: 2, which heavy chain variable region comprises the HCDR1, HCDR2, and HCDR3 amino acid sequence as set forth in SEQ ID NO: 60, SEQ ID NO: 63 and SEQ ID NO: 62, respectively;
a heavy chain variable region having at least 80%, preferably 85%, more preferably 90%, or most preferably 95% sequence identity to the amino acid sequence as set forth in SEQ ID NO: 3, which heavy chain variable region comprises the HCDR1, HCDR2, and HCDR3 amino acid sequence as set forth in SEQ ID NO: 64, SEQ ID NO: 65 and SEQ ID NO: 66, respectively;
a heavy chain variable region having at least 80%, preferably 85%, more preferably 90%, or most preferably 95% sequence identity to the amino acid sequence as set forth in SEQ ID NO: 4, which heavy chain variable region comprises the HCDR1, HCDR2, and HCDR3 amino acid sequence as set forth in SEQ ID NO: 67, SEQ ID NO: 68 and SEQ ID NO: 69, respectively;
a heavy chain variable region having at least 80%, preferably 85%, more preferably 90%, or most preferably 95% sequence identity to the amino acid sequence as set forth in SEQ ID NO: 5, which heavy chain variable region comprises the HCDR1, HCDR2, and HCDR3 amino acid sequence as set forth in SEQ ID NO: 70, SEQ ID NO: 71 and SEQ ID NO: 72, respectively;
a heavy chain variable region having at least 80%, preferably 85%, more preferably 90%, or most preferably 95% sequence identity to the amino acid sequence as set forth in SEQ ID NO: 6, which heavy chain variable region comprises the HCDR1, HCDR2, and HCDR3 amino acid sequence as set forth in SEQ ID NO: 73, SEQ ID NO: 74 and SEQ ID NO: 75, respectively;
a heavy chain variable region having at least 80%, preferably 85%, more preferably 90%, or most preferably 95% sequence identity to the amino acid sequence as set forth in SEQ ID NO: 7, which heavy chain variable region comprises the HCDR1, HCDR2, and HCDR3 amino acid sequence as set forth in SEQ ID NO: 76, SEQ ID NO: 77 and SEQ ID NO: 78, respectively;
a heavy chain variable region having at least 80%, preferably 85%, more preferably 90%, or most preferably 95% sequence identity to the amino acid sequence as set forth in SEQ ID NO: 8, which heavy chain variable region comprises the HCDR1, HCDR2, and HCDR3 amino acid sequence as set forth in SEQ ID NO: 79, SEQ ID NO: 80 and SEQ ID NO: 81, respectively;
a heavy chain variable region having at least 80%, preferably 85%, more preferably 90%, or most preferably 95% sequence identity to the amino acid sequence as set forth in SEQ ID NO: 9, which heavy chain variable region comprises the HCDR1, HCDR2, and HCDR3 amino acid sequence as set forth in SEQ ID NO: 82, SEQ ID NO: 83 and SEQ ID NO: 84, respectively;
a heavy chain variable region having at least 80%, preferably 85%, more preferably 90%, or most preferably 95% sequence identity to the amino acid sequence as set forth in SEQ ID NO: 10, which heavy chain variable region comprises the HCDR1, HCDR2, and HCDR3 amino acid sequence as set forth in SEQ ID NO: 85, SEQ ID NO: 86 and SEQ ID NO: 87, respectively;
a heavy chain variable region having at least 80%, preferably 85%, more preferably 90%, or most preferably 95% sequence identity to the amino acid sequence as set forth in SEQ ID NO: 11, which heavy chain variable region comprises the HCDR1, HCDR2, and HCDR3 amino acid sequence as set forth in SEQ ID NO: 88, SEQ ID NO: 89 and SEQ ID NO: 90, respectively;
a heavy chain variable region having at least 80%, preferably 85%, more preferably 90%, or most preferably 95% sequence identity to the amino acid sequence as set forth in SEQ ID NO: 12, which heavy chain variable region comprises the HCDR1, HCDR2, and HCDR3 amino acid sequence as set forth in SEQ ID NO: 76, SEQ ID NO: 91 and SEQ ID NO: 92, respectively;
a heavy chain variable region having at least 80%, preferably 85%, more preferably 90%, or most preferably 95% sequence identity to the amino acid sequence as set forth in SEQ ID NO: 13, which heavy chain variable region comprises the HCDR1, HCDR2, and HCDR3 amino acid sequence as set forth in SEQ ID NO: 76, SEQ ID NO: 91 and SEQ ID NO: 93, respectively;
a heavy chain variable region having at least 80%, preferably 85%, more preferably 90%, or most preferably 95% sequence identity to the amino acid sequence as set forth in SEQ ID NO: 14, which heavy chain variable region comprises the HCDR1, HCDR2, and HCDR3 amino acid sequence as set forth in SEQ ID NO: 94, SEQ ID NO: 95 and SEQ ID NO: 96, respectively;
a heavy chain variable region having at least 80%, preferably 85%, more preferably 90%, or most preferably 95% sequence identity to the amino acid sequence as set forth in SEQ ID NO: 15, which heavy chain variable region comprises the HCDR1, HCDR2, and HCDR3 amino acid sequence as set forth in SEQ ID NO: 97, SEQ ID NO: 98 and SEQ ID NO: 99, respectively;
a heavy chain variable region having at least 80%, preferably 85%, more preferably 90%, or most preferably 95% sequence identity to the amino acid sequence as set forth in SEQ ID NO: 16, which heavy chain variable region comprises the HCDR1, HCDR2, and HCDR3 amino acid sequence as set forth in SEQ ID NO: 97, SEQ ID NO: 98 and SEQ ID NO: 100, respectively;
a heavy chain variable region having at least 80%, preferably 85%, more preferably 90%, or most preferably 95% sequence identity to the amino acid sequence as set forth in SEQ ID NO: 17, which heavy chain variable region comprises the HCDR1, HCDR2, and HCDR3 amino acid sequence as set forth in SEQ ID NO: 64, SEQ ID NO: 65 and SEQ ID NO: 66, respectively;
a heavy chain variable region having at least 80%, preferably 85%, more preferably 90%, or most preferably 95% sequence identity to the amino acid sequence as set forth in SEQ ID NO: 52, which heavy chain variable region comprises the HCDR1, HCDR2, and HCDR3 amino acid sequence as set forth in SEQ ID NO: 70, SEQ ID NO: 71 and SEQ ID NO: 118, respectively;
a heavy chain variable region having at least 80%, preferably 85%, more preferably 90%, or most preferably 95% sequence identity to the amino acid sequence as set forth in SEQ ID NO: 53, which heavy chain variable region comprises the HCDR1, HCDR2, and HCDR3 amino acid sequence as set forth in SEQ ID NO: 64, SEQ ID NO: 65 and SEQ ID NO: 119, respectively;
a heavy chain variable region having at least 80%, preferably 85%, more preferably 90%, or most preferably 95% sequence identity to the amino acid sequence as set forth in SEQ ID NO: 54, which heavy chain variable region comprises the HCDR1, HCDR2, and HCDR3 amino acid sequence as set forth in SEQ ID NO: 64, SEQ ID NO: 65 and SEQ ID NO: 120, respectively;
a heavy chain variable region having at least 80%, preferably 85%, more preferably 90%, or most preferably 95% sequence identity to the amino acid sequence as set forth in SEQ ID NO: 55, which heavy chain variable region comprises the HCDR1, HCDR2, and HCDR3 amino acid sequence as set forth in SEQ ID NO: 64, SEQ ID NO: 65 and SEQ ID NO: 121, respectively;
a heavy chain variable region having at least 80%, preferably 85%, more preferably 90%, or most preferably 95% sequence identity to the amino acid sequence as set forth in SEQ ID NO: 56, which heavy chain variable region comprises the HCDR1, HCDR2, and HCDR3 amino acid sequence as set forth in SEQ ID NO: 64, SEQ ID NO: 65 and SEQ ID NO: 122, respectively;
a heavy chain variable region having at least 80%, preferably 85%, more preferably 90%, or most preferably 95% sequence identity to the amino acid sequence as set forth in SEQ ID NO: 57, which heavy chain variable region comprises the HCDR1, HCDR2, and HCDR3 amino acid sequence as set forth in SEQ ID NO: 64, SEQ ID NO: 65 and SEQ ID NO: 123, respectively;
a heavy chain variable region having at least 80%, preferably 85%, more preferably 90%, or most preferably 95% sequence identity to the amino acid sequence as set forth in SEQ ID NO: 58, which heavy chain variable region comprises the HCDR1, HCDR2, and HCDR3 amino acid sequence as set forth in SEQ ID NO: 64, SEQ ID NO: 65 and SEQ ID NO: 124, respectively; or
a heavy chain variable region having at least 80%, preferably 85%, more preferably 90%, or most preferably 95% sequence identity to the amino acid sequence as set forth in SEQ ID NO: 59, which heavy chain variable region comprises the HCDR1, HCDR2, and HCDR3 amino acid sequence as set forth in SEQ ID NO: 64, SEQ ID NO: 65 and SEQ ID NO: 125, respectively.

In certain embodiments, a LAG-3 binding domain of the multispecific antibody or variant thereof further comprises a light chain variable region. In certain embodiments, a LAG-3 binding domain of the multispecific antibody or variant thereof comprises a common light chain variable region. An example of a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 31, SEQ ID NO: 32, and SEQ ID NO: 33, respectively, wherein each of the LCDRs may comprise at most three, two, or one amino acid substitution. In certain embodiments, a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 31, SEQ ID NO: 32, and SEQ ID NO: 33, respectively. In certain embodiments, such light chain variable region may comprise a light chain variable region having an amino acid sequence as set forth in SEQ ID NO: 30, or having at least 80%, preferably 85%, more preferably 90%, or most preferably 95% sequence identity thereto. A light chain or light chain variable region comprising these LCDRs and/or light chain variable region is the light chain referred to in the art as VK1-39/JK1. This is a common light chain.

In certain embodiments, a LAG-3 binding domain of the present disclosure comprises a light chain variable region having at least 80%, preferably 85%, more preferably 90%, or most preferably 95% sequence identity to the amino acid sequence as set forth in SEQ ID NO: 30, which light chain variable region comprises a LCDR1 amino acid sequence as set forth in SEQ ID NO: 31; a LCDR2 amino acid sequence as set forth in SEQ ID NO: 32; and a LCDR3 amino acid sequence as set forth in SEQ ID NO: 33.

The term ‘common light chain’ according to the invention refers to a light chain that is capable of pairing with multiple different heavy chains, i.e. heavy chains having different antigen or epitope binding specificities. A common light chain is particularly useful in the generation of, for instance, bispecific antibodies, where antibody production is more efficient when both binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 34. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 34, wherein each of the LCDRs may comprise at most three, two, or one amino acid substitution. In certain embodiments, the light chain comprises a light chain variable region having an amino acid sequence as set forth in SEQ ID NO: 34, or having at least 80%, preferably 85%, more preferably 90%, or most preferably 95% sequence identity thereto; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 35. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 35, wherein each of the LCDRs may comprise at most three, two, or one amino acid substitution. In certain embodiments, the light chain comprises a light chain variable region having an amino acid sequence as set forth in SEQ ID NO: 35, or having at least 80%, preferably 85%, more preferably 90%, or most preferably 95% sequence identity thereto; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 36. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 36, wherein each of the LCDRs may comprise at most three, two, or one amino acid substitution. In certain embodiments, the light chain comprises a light chain variable region having an amino acid sequence as set forth in SEQ ID NO: 36, or having at least 80%, preferably 85%, more preferably 90%, or most preferably 95% sequence identity thereto; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 37. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 37, wherein each of the LCDRs may comprise at most three, two, or one amino acid substitution. In certain embodiments, the light chain comprises a light chain variable region having an amino acid sequence as set forth in SEQ ID NO: 37, or having at least 80%, preferably 85%, more preferably 90%, or most preferably 95% sequence identity thereto.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. A preferred amino acid sequence for VK1-39 is given as SEQ ID NO: 38. This is the sequence of the V-region. The V-region can be combined with one of five J-regions. Two preferred joined sequences are indicated as VK1-39/JK1 and VK1-39/JK5; alternative names are IgVκ1-39_01/IGJκ1_01 or IgVκ1-39_01/IGJκ5_01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. A preferred amino acid sequence for VK3-15 is given as SEQ ID NO: 39. This is the sequence of the V-region. The V-region can be combined with one of five J-regions. A preferred joined sequence is indicated as VK3-15/JK1; alternative name is Vκ3-15_01/IGJκ1_01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. A preferred amino acid sequence for VK3-20 is as SEQ ID NO: 40. This is the sequence of the V-region. The V-region can be combined with one of five J-regions. A preferred joined sequence is indicated as VK3-20/JK1; alternative name is IgVκ3-20_01/IGJκ1_01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. A preferred amino acid sequence for VL3-21 is given as SEQ ID NO: 41. This is the sequence of the V-region. The V-region can be combined with one of five J-regions. A preferred joined sequence is indicated as VL3-21/JL3; alternative name is IgVλ3-21/IGJλ3 (nomenclature according to the IMGT database worldwide web at imgt.org). This name is exemplary and encompasses allelic variants of the gene segments.

Further, any light chain variable region of a LAG-3 antibody available in the art may be used, or any other light chain variable region that can readily be obtained, such as from, for instance, an antibody display library by showing antigen binding activity when paired with a LAG-3 binding domain of the invention.

In certain embodiments, the LAG-3 binding domain of the multispecific antibody or variant thereof may further comprise a CH1 and CL region. Any CH1 domain may be used, in particular a human CH1 domain. An example of a suitable CH1 domain is provided by the amino acid sequence provided as SEQ ID NO: 42. Any CL domain may be used, in particular a human CL. An example of a suitable CL domain is provided by the amino acid sequence provided as SEQ ID NO: 43.

In certain embodiments, the multispecific antibody or variant thereof comprises a binding domain that specifically binds to an extracellular domain of LAG-3 and a binding domain that specifically binds to an extracellular domain of a protein of the B7 family.

The B7 family comprises a number of structurally related, cell-surface proteins, which bind to receptors on lymphocytes that regulate immune responses. Activation of lymphocytes is initiated by engagement of cell-surface, antigen-specific T-cell receptors or B-cell receptors. Additional signals delivered simultaneously by B7 ligands further determine the immune response of these cells. These so-called ‘costimulatory’ or ‘coinhibitory’ signals are delivered by B7 family members through the CD28 family of receptors on lymphocytes. Binding of B7-family members with costimulatory receptors augments immune responses, and binding with coinhibitory receptors attenuates immune responses. Presently the following members are believed to be part of this family: B7.1 (CD80), B7.2 (CD86), inducible costimulator ligand (ICOS-L), programmed death-1 ligand (PD-L1), programmed death-2 ligand (PD-L2), B7-H3 (CD276), B7-H4, B7-H5, B7-H6 and B7-H7. B7 family members are expressed in lymphoid and non-lymphoid tissues. Effects of members on regulating immune responses are shown in the development of immunodeficiency and autoimmune diseases in mice with mutations in B7-family genes. Manipulation of the signals delivered by B7 ligands has shown potential in the treatment of autoimmunity, inflammatory diseases and cancer.

In certain embodiments, the protein of the B7 family is selected from the group consisting of PD-L1, PD-L2, CD80, CD86, B7-H4, TNFRSF14, and B7-H7. In certain embodiments the protein of the B7 family is PD-L1.

In certain embodiments, the variable domain that specifically binds to an extracellular domain of a protein of the B7 family blocks the binding of PD-L1 to its receptor. In certain embodiments, the receptor is PD-1 and/or CD80.

In certain embodiments, the PD-L1 binding domain of a multispecific antibody or variant thereof of the present disclosure comprises a heavy chain variable region, wherein the heavy chain variable region comprises the heavy chain CDR1 (HCDR1), heavy chain CDR2 (HCDR2), and heavy chain CDR3 (HCDR3) of one of the heavy chain variable regions having an amino acid sequence as set forth in SEQ ID NO: 20-24. The HCDRs according to Kabat are indicated in bold and underlined in the list of sequences provided herein.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20;
heavy chain CDR1 (HCDR1), heavy chain CDR2 (HCDR2), and heavy chain CDR3 (HCDR3), of a heavy chain variable region having an amino acid sequence as set forth in SEQ ID NO: 21;
heavy chain CDR1 (HCDR1), heavy chain CDR2 (HCDR2), and heavy chain CDR3 (HCDR3), of a heavy chain variable region having an amino acid sequence as set forth in SEQ ID NO: 22;
heavy chain CDR1 (HCDR1), heavy chain CDR2 (HCDR2), and heavy chain CDR3 (HCDR3), of a heavy chain variable region having an amino acid sequence as set forth in SEQ ID NO: 23; or
heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively;
heavy chain CDR1 (HCDR1), heavy chain CDR2 (HCDR2), and heavy chain CDR3 (HCDR3), having an amino acid sequence as set forth in SEQ ID NO: 110, SEQ ID NO: 111 and SEQ ID NO: 112, respectively;
heavy chain CDR1 (HCDR1), heavy chain CDR2 (HCDR2), and heavy chain CDR3 (HCDR3), having an amino acid sequence as set forth in SEQ ID NO: 113, SEQ ID NO: 114 and SEQ ID NO: 115, respectively;
heavy chain CDR1 (HCDR1), heavy chain CDR2 (HCDR2), and heavy chain CDR3 (HCDR3), having an amino acid sequence as set forth in SEQ ID NO: 107, SEQ ID NO: 116 and SEQ ID NO: 109, respectively; or
heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20; or
heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively; or
heavy chain CDR1 (HCDR1), heavy chain CDR2 (HCDR2), and heavy chain CDR3 (HCDR3), of a heavy chain variable region having an amino acid sequence as set forth in SEQ ID NO: 107, SEQ ID NO: 117 and SEQ ID NO: 109, respectively,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively,
wherein each of the HCDRs may comprise at most three, two, or one amino acid substitution.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20;
heavy chain CDR1 (HCDR1), heavy chain CDR2 (HCDR2), and heavy chain CDR3 (HCDR3), of a heavy chain variable region having an amino acid sequence as set forth in SEQ ID NO: 21;
heavy chain CDR1 (HCDR1), heavy chain CDR2 (HCDR2), and heavy chain CDR3 (HCDR3), of a heavy chain variable region having an amino acid sequence as set forth in SEQ ID NO: 22;
heavy chain CDR1 (HCDR1), heavy chain CDR2 (HCDR2), and heavy chain CDR3 (HCDR3), of a heavy chain variable region having an amino acid sequence as set forth in SEQ ID NO: 23; or
heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively;
heavy chain CDR1 (HCDR1), heavy chain CDR2 (HCDR2), and heavy chain CDR3 (HCDR3), having an amino acid sequence as set forth in SEQ ID NO: 110, SEQ ID NO: 111 and SEQ ID NO: 112, respectively;
heavy chain CDR1 (HCDR1), heavy chain CDR2 (HCDR2), and heavy chain CDR3 (HCDR3), having an amino acid sequence as set forth in SEQ ID NO: 113, SEQ ID NO: 114 and SEQ ID NO: 115, respectively;
heavy chain CDR1 (HCDR1), heavy chain CDR2 (HCDR2), and heavy chain CDR3 (HCDR3), having an amino acid sequence as set forth in SEQ ID NO: 107, SEQ ID NO: 116 and SEQ ID NO: 109, respectively; or
heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20; or
heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively; or
heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), of a heavy chain variable region having an amino acid sequence as set forth in SEQ ID NO: 20.

In certain embodiments, the binding domain that binds to PD-L1 comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively.

In certain embodiments, the PD-L1 binding domain of the multispecific antibody or variant thereof comprises a heavy chain variable region having an amino acid sequence as set forth in any one of SEQ ID NO: 20-24, or having at least 80%, preferably 85%, more preferably 90%, or most preferably 95% sequence identity thereto.

In certain embodiments, a PD-L1 binding domain of the multispecific antibody or variant thereof includes PD-L1 binding domain variants, wherein each of the HCDRs may comprise at most three, two, or one amino acid substitution. Such variants are expected to retain PD-L1 binding specificity.

In certain embodiments, a PD-L1 binding domain of the multispecific antibody or variant thereof also includes PD-L1 binding domain variants, which, in addition to variations in the HCDRs, comprise one or more variations in the framework regions. In certain embodiments, a PD-L1 binding domain variant of the multispecific antibody or variant thereof comprises no variations in the CDR regions but comprises one or more variations in the framework regions. Such variants have at least 80%, preferably 85%, more preferably 90%, or most preferably 95% sequence identity to the sequences disclosed herein, and are expected to retain PD-L1 binding specificity. Thus, in certain embodiments, a PD-L1 binding domain of the present disclosure comprises:
a heavy chain variable region having at least 80%, preferably 85%, more preferably 90%, or most preferably 95% sequence identity to the amino acid sequence as set forth in SEQ ID NO: 20, which heavy chain variable region comprises the HCDR1, HCDR2, and HCDR3 of the amino acid sequence as set forth in SEQ ID NO: 20;
a heavy chain variable region having at least 80%, preferably 85%, more preferably 90%, or most preferably 95% sequence identity to the amino acid sequence as set forth in SEQ ID NO: 21, which heavy chain variable region comprises the HCDR1, HCDR2, and HCDR3 of the amino acid sequence as set forth in SEQ ID NO: 21;
a heavy chain variable region having at least 80%, preferably 85%, more preferably 90%, or most preferably 95% sequence identity to the amino acid sequence as set forth in SEQ ID NO: 22, which heavy chain variable region comprises the HCDR1, HCDR2, and HCDR3 of the amino acid sequence as set forth in SEQ ID NO: 22;
a heavy chain variable region having at least 80%, preferably 85%, more preferably 90%, or most preferably 95% sequence identity to the amino acid sequence as set forth in SEQ ID NO: 23, which heavy chain variable region comprises the HCDR1, HCDR2, and HCDR3 of the amino acid sequence as set forth in SEQ ID NO: 23; or
a heavy chain variable region having at least 80%, preferably 85%, more preferably 90%, or most preferably 95% sequence identity to the amino acid sequence as set forth in SEQ ID NO: 24, which heavy chain variable region comprises the HCDR1, HCDR2, and HCDR3 of the amino acid sequence as set forth in SEQ ID NO: 24.

In certain embodiments, a PD-L1 binding domain of the present disclosure comprises:
a heavy chain variable region having at least 80%, preferably 85%, more preferably 90%, or most preferably 95% sequence identity to the amino acid sequence as set forth in SEQ ID NO: 20, which heavy chain variable region comprises the HCDR1, HCDR2, and HCDR3 amino acid sequence as set forth in SEQ ID NO: 107, SEQ ID NO: 108 and SEQ ID NO: 109, respectively;
a heavy chain variable region having at least 80%, preferably 85%, more preferably 90%, or most preferably 95% sequence identity to the amino acid sequence as set forth in SEQ ID NO: 21, which heavy chain variable region comprises the HCDR1, HCDR2, and HCDR3 amino acid sequence as set forth in SEQ ID NO: 110, SEQ ID NO: 111 and SEQ ID NO: 112, respectively;
a heavy chain variable region having at least 80%, preferably 85%, more preferably 90%, or most preferably 95% sequence identity to the amino acid sequence as set forth in SEQ ID NO: 22, which heavy chain variable region comprises the HCDR1, HCDR2, and HCDR3 amino acid sequence as set forth in SEQ ID NO: 113, SEQ ID NO: 114 and SEQ ID NO: 115, respectively;
a heavy chain variable region having at least 80%, preferably 85%, more preferably 90%, or most preferably 95% sequence identity to the amino acid sequence as set forth in SEQ ID NO: 23, which heavy chain variable region comprises the HCDR1, HCDR2, and HCDR3 amino acid sequence as set forth in SEQ ID NO: 107, SEQ ID NO: 116 and SEQ ID NO: 109, respectively; or
a heavy chain variable region having at least 80%, preferably 85%, more preferably 90%, or most preferably 95% sequence identity to the amino acid sequence as set forth in SEQ ID NO: 24, which heavy chain variable region comprises the HCDR1, HCDR2, and HCDR3 amino acid sequence as set forth in SEQ ID NO: 107, SEQ ID NO: 117 and SEQ ID NO: 109, respectively.

In certain embodiments, a PD-L1 binding domain of the multispecific antibody or variant thereof further comprises a light chain variable region. An example of a suitable light chain variable region is a light chain variable region as described herein. Light chain variable regions of PD-L1 antibodies available in the art may be used, or any other light chain variable region that can readily be obtained, such as from, for instance, an antibody display library by showing antigen binding activity when paired with a PD-L1 binding domain of the present disclosure. Preferably, a PD-L1 binding domain of the present disclosure comprises a VK1-39/JK1, VK1-39/JK5, VK3-15/JK1, VK3-20/JK1, or VL3-21/JL3 light chain variable region.

In certain embodiments, the PD-L1 binding domain of the multispecific antibody or variant thereof may further comprise a CH1 and CL region. Any CH1 domain may be used, in particular a human CH1 domain. An example of a suitable CH1 domain is provided by the amino acid sequence provided as SEQ ID NO: 42. Any CL domain may be used, in particular a human CL. An example of a suitable CL domain is provided by the amino acid sequence provided as SEQ ID NO: 43.

In certain embodiments, a LAG-3 binding domain disclosed herein can be combined with any PD-L1 binding domain disclosed herein to produce a multispecific antibody or variant thereof of the present disclosure. In certain embodiments, the present disclosure provides multispecific antibodies PB1-PB125, or variants thereof, as presented in Table 1.

TABLE 1
Binding moieties comprising combinations of heavy chain
variable regions specific for LAG-3 and heavy chain
variable regions specific for PD-L1. Each of PB1-PB125
can be combined with the light chain disclosed herein.

| | SEQ ID | SEQ ID | SEQ ID | SEQ ID | SEQ ID |
|---|---|---|---|---|---|
| | NO: 21 | NO: 22 | NO: 23 | NO: 24 | NO: 20 |
| SEQ ID NO: 1 | PB1 | PB2 | PB3 | PB4 | PB69 |
| SEQ ID NO: 2 | PB5 | PB6 | PB7 | PB8 | PB70 |
| SEQ ID NO: 3 | PB9 | PB10 | PB11 | PB12 | PB71 |
| SEQ ID NO: 4 | PB13 | PB14 | PB15 | PB16 | PB72 |
| SEQ ID NO: 5 | PB17 | PB18 | PB19 | PB20 | PB73 |
| SEQ ID NO: 6 | PB21 | PB22 | PB23 | PB24 | PB74 |
| SEQ ID NO: 7 | PB25 | PB26 | PB27 | PB28 | PB75 |
| SEQ ID NO: 8 | PB29 | PB30 | PB31 | PB32 | PB76 |
| SEQ ID NO: 9 | PB33 | PB34 | PB35 | PB36 | PB77 |
| SEQ ID NO: 10 | PB37 | PB38 | PB39 | PB40 | PB78 |
| SEQ ID NO: 11 | PB41 | PB42 | PB43 | PB44 | PB79 |
| SEQ ID NO: 12 | PB45 | PB46 | PB47 | PB48 | PB80 |
| SEQ ID NO: 13 | PB49 | PB50 | PB51 | PB52 | PB81 |
| SEQ ID NO: 14 | PB53 | PB54 | PB55 | PB56 | PB82 |
| SEQ ID NO: 15 | PB57 | PB58 | PB59 | PB60 | PB83 |
| SEQ ID NO: 16 | PB61 | PB62 | PB63 | PB64 | PB84 |
| SEQ ID NO: 17 | PB65 | PB66 | PB67 | PB68 | PB85 |
| SEQ ID NO: 52 | PB86 | PB87 | PB88 | PB89 | PB90 |
| SEQ ID NO: 53 | PB91 | PB92 | PB93 | PB94 | PB95 |
| SEQ ID NO: 54 | PB96 | PB97 | PB98 | PB99 | PB100 |
| SEQ ID NO: 55 | PB101 | PB102 | PB103 | PB104 | PB105 |
| SEQ ID NO: 56 | PB106 | PB107 | PB108 | PB109 | PB110 |
| SEQ ID NO: 57 | PB111 | PB112 | PB113 | PB114 | PB115 |
| SEQ ID NO: 58 | PB116 | PB117 | PB118 | PB119 | PB120 |
| SEQ ID NO: 59 | PB121 | PB122 | PB123 | PB124 | PB125 |

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 1; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 3; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24. In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 6; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 15; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 16; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 52; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 53; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 54; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 55; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 56; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 57; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 58; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 59; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20, 23 or 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 53; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 54; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 55; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 56; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 57; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 58; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 59; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 52; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively. In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 17; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 66, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 53; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24, wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 119, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 54; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 120, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 55; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 121, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 56; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 122, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 57; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 123, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 58; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 124, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 59; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 64, SEQ ID NO: 65 and SEQ ID NO: 125, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 117 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 5; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 72, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 52; and
a PD-L1 binding domain of the present disclosure comprising heavy chain CDR1 (HCDR1), heavy chain CDR2 (HCDR2), and heavy chain CDR3 (HCDR3), of a heavy chain variable region having an amino acid sequence as set forth in SEQ ID NO: 20,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 70, SEQ ID NO: 71 and SEQ ID NO: 118, respectively; and
a PD-L1 binding domain of the present disclosure comprising heavy chain CDR1 (HCDR1), heavy chain CDR2 (HCDR2), and heavy chain CDR3 (HCDR3), having an amino acid sequence as set forth in SEQ ID NO: 107, SEQ ID NO: 108 and SEQ ID NO: 109, respectively,
wherein the LAG-3 binding domain and PD-L1 binding domain comprise light chain CDR1 (LCDR1) having an amino acid sequence as set forth in SEQ ID NO: 31, light chain CDR2 (LCDR2) having an amino acid sequence as set forth in SEQ ID NO: 32, and light chain CDR3 (LCDR3) having an amino acid sequence as set forth in SEQ ID NO: 33.

In one embodiment, the multispecific antibody or variant thereof comprises:
a LAG-3 binding domain of the present disclosure comprising a heavy chain variable region having an amino acid sequence as set forth in SEQ ID NO: 17; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 53; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 54; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 55; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 56; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 57; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 58; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 59; and
a PD-L1 binding domain of the present disclosure comprising a heavy chain variable region having an amino acid sequence as set forth in SEQ ID NO: 24.

In one embodiment, the multispecific antibody or variant thereof comprises:
a LAG-3 binding domain of the present disclosure comprising a heavy chain variable region having an amino acid sequence as set forth in SEQ ID NO: 5; and
a PD-L1 binding domain of the present disclosure comprising a heavy chain variable region having an amino acid sequence as set forth in SEQ ID NO: 20.

In one embodiment, the multispecific antibody or variant thereof comprises:
a LAG-3 binding domain of the present disclosure comprising a heavy chain variable region having an amino acid sequence as set forth in SEQ ID NO: 52; and
a PD-L1 binding domain of the present disclosure comprising a heavy chain variable region having an amino acid sequence as set forth in SEQ ID NO: 20. In one embodiment, the multispecific antibody or variant thereof comprises:
a LAG-3 binding domain of the present disclosure comprising a heavy chain variable region having an amino acid sequence as set forth in SEQ ID NO: 17; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 53; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 54; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 55; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 56; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 57; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 58; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 59; and
a PD-L1 binding domain of the present disclosure comprising a heavy chain variable region having an amino acid sequence as set forth in SEQ ID NO: 24,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 5; and
a PD-L1 binding domain of the present disclosure comprising a heavy chain variable region having an amino acid sequence as set forth in SEQ ID NO: 20,
wherein the LAG-3 binding domain and PD-L1 binding domain comprise a light chain variable region having an amino acid sequence as set forth in SEQ ID NO: 30.

In one embodiment, the multispecific antibody or variant thereof comprises:
a LAG-3 binding domain of the present disclosure comprising a heavy chain variable region having an amino acid sequence as set forth in SEQ ID NO: 52; and
a PD-L1 binding domain of the present disclosure comprising a heavy chain variable region having an amino acid sequence as set forth in SEQ ID NO: 20,
wherein the LAG-3 binding domain and PD-L1 binding domain comprise a light chain variable region having an amino acid sequence as set forth in SEQ ID NO: 30.

A LAG-3×PD-L1 multispecific antibody or variant thereof of the present disclosure targets two immune checkpoints in trans: it binds to PD-L1 expressed on tumor cells or antigen-presenting cells and, simultaneously, LAG-3 expressed on T-cells. It could also function in cis by binding to PD-L1 upregulated on exhausted T-effector cells in the tumor microenvironment. In this way, a LAG-3×PD-L1 multispecific antibody or variant thereof of the present disclosure prevents T-cells inhibitory signaling and enhances tumor immunity.

In certain embodiments, the multispecific antibody or variant thereof has a binding affinity for human LAG-3 in a range of about 1-2 nM, in particular in a range of about 1.45-1.93 nM, as measured by SPR as described herein. The term “about” allows for a deviation of 10% from the stated values.

In certain embodiments, the multispecific antibody or variant thereof has a binding affinity for human LAG-3 in a range of 1-2 nM, in particular in a range of 1.45-1.93 nM, as measured by SPR as described herein.

In certain embodiments, the multispecific antibody or variant thereof has a binding affinity for human PD-L1 in a range of 0.1-0.5 nM, in particular in a range of 0.17-0.41 nM, as measured by SPR as described herein.

In certain embodiments, the present disclosure provides a multispecific antibody comprising a binding domain that binds to LAG-3 and a binding domain that binds to PD-L1, or a variant thereof that maintains the binding specificity of the antibody, wherein the antibody or variant has a higher affinity for cynomolgus LAG-3 than a reference antibody comprising two heavy chain variable regions having an amino acid sequence as set forth in SEQ ID NO: 27 and two light chain variable regions having an amino acid sequence as set forth in SEQ ID NO: 28, and a higher affinity for cynomolgus PD-L1 than a reference antibody comprising two heavy chain variable regions having an amino acid sequence as set forth in SEQ ID NO: 25 and two light chain variable regions having an amino acid sequence as set forth in SEQ ID NO: 26.

Determining if a multispecific antibody or variant thereof has a higher binding affinity than a reference antibody can be done by measuring the binding affinity of both the multispecific antibody or variant thereof and the reference antibody in the same type of assay, using the same assay conditions. Thus, in certain embodiments, the binding affinity of the multispecific antibody or variant thereof and the binding affinity of the reference antibody are measured in the same type of assay, using the same assay conditions. In certain embodiments, the assay is an assay that uses surface plasmon resonance (SPR). SPR is an assay that uses surface plasmon resonance (SPR) to measure binding affinity, such as the biosensor system of Biacore®, or Solution Equilibrium Titration (SET) (see Friguet B et al. (1985) J. Immunol Methods; 77(2): 305-319, and Hanel C et al. (2005) Anal Biochem; 339(1): 182-184).

The binding affinity values of the LAG-3 and PD-L1 binding domains as provided herein are obtained with the method described in Example 3. In brief, anti-huIgG Fcγ is covalently coupled to the surface of a CM5 sensor chip using free amine chemistry. Multispecific antibodies are injected at concentrations up to 20 nM at 30 μL/min for 2 minutes in the flow cell. Subsequently, antigen (0.6-20 nM) is flowed over the surface of the CM5 sensor chip at 30 μL/min for 2 minutes. Sensorgrams of the association and dissociation phases for the different antigens are obtained. Using the BIA evaluation software and curve-fitting employing a 1:1 interaction model (for monovalent interaction), the affinities of the individual Fab arms are determined.

In certain embodiments, the binding affinity is measured with the LAG-3×PD-L1 multispecific antibody of the present disclosure in bivalent bispecific format and the reference anti-human LAG-3 antibody in bivalent monospecific IgG format. The binding affinity of the multispecific antibody for human LAG-3 thus represents a monovalent binding affinity.

In certain embodiments, the binding affinity is measured with the LAG-3×PD-L1 multispecific antibody of the present disclosure in bivalent bispecific format and the reference anti-human PD-L1 antibody in bivalent monospecific IgG format. The binding affinity of the multispecific antibody for human PD-L1 thus represents a monovalent binding affinity.

In certain embodiments, the LAG-3 binding domain of the multispecific antibody or variant thereof has at least a ten-fold, preferably a ten to twenty fold, higher binding affinity for cynomolgus LAG-3 than the reference anti-human LAG-3 binding domain as described herein, as measured by SPR as described herein.

In certain embodiments, the LAG-3 binding domain of the multispecific antibody or variant thereof has a ten-fold higher binding affinity for cynomolgus LAG-3 than the reference anti-human LAG-3 binding domain as described herein, as measured by SPR as described herein.

In certain embodiments, the PD-L1 binding domain of the multispecific antibody or variant has at least a ten-fold higher, preferably a ten to fifty fold, in particular a ten to forty, ten to thirty, ten to twenty, fold, higher binding affinity for cynomolgus PD-L1 than the reference anti-human PD-L1 binding domain as described herein, as measured by SPR as described herein.

In certain embodiments, the LAG-3 binding domain of the multispecific antibody or variant has a binding affinity for cynomolgus LAG-3 in a range of about 0.1-2 nM, in particular in a range of about 0.3-1.5 nM; about 0.35-1.5 nM or about 0.4-1.2 nM, more in particular in a range of about 0.41-1.15 nM, as measured by SPR as described herein. The term “about” allows for a deviation of 10% from the stated values.

In certain embodiments, the LAG-3 binding domain of the multispecific antibody or variant has a binding affinity for cynomolgus LAG-3 in a range of 0.1-2 nM, in particular in a range of 0.3-1.5 nM; 0.35-1.5 nM or 0.4-1.2 nM, more in particular in a range of 0.41-1.15 nM, as measured by SPR as described herein.

In certain embodiments, the PD-L1 binding domain of the multispecific antibody or variant has a binding affinity for cynomolgus PD-L1 in a range of about 0.05-1 nM, in particular in a range of about 0.05-0.5 nM; about 0.1-0.4 nM or about 0.1-0.35 nM, more in particular in a range of about 0.15-0.34 nM, as measured by SPR as described herein. The term “about” allows for a deviation of 10% from the stated values.

In certain embodiments, the PD-L1 binding domain of the multispecific antibody or variant has a binding affinity for cynomolgus PD-L1 in a range of 0.05-1 nM, in particular in a range of 0.05-0.5 nM; 0.1-0.4 nM or 0.1-0.35 nM, more in particular in a range of 0.15-0.34 nM, as measured by SPR as described herein.

In certain embodiments, the binding affinity for cynomolgus LAG-3 is measured with both the LAG-3 binding domain of the multispecific antibody and the reference anti-LAG-3 binding domain in a bivalent bispecific IgG format. A bivalent bispecific IgG format may for instance comprise a LAG-3 binding domain of the present disclosure or of the reference antibody and a binding domain that binds PD-L1 or another unrelated target. It is thus the monovalent interaction with cynomolgus LAG-3 that is being measured.

In certain embodiments, the binding affinity for cynomolgus PD-L1 is measured with both the PD-L1 binding domain of the multispecific antibody and the reference PD-L1 binding domain in a bivalent bispecific IgG format. A bivalent bispecific IgG format may for instance comprise a PD-L1 binding domain of the present disclosure or of the reference antibody and a binding domain that binds LAG-3 or another unrelated target. It is thus the monovalent interaction with cynomolgus PD-L1 that is being measured.

In certain embodiments, the multispecific antibody or variant thereof is capable of enhancing the proliferation of CD4⁺ and/or CD8⁺ tumor-infiltrating T cells. In certain embodiments, the multispecific antibody or variant thereof is capable of increasing the number of CD4⁺ and/or CD8⁺ T cells, in particular proliferating CD4⁺ and/or CD8⁺ T cells, in the tumor microenvironment. This can for instance be determined by measuring the number of CD4⁺ and/or CD8⁺ T cells, in particular proliferating CD4⁺ and/or CD8⁺ T cells, in a tumor biopsy, as described for instance in Example 4.

In certain embodiments, the multispecific antibody or variant thereof results in a reduced number of regulatory T cells in the tumor microenvironment as compared to the number of regulatory T cells in response to a combination of the reference antibodies as described herein.

In certain embodiments, the multispecific antibody or variant thereof is a full length antibody, in particular a full length bispecific antibody, more in particular a full length bispecific IgG1 antibody.

In certain embodiments, the present disclosure provides a vector comprising a nucleic acid sequence encoding the heavy chain variable region of a LAG-3 binding domain as described herein and a nucleic acid sequence encoding the heavy chain variable region of a PD-L1 binding domain as described herein. In certain embodiments, the vector further comprises a nucleic acid sequence encoding a CH1 region and preferably a hinge, CH2 and CH3 region. In certain embodiments, the vector further comprises at least one polynucleotide encoding a light chain variable region, and preferably a CL region. In certain embodiments, the light chain variable region is a light chain variable region of a light chain that is capable of pairing with multiple heavy chains having different epitope specificities.

The present disclosure further provides a cell comprising a nucleic acid sequence encoding the heavy chain variable region of a LAG-3 binding domain as described herein and a nucleic acid sequence encoding the heavy chain variable region of a PD-L1 binding domain as described herein. In certain embodiments, the cell may further comprise a nucleic acid sequence encoding a CH1 region and preferably a hinge, CH2 and CH3 region. In certain embodiments, the cell may further comprise at least one polynucleotide sequence encoding a light chain variable region, and preferably a CL region. In certain embodiments, the light chain variable region is a light chain variable region of a light chain that is capable of pairing with multiple heavy chains having different epitope specificities.

The present disclosure also provides a cell producing a multispecific antibody or variant thereof as described herein. In certain embodiments, the cell is a recombinant cell, which has been transformed with a vector as described herein.

Further provided herein is a method for producing a multispecific antibody or variant thereof of the present disclosure, wherein the method comprises culturing a cell as described herein and recovering the multispecific antibody or variant thereof, from the cell or supernatant.

Further provided herein is a method for producing a variant of a LAG-3 binding domain and/or a PD-L1 binding domain of the present disclosure, wherein the method comprises:
generating a sequence variant of a heavy chain variable region as described herein; and
expressing the sequence variant or variants and a light chain variable region as described herein in a cell.

Methods for generating sequence variants are well known in the art. One can take a random approach in generating sequence variants or a targeted approach, where one can for instance aim at introducing variations that are likely to increase or decrease binding affinity. Routine methods for affinity maturing antibody binding domains are widely known in the art, see for instance Tabasinezhad M. et al. Immunol Lett. 2019; 212:106-113. One can also aim at introducing variations that mitigate developability risks with a view on producing a binding domain, or multispecific antibody comprising such binding domain, at large scale. Variations may be introduced that are likely not to cause a loss in binding specificity and/or affect binding affinity. Permissive substitutions include, but are not limited to, those substitutions which result in similar biophysical properties of the variant, for example Isoleucine to Leucine or Valine, Threonine to Serine, Arginine to Lysine, Aspartate to Glutamate and Tryptophan to Tyrosine. Variations may also be introduced at certain positions based on frequency in germlines or natural repertoires.

Whether amino acid residues within the CDRs and/or framework regions can be substituted, for instance with a conservative amino acid residue, and without, or substantially without, loss in binding specificity and/or affinity, can be determined by methods well known in the art. Experimental examples include, but are not limited to, for instance, alanine scanning (Cunningham B C, Wells J A. Science. 1989; 244(4908):1081-5), and deep mutational scanning (Araya C L, Fowler D M. Trends Biotechnol. 2011; 29(9):435-42). Computational methods have also been developed that can predict the effect of amino acid variation, such as for instance described in Sruthi C K, Prakash M. PLoS One. 2020; 15(1):e0227621, Choi Y. et al. PLoS One. 2012; 7(10):e46688, and Munro D, Singh M. Bioinformatics. 2020; 36(22-23):5322-9.

Further provided herein are multispecific antibodies comprising any variant LAG-3 and/or PD-L1 binding domains produced by the above described method; a pharmaceutical composition comprising a multispecific antibody that comprises any of said variant LAG-3 and/or PD-L1 binding domains; nucleic acids encoding any of said variant binding domains; vectors and cells comprising said nucleic acids; and use of a multispecific antibody comprising any of said variant LAG-3 and/or PD-L1 binding domains or said pharmaceutical composition for the treatment of cancer.

### Pharmaceutical Composition and Methods

In certain embodiments, the present disclosure provides a pharmaceutical composition comprising the multispecific antibody or variant thereof as described herein and a pharmaceutical acceptable carrier, diluent or excipient.

In certain embodiments, the present disclosure provides a multispecific antibody or variant thereof, or pharmaceutical composition, as described herein for use as a medicament.

In certain embodiments, the present disclosure provides a multispecific antibody or variant thereof as described herein, or the pharmaceutical composition, as described herein, for use in the treatment of a disease associated with a suppressed immune system, in particular cancer.

In certain embodiments, the present disclosure provides a method for treating a disease, comprising administering an effective amount of the multispecific antibody or variant thereof, or the pharmaceutical composition, as described herein to an individual in need thereof.

In certain embodiments, the present disclosure provides a method for treating a disease associated with a suppressed immune system, in particular cancer, comprising administering an effective amount of the multispecific antibody or variant thereof, or the pharmaceutical composition, as described herein to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence.

The effective amount of the agent or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual.

An effective amount can be administered in one or more administrations.

A therapeutic amount also includes an amount that balances any toxic or detrimental effects of the agent or combination of agents and the therapeutically beneficial effects.

The term “agent” refers to a therapeutically active substance, in the present case a multispecific antibody or variant thereof of the present disclosure, or a pharmaceutical composition of the present disclosure.

### General Terms

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. The antigen binding site binds the antigen and a variety of variants thereof, such as those expressed by some antigen positive immune or tumor cells.

When herein reference is made to a gene, a protein, the reference is preferably to the human form of the gene or protein. When herein reference is made to a gene or protein reference is made to the natural gene or protein and to variant forms of the gene or protein as can be detected in tumors, cancers and the like, preferably as can be detected in human tumors, cancers and the like.

HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensemble provides the accession number with which information on the gene or protein encoded by the gene can be obtained from the Ensemble database. Ensembl is a joint project between EMBL-EBI and the Wellcome Trust Sanger Institute to develop a software system which produces and maintains automatic annotation on selected eukaryotic genomes.

### BRIEF DESCRIPTION OF THE DRAWINGS

In the Figures, bivalent monospecific antibodies are indicated in the format SEQ ID NO: A, where SEQ ID NO: A refers to the heavy chain variable sequence of both binding domains. Each binding domain of the monospecific antibodies comprises a light chain. In the Examples, which are used to illustrate the present disclosure but are not intended to limit the disclosure in any way, each binding domain of the monospecific antibodies comprises a light chain variable region variable region having an amino acid sequence as set forth in SEQ ID NO: 30 and a light chain constant region having an amino acid sequence as set forth in SEQ ID NO: 43. The monospecific antibodies preferably are IgG1 antibodies comprising a CH1, hinge, CH2, and CH3. In the Examples, which are used to illustrate the present disclosure but are not intended to limit the disclosure in any way, monospecific antibodies were screened in IgG1 format, wherein the LAG-3 and PD-L1 binding heavy chains comprise a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 47.

Bispecific antibodies are indicated in the format SEQ ID NO: A×SEQ ID NO: B, where both SEQ ID NO: A and B refer to heavy chain variable sequences. Each binding domain of the bispecific antibodies comprises a light chain. In the Examples, which are used to illustrate the present disclosure but are not intended to limit the disclosure in any way, each binding domain of the bispecific antibodies comprises a light chain variable region variable region having an amino acid sequence as set forth in SEQ ID NO: 30 and a light chain constant region having an amino acid sequence as set forth in SEQ ID NO: 43. The bispecific antibodies are IgG1 antibodies, comprising a CH1, hinge, CH2, and CH3.

In Example 1, which is used to illustrate the present disclosure but is not intended to limit the disclosure in any way, bispecific antibodies were screened in IgG1 format, wherein the PD-L1 binding heavy chain comprises a CH1 having an amino acid sequence as set forth in SEQ ID NO: 42, a CH2 that may have an amino acid sequence as set forth in SEQ ID NO: 45, and a CH3 that may have an amino acid sequence as set forth in SEQ ID NO: 50; and the LAG-3 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 51.

In Example 2, which is used to illustrate the present disclosure but is not intended to limit the disclosure in any way, bispecific antibodies were screened in IgG1 format, wherein the PD-L1 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 50; and the LAG-3 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 51.

In Examples 3 and 4, which are used to illustrate the present disclosure but are not intended to limit the disclosure in any way, bispecific antibodies were screened in IgG1 format.

For SEQ ID NO: 17×SEQ ID NO:24, the PD-L1 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 50; and the LAG-3 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 51.

For SEQ ID NO: 5×SEQ ID NO: 20, the PD-L1 binding heavy chain may comprise a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 51; and the LAG-3 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 50.

In Example 5, which is used to illustrate the present disclosure but is not intended to limit the disclosure in any way, bispecific antibodies were screened in IgG format, wherein the PD-L1 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 50 or 51; and the LAG-3 binding heavy chain comprises a CH1 having an amino acid sequence as set forth in SEQ ID NO: 42, a CH2 having an amino acid sequence as set forth in SEQ ID NO: 45, and a CH3 having an amino acid sequence as set forth in SEQ ID NO: 51 or 50, as indicated in (Table 10). Bivalent monospecific relatlimab analog antibody and bivalent monospecific atezolizumab analog antibody are indicated in the format SEQ ID NO: A/SEQ ID NO: B, where SEQ ID NO: A refers to the respective heavy chain sequence and SEQ ID NO: B refers to the respective light chain sequence. Bivalent monospecific relatlimab analog antibody comprises two LAG-3 binding domains. Bivalent monospecific atezolizumab analog antibody comprises two PD-L1 binding domains. Each binding domain of the analog antibodies comprises a light chain. A combination of relatlimab and atezolizumab analogs is indicated in the format SEQ ID NO: A/SEQ ID NO: B+SEQ ID NO: C/SEQ ID NO: D, where SEQ ID NO: A refers to the heavy chain sequence and SEQ ID NO: B refers to the light chain sequence of either relatlimab or atezolizumab analog, and SEQ ID NO: C to the heavy chain sequence and SEQ ID NO: D to the light chain sequence of the other.

FIG. 1A shows the results of LAG-3 binding using FACS. Bispecific antibodies comprising a heavy chain variable region having an amino acid sequence as set forth in SEQ ID NO: 1 and SEQ ID NO: 23 (a), SEQ ID NO: 3 and SEQ ID NO: 23 (b), SEQ ID NO: 5 and SEQ ID NO: 23 (c), SEQ ID NO: 6 and SEQ ID NO: 23 (d), SEQ ID NO: 15 and SEQ ID NO: 23 (e), and SEQ ID NO: 16 and SEQ ID NO: 23 (f) were compared with bivalent monospecific antibodies comprising the respective LAG-3 binding domains, positive control relatlimab analog (SEQ ID NO: 27/SEQ ID NO: 28), and a negative control antibody (SEQ ID NO: 29/SEQ ID NO: 30). Each graph shows the result for binding to human and rhesus LAG-3.

The X-axis shows the concentration of antibody in μg/mL. The Y-axis shows the level of binding expressed in Mean Fluorescence Intensity (MFI).

FIG. 1B shows the results of PD-L1 binding using FACS. Bispecific antibodies comprising a heavy chain variable region having an amino acid sequence as set forth in SEQ ID NO: 1 and SEQ ID NO: 23, SEQ ID NO: 3 and SEQ ID NO: 23, SEQ ID NO: 5 and SEQ ID NO: 23, SEQ ID NO: 6 and SEQ ID NO: 23, SEQ ID NO: 15 and SEQ ID NO: 23, and SEQ ID NO: 16 and SEQ ID NO: 23 were compared with a bivalent monospecific antibody comprising the PD-L1 binding domain having an amino acid sequence as set forth in SEQ ID NO: 23, positive control atezolizumab analog (SEQ ID NO: 25/SEQ ID NO: 26), and a negative control antibody (SEQ ID NO: 29/SEQ ID NO: 30). The top graph shows the result for binding to human PD-L1; the bottom graph shows the result for binding to rhesus PD-L1.

The X-axis shows the concentration of antibody in μg/mL. The Y-axis shows the level of binding expressed in Mean Fluorescence Intensity (MFI).

FIG. 2 shows the results from the PD-1/LAG-3 reporter assay. Bispecific antibodies comprising a heavy chain variable region having an amino acid sequence as set forth in SEQ ID NO: 1 and SEQ ID NO: 23, SEQ ID NO: 3 and SEQ ID NO: 23, SEQ ID NO: 5 and SEQ ID NO: 23, SEQ ID NO: 6 and SEQ ID NO: 23, SEQ ID NO: 15 and SEQ ID NO: 23, and SEQ ID NO: 16 and SEQ ID NO: 23 were compared with positive control atezolizumab analog (SEQ ID NO: 25/SEQ ID NO: 26), positive control relatlimab analog (SEQ ID NO: 27/SEQ ID NO: 28), a combination of atezolizumab and relatlimab analogs (SEQ ID NO: 25/SEQ ID NO:26+SEQ ID NO: 27/SEQ ID NO: 28), and a negative control antibody (SEQ ID NO: 29/SEQ ID NO: 30).

The X-axis shows the concentration of antibody in μg/mL. The Y-axis shows the fold induction.

FIG. 3 shows the results from the SEB assay from donor 1 (FIG. 3A) and donor 2 (FIG. 3B).

FIGS. 3A1 and 3B1 show a comparison in fold induction of IL-2 of bispecific antibodies comprising a heavy chain variable region having an amino acid sequence as set forth in SEQ ID NO: 1 and SEQ ID NO: 23, SEQ ID NO: 3 and SEQ ID NO: 23, SEQ ID NO: 5 and SEQ ID NO: 23, SEQ ID NO: 6 and SEQ ID NO: 23, SEQ ID NO: 15 and SEQ ID NO: 23, and SEQ ID NO: 16 and SEQ ID NO: 23 with positive control atezolizumab analog (SEQ ID NO: 25/SEQ ID NO: 26), a combination of atezolizumab and relatlimab analogs (SEQ ID NO: 25/SEQ ID NO:26+SEQ ID NO: 27/SEQ ID NO: 28), and a negative control antibody (SEQ ID NO: 29/SEQ ID NO: 30).

FIGS. 3A2 and 3B2 show a comparison in fold induction of TNFa of the same antibodies.

The X-axis shows the concentration of antibody in μg/mL. The Y-axis shows the fold induction of IL-2 or TNFa. Fold induction of each antibody was calculated relative to control wells containing no IgG.

FIG. 4 shows the binding affinity data of two bispecific antibodies.

FIG. 5 shows the results of two bispecific antibodies in FACS binding, PD-1/LAG-3 reporter, and SEB assays.

FIG. 5A compares the binding to human LAG-3 of the bispecific antibodies comprising a heavy chain variable region having an amino acid sequence as set forth in SEQ ID NO: 17 and SEQ ID NO: 24, and SEQ ID NO: 5 and SEQ ID NO: 20 with that of positive control relatlimab analog (SEQ ID NO: 27/SEQ ID NO:28) and a negative control antibody.

FIG. 5B compares the binding to rhesus LAG-3 of the same antibodies.

FIG. 5C compares the binding to human PD-L1 of the bispecific antibodies comprising a heavy chain variable region having an amino acid sequence as set forth in SEQ ID NO: 17 and SEQ ID NO: 24, and SEQ ID NO: 5 and SEQ ID NO: 20 with that of positive control atezolizumab analog (SEQ ID NO: 25/SEQ ID NO:26) and a negative control antibody.

FIG. 5D compares the binding to rhesus PD-L1 of the same antibodies.

The X-axis shows the concentration of antibody in μg/mL. The Y-axis shows the level of binding expressed in Mean Fluorescence Intensity (MFI).

FIG. 5E shows the results of the PD-1/LAG-3 reporter assay, wherein bispecific antibodies comprising a heavy chain variable region having an amino acid sequence as set forth in SEQ ID NO: 17 and SEQ ID NO: 24, and SEQ ID NO: 5 and SEQ ID NO: 20 are compared with positive controls atezolizumab analog (SEQ ID NO: 25/SEQ ID NO:26) and relatlimab analog (SEQ ID NO: 27/SEQ ID NO:28), a combination of relatlimab and atezolizumab analogs (SEQ ID NO: 27/SEQ ID NO:28+SEQ ID NO: 25/SEQ ID NO: 26, and a negative control antibody.

FIG. 5F shows the results of the SEB assay, comparing the fold induction of IL-2 of bispecific antibodies comprising a heavy chain variable region having an amino acid sequence as set forth in SEQ ID NO: 17 and SEQ ID NO: 24, and SEQ ID NO: 5 and SEQ ID NO: 20 with that of positive controls atezolizumab analog (SEQ ID NO: 25/SEQ ID NO:26) and relatlimab analog (SEQ ID NO: 27/SEQ ID NO:28), a combination of relatlimab and atezolizumab analogs (SEQ ID NO: 27/SEQ ID NO:28+SEQ ID NO: 25/SEQ ID NO: 26, and a negative control antibody, in four different donors. The four graphs represent the results of four different donors. The X-axis shows the concentration of antibody in μg/mL. The Y-axis shows the concentration of IL-2 in pg/mL.

FIG. 6 shows the data from the an in vivo study. The effect on tumor volume of hu-CD34 NSG™ mice with MDA-MB-231 tumors was assessed for bispecific antibodies comprising a heavy chain variable region having an amino acid sequence as set forth in SEQ ID NO: 17 and SEQ ID NO: 24, and SEQ ID NO: 5 and SEQ ID NO: 20, and compared with atezolizumab analog (Atezolizumab*), relatlimab analog (Relatlimab*, and a combination of relatlimab and atezolizumab analogs (Relatlimab*+Atezolizumab*).

FIG. 6A1 shows the data from mice immunized with CD34⁺ HSC from donor 1, FIG. 6A2 from mice immunized with CD34⁺ HSC from donor 2, and FIG. 6A3 from mice immunized with CD34⁺ HSC from donor 3. FIG. 6A4 shows the data from all donors combined.

FIG. 6B shows the results from the analysis of CD4⁺ and CD8⁺ T cell and regulatory T cell (Treg) populations in MDA-MB-231 tumor samples. The effect on the percentage CD3+ T cells (FIG. 6B1), CD8+ T cells (FIG. 6B2), FOXP3+ regulatory T cells (FIG. 6B3), proliferating CD8+ T cells (FIG. 6B4), and proliferating CD4+ T-cells (FIG. 6B5), was assessed for bispecific antibodies comprising a heavy chain variable region having an amino acid sequence as set forth in SEQ ID NO: 17 and SEQ ID NO: 24, and SEQ ID NO: 5 and SEQ ID NO: 20, and compared with atezolizumab analog, relatlimab analog, and a combination of relatlimab and atezolizumab analogs.

FIG. 7 shows the results of a binding assay using FACS. Bispecific antibodies comprising a heavy chain variable region having an amino acid sequence as set forth in Table 10 were tested along with RSV control antibody (SEQ ID NO: 29/SEQ ID NO: 30). The X-axis shows the concentration of antibody in μg/mL. The Y-axis shows fold induction obtained by normalizing mean MFI values with respect to that of wells without antibody.

FIG. 7A shows the results of binding to human LAG-3.

FIG. 7B shows the results of binding to rhesus LAG-3.

FIG. 7C shows the results of binding to human PD-L1.

FIG. 8 shows the results from a PD-1/LAG-3 reporter assay. Bispecific antibodies comprising a heavy chain variable region having an amino acid sequence as set forth in Table 10 were compared with a combination of atezolizumab and relatlimab analogs (SEQ ID NO: 25/SEQ ID NO:26+SEQ ID NO: 27/SEQ ID NO: 28), and RSV control antibody (SEQ ID NO: 29/SEQ ID NO: 30).

The X-axis shows the concentration of antibody in μg/mL. The Y-axis shows the fold induction.

The following Examples illustrate the present disclosure but are not intended to limit the disclosure in any way.

### EXAMPLES

### Example 1—Production and Identification of LAG-3×PD-L1 Bispecific Antibodies

Anti-human LAG-3 binding domains can be obtained by methods known in the art, such as for instance as described in WO 2019/009728. A large panel of heavy chain variable regions were obtained by immunizing transgenic mice comprising a common IGKV1-39 light chain (MeMo® mice) with human LAG-3 antigenic moieties, including the use of different forms of DNA, protein and cell-based antigen delivery.

Anti-human PD-L1 binding domains can be obtained by methods known in the art, such as for instance as described in WO 2018/056821 and WO 2019/009726. A large panel of heavy chain variable regions were obtained by immunizing transgenic mice comprising a common IGKV1-39 light chain (MeMo® mice) with human PD-L1 antigenic moieties, including the use of different forms of DNA, protein and cell-based antigen delivery.

A number of anti-human LAG-3 Fabs were selected, the heavy chain variable regions of which were used for the production of bispecific anti-LAG-3×anti-PD-L1 antibodies. The amino acid sequences of these heavy chain variable regions are set forth in SEQ ID NOs: 1-16. Fabs comprising these heavy chain variable regions bind domain 1 or domain 2 of human LAG-3 and showed functional activity in a LAG-3 reporter assay (data not shown). Two additional Fabs were selected, the heavy chain variable regions of which comprise the amino acid sequences as set forth in SEQ ID Nos: 18 and 19. Fabs comprising these heavy chain variable regions bind domain 3 and domain 4 of human LAG-3. These two Fabs did not show functional activity in the LAG-3 reporter and were selected for the production of negative control bispecific antibodies.

A number of anti-human PD-L1 Fabs were selected, the heavy chain variable regions of which were used for the production of the bispecific antibodies. The amino acid sequences of these heavy chain variable regions are as set forth in SEQ ID NOs: 21-23. Fabs comprising these heavy chain variable regions block binding of PD-L1 to PD-1 (data not shown). The heavy chain variable domains exhibit different affinities as determined in a bispecific IgG format comprising one PD-L1 binding Fab and one other Fab (binding tetanus toxoid (TT) or anti-PD-1), as measured using SPR. It is thus the binding affinity of a monovalent interaction with PD-L1 that has been measured.

The affinity for PD-L1 of the anti-PD-L1 Fab arms was determined using surface plasmon resonance (SPR). Affinity was measured in bispecific IgG format, having just one arm specific for PD-L1. To determine the kinetics of binding of anti-PD-L1 Fab arms to the antigen, Surface Plasmon Resonance (SPR) using a BIAcore T100 was used. Recombinant, purified, Fc-tagged human PD-L1 (R&D Systems, cat. nr. 156-B7-100) was coupled to flow cell (FC) 2 of a CM5 sensor chip (FC1 served as blank for subtraction and was activated, then inactivated directly using ethanolamine) at to a level of approximately 200 resonance units (RU) using NHS/EDC chemistry at pH5.0 (NaAc buffer), 2 μg/mL antigen concentration and 10 μL/min flow rate. Bispecific IgG composed of an anti-PD-L1 Fab arm and an irrelevant Fab arm were then run over the surfaces of FC1 and 2 at different concentrations (100 nM and serial 2-fold dilutions in HBS, 6 dilutions) in a kinetic run at 30 μL/min. Regeneration was performed using a pulse of 50 mM HCl in water (15 μL at a flow rate of 10 μL/min). Obtained sensorgrams were evaluated using the BIAevaluation software and kinetic association- and dissociation rate constants were determined. Several measurements were performed on different surfaces of different sizes on several days. Different measurements gave very similar results, underscoring their validity. All measurements were carried out at 25° C.

The binding affinities are presented in Table 2.

TABLE 2

| IgG comprising: | Binding affinity (nM) |
|---|---|
| SEQ ID NO: 21 | 2.5 |
| SEQ ID NO: 22 | 5.5 |
| SEQ ID NO: 23 | 0.6 |

Bispecific IgG antibodies were generated by transient co-transfection of two plasmid vectors: one encoding an IgG heavy chain with a LAG-3 binding VH region and the other encoding an IgG heavy chain with a PD-L1 binding VH region. CH3 engineering technology as described in WO 2013/157954 and WO 2013/157953 was employed to ensure efficient hetero-dimerization and formation of bispecific antibodies. Both vectors further encode a common light chain comprising the IGKV1-39/Jk1 light chain variable region. Cell transfection, cell culture, and the harvesting and purification of antibodies was performed by methods known in the art.

### Example 2—Screening of LAG-3×PD-L1 Bispecific Antibodies

### Reference and Control Antibodies

Bispecific antibodies were compared with an analog of anti-LAG-3 antibody relatlimab, which comprises two heavy chain variable regions having an amino acid sequence as set forth in SEQ ID NO: 27 and two light chain variable regions having an amino acid sequence as set forth in SEQ ID NO: 28 (relatlimab analog), and an analog of anti-PD-L1 antibody atezolizumab, which comprises two heavy chain variable regions having an amino acid sequence as set forth in SEQ ID NO: 25 and two light chain variable regions having an amino acid sequence as set forth in SEQ: 26 (atezolizumab analog), or a combination thereof.

An Fc-silenced anti-RSV antibody comprising two heavy chain variable regions having an amino acid sequence as set forth in SEQ ID NO: 29 and two light chain variable regions having an amino acid sequence as set forth in SEQ ID NO: 30 was used as a negative control antibody (RSV control antibody).

A number of bispecific antibodies were selected for further screening, including bispecific antibodies comprising the following combination of heavy chain variable regions:

| LAG-3 VH | PD-L1 VH |
|---|---|
| SEQ ID NO: 1 | SEQ ID NO: 23 |
| SEQ ID NO: 3 | SEQ ID NO: 23 |
| SEQ ID NO: 5 | SEQ ID NO: 23 |
| SEQ ID NO: 6 | SEQ ID NO: 23 |
| SEQ ID NO: 15 | SEQ ID NO: 23 |
| SEQ ID NO: 16 | SEQ ID NO: 23 |

The bispecific antibodies were produced in Fc-silenced IgG1 format, and purified by protein A and gel filtration. Potential homodimer impurities were removed by CIEX. LAG-3 homodimer formation was mitigated by use of CH3 engineering technology to abridge potential impact in functional assays. Monospecific parental antibodies were included in the screening assays to compare the activity of bispecific antibodies to their parental IgG and to determine the sensitivity of the functional assays for PD-L1 and LAG-3 single targeting agents. The monospecific parental antibodies were produced and characterized in a similar fashion as the bispecific antibodies.

### Antigen Binding

Binding of the bispecific antibodies to LAG-3 and PD-L1 was analyzed by FACS using a 293FF cell line stably transfected with human and rhesus LAG-3 and a CHO-K1 cell line stably transfected with human and rhesus PD-L1.

The bispecific antibodies and parental antibodies were analyzed in an 8-step, 5-fold titration starting at 50 μg/mL. A goat-anti-human PE antibody (Invitrogen, H10104) was used as secondary antibody at a concentration of 3 μg/mL. In all plates containing cells expressing LAG-3, the relatlimab analog was used as a positive control, whereas the atezolizumab analog was used as a positive control for plates containing PD-L1 expressing cells. A 6-step, 5-fold titration starting at 50 μg/mL of the RSV control antibody was included on all plates as a negative control. Two wells were used for secondary antibody only and unstained cells, as additional controls. As a negative control, cells not expressing the target, 293FF and CHO-K1 cells, were included where the IgG's were only tested at the two highest concentrations (50 and 10 μg/mL).

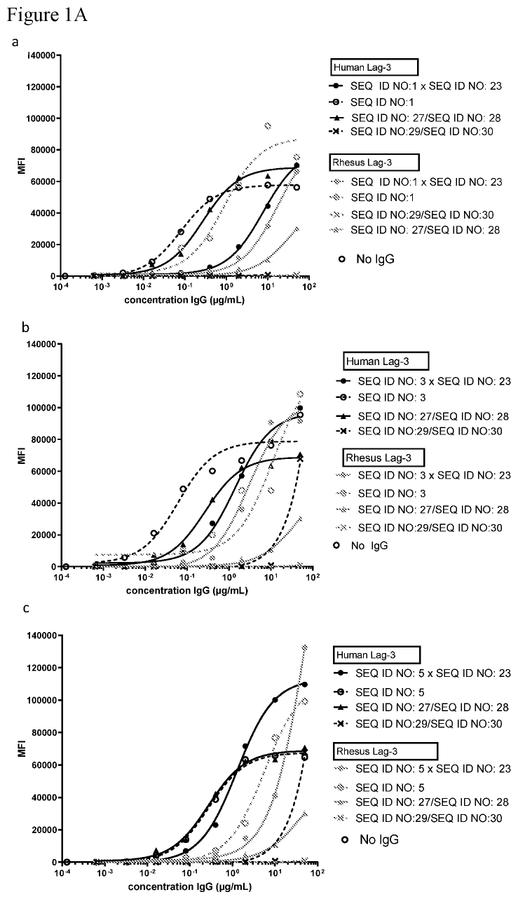

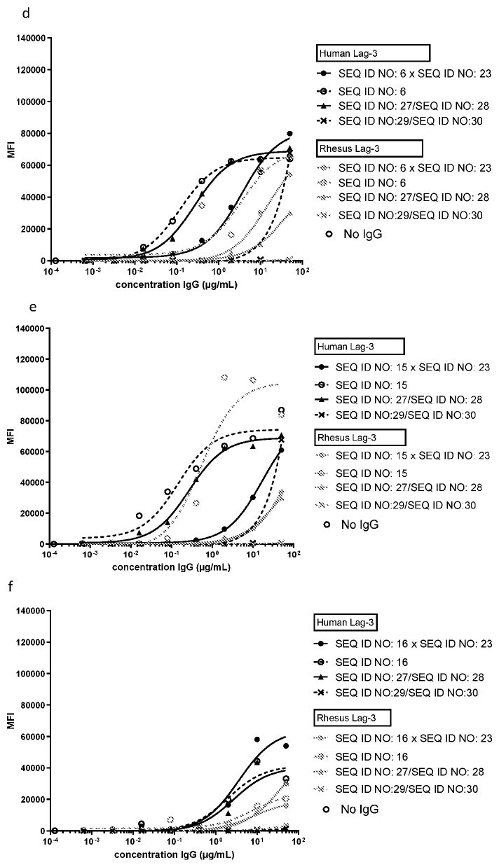

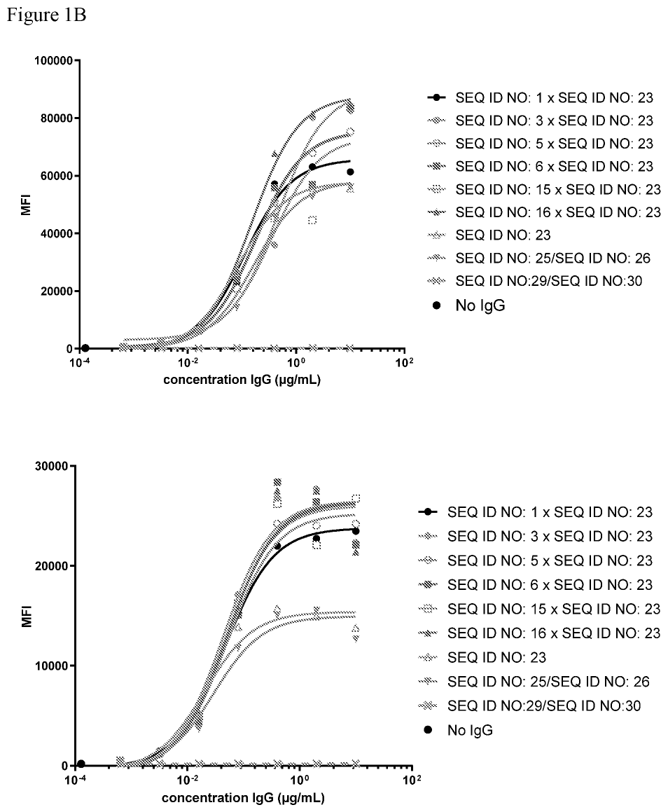

Results for LAG-3 binding are shown in FIG. 1A. EC50 values were calculated using GraphPad Prism software version 7.02 (nonlinear regression, 3-parameter dose-response curve). Each of the bispecific antibodies is plotted together with the positive control antibody, the negative control antibody and its parental LAG-3 binding antibody.

The positive control antibody relatlimab analog showed inconsistent binding to human LAG-3 across the three plates used in this assay: the degree of binding on the plate with the LAG-3 arm comprising SEQ ID NO: 16 was much lower than that observed on the other plates. EC50 values were higher for the bispecific antibodies (monovalent for LAG-3) than for the monospecific parental antibodies (bivalent for LAG-3). The LAG-3 arms comprising SEQ ID NO: 3 and 5 showed the highest degree of binding to human LAG-3. An overview of the EC50 values is shown in Table 3.

TABLE 3
EC50 values.

| | | | EC50 (nM) |
|---|---|---|---|
| Format | LAG-3 | PD-L1 | human LAG-3 |
| Monovalent for | SEQ ID NO: 1 | SEQ ID NO: 23 | 52.13 |
| LAG-3 | | | |
| Bivalent for | SEQ ID NO: 1 | NA | 0.54 |
| LAG-3 | | | |
| Monovalent for | SEQ ID NO: 3 | SEQ ID NO: 23 | 9.45 |
| LAG-3 | | | |
| Bivalent | SEQ ID NO: 3 | NA | 0.40 |
| Monovalent for | SEQ ID NO: 5 | SEQ ID NO: 23 | 8.64 |
| LAG-3 | | | |
| Bivalent for | SEQ ID NO: 5 | NA | 1.88 |
| LAG-3 | | | |
| Monovalent for | SEQ ID NO: 6 | SEQ ID NO: 23 | 23.92 |
| LAG-3 | | | |
| Bivalent for | SEQ ID NO: 6 | NA | 0.80 |
| LAG-3 | | | |
| Monovalent for | SEQ ID NO: 15 | SEQ ID NO: 23 | 113.16 |
| LAG-3 | | | |
| Bivalent for | SEQ ID NO: 15 | NA | 0.94 |
| LAG-3 | | | |
| Monovalent for | SEQ ID NO: 16 | SEQ ID NO: 23 | 24.05 |
| LAG-3 | | | |
| Bivalent for | SEQ ID NO: 16 | NA | 13.27 |
| LAG-3 | | | |

Results for PD-L1 binding are shown in FIG. 1B. EC50 values were calculated using GraphPad Prism software version 7.02 (nonlinear regression, 3-parameter dose-response curve). The bispecific antibodies are plotted together with the positive control antibody, the negative control antibody and the parental PD-L1 binding antibody.

All bispecific antibodies showed binding to CHO cells expressing huPD-L1. The maximal effect at the highest drug concentration (Emax) was higher for the bispecific antibody comprising SEQ ID NO: 23, monovalent for PD-L1, than for the bivalent monospecific parental antibody. A number of bispecific antibodies comprising the PD-L1 arm having SEQ ID NO: 23 showed better binding than, or comparable binding to, the atezolizumab analog. An overview of the EC50 values is shown in Table 4.

TABLE 4
EC50 values.

| | | | EC50 (nM) |
|---|---|---|---|
| Format | LAG-3 | PD-L1 | human PD-L1 |
| Monovalent for | SEQ ID NO: 1 | SEQ ID NO: 23 | 0.74 |
| PD-L1 | | | |
| Monovalent for | SEQ ID NO: 3 | SEQ ID NO: 23 | 3.02 |
| PD-L1 | | | |
| Monovalent for | SEQ ID NO: 5 | SEQ ID NO: 23 | 1.21 |
| PD-L1 | | | |
| Monovalent for | SEQ ID NO: 6 | SEQ ID NO: 23 | 1.21 |
| PD-L1 | | | |
| Monovalent for | SEQ ID NO: 15 | SEQ ID NO: 23 | 2.41 |
| PD-L1 | | | |
| Monovalent for | SEQ ID NO: 16 | SEQ ID NO: 23 | 1.01 |
| PD-L1 | | | |
| Bivalent for PD- | NA | SEQ ID NO: 23 | 0.54 |
| L1 | | | |
| Bivalent for PD- | NA | SEQ ID NO: 25 | 1.12 |
| L1 | | | |

### PD-1/TAG-3 Reporter Assay

A PD-1/LAG-3 reporter assay was performed using PD-1 and LAG-3 expressing Jurkat T cells as effector cells and PD-L1 expressing Raji cells as target cells. The PD-1 and LAG-3 effector cells were prepared and plated at 100,000 cells per well. Antibody solution was added followed by the Raji cells (25,000 per well). The T cells were activated by the addition of partially purified Staphylococcal enterotoxin D (ppSED, final concentration of 16.6 ng/mL). After 6 hours incubation at 37° C., luciferase reporter gene activity was determined by adding Bio-Glo reagent and measuring luminescence on an EnVision plate reader.

The bispecific IgG's were tested in a 6-step semi-log titration starting at 100 μg/mL (final concentration). IgG dilutions were prepared at 3× final concentration in assay medium. As a positive control, a 6-step semi-log titration of a combination of atezolizumab analog and relatlimab analog, with a highest concentration of 50 μg/mL+50 μg/mL, were included on each plate. Atezolizumab analog and relatlimab analog were also tested as single agents at 100 μg/mL starting concentration. As a negative control on each plate a 4-step semi-log titration of the RSV control antibody was used starting at 100 μg/mL. Two wells were left without IgG as a control for the basal level of activity.

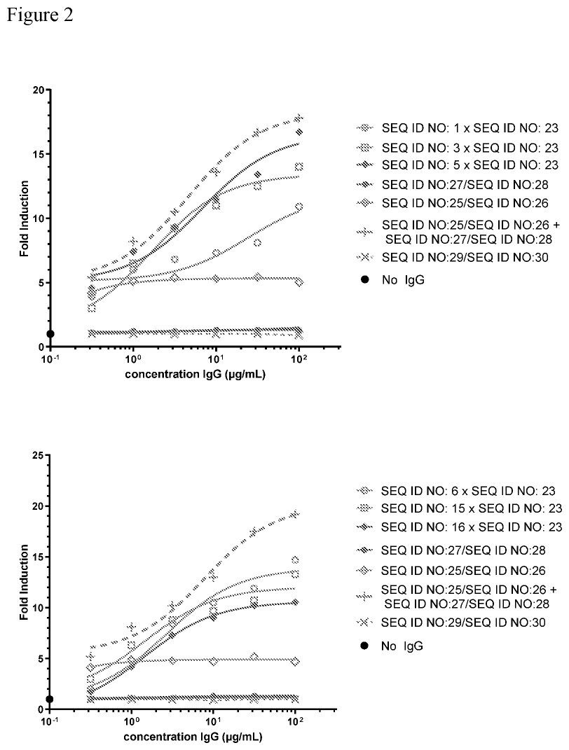

Results are shown in FIG. 2. The bispecific antibodies were compared with each other and with the atezolizumab analog, the relatlimab analog, as well as a combination of the relatlimab and atezolizumab analogs. The area under the curve (AUC) of the antibody response was determined using GraphPad Prism software version 7.02. The AUCs were expressed relative to the AUC of the positive control.

An overview of the AUC is shown in Table 5.

TABLE 5
AUC values.

| | | AUC compared |
|---|---|---|
| LAG-3 | PD-L1 | to control |
| SEQ ID NO: 5 | SEQ ID NO: 23 | 83.9 |
| SEQ ID NO: 3 | SEQ ID NO: 23 | 76.6 |
| SEQ ID NO: 6 | SEQ ID NO: 23 | 71.5 |
| SEQ ID NO: 15 | SEQ ID NO: 23 | 71.0 |
| SEQ ID NO: 16 | SEQ ID NO: 23 | 58.3 |
| SEQ ID NO: 1 | SEQ ID NO: 23 | 54.8 |

### SEB Assay

The bispecific antibodies and parental antibodies were analyzed in a SEB assay using cells from two donors: PBMC donor1 and PBMC donor2. First, PBMCs from these donors were shown to respond to the anti-LAG-3 reference antibody relatlimab analog and the anti-PD-L1 antibody atezolizumab analog (data not shown). In short, a dilution series of the IgG's were added to 2×10⁵ PBMC cells, followed by the addition of 2 μg/mL SEB (final concentration). IgG's were analyzed in a 6-step 7-fold dilution series starting at 50 μg/mL (final concentration). As a positive control, a 5-fold titration of a combination of the atezolizumab analog and relatlimab analog, starting at 25 μg/mL+25 μg/mL, was included on each plate. As a negative control on each plate a 4-step semi-log titration of the RSV-control antibody was used starting at 50 μg/mL. Two wells were left without IgG as a control for the basal level of activity. PBMCs were cultured for three days after which IL-2 and TNFα levels were measured in the supernatant by Luminex.

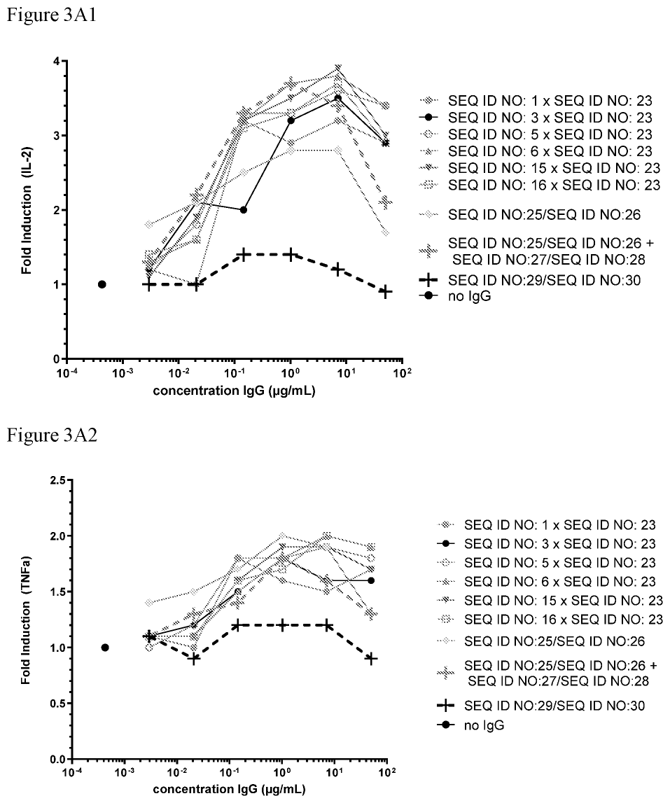

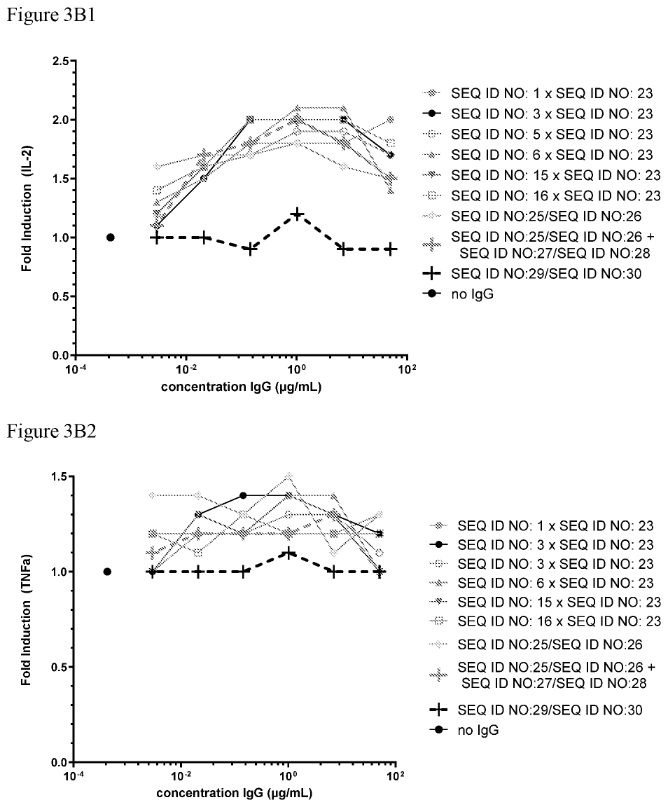

Results are shown in FIG. 3. The AUCs of the antibody response were determined using GraphPad Prism software version 7.02.

The standard curves for the cytokine measurements by Luminex were as expected, and the MFI values of all samples were in the range of the standard curves for IL-2 and TNFα (data not shown). The IL-2 data from the SEB assay are shown in FIG. 3A1 (donor1) and FIG. 3B1 (donor2). The data for TNFα is provided in FIG. 3A2 (donor1) and FIG. 3B2 (donor2).

In the first assay (PBMC donor1), the positive and negative controls were as expected for both the TL-2 and TNFα read-outs. The activity of the combination of atezolizumab and relatlimab analogs was higher than that for the atezolizumab analog alone for the IL-2 read-out. For both IL-2 and TNFα read-outs, the LAG-3×PD-L1 bispecific antibodies were at least as potent as the combination of atezolizumab and relatlimab analogs.

In the second assay (PBMC donor2), the positive and negative controls were as expected for IL-2. However, the degree of TNFα induction for the positive control was rather low. Again, the activity of the combination of atezolizumab and relatlimab analogs was higher than that for the atezolizumab analog alone for the IL-2 read-out. For both read-outs, the LAG-3×PD-L1 bispecific antibodies were at least as potent as the combination of atezolizumab and relatlimab analogs. The activity of the LAG-3×PD-L1 bispecific antibodies correlated well between the two donors. The bispecific antibodies with the LAG-3 arms having an amino acid sequence as set forth in SEQ ID NO: 5, SEQ ID NO: 15 and SEQ ID NO: 16 showed similar activity to one another and were at least as potent as the combination of atezolizumab and relatlimab analogs.

### Example 3—Binding Affinity of Bispecific Antibodies Comprising Further LAG-3 and PD-L1 Heavy Chain Variable Regions

Binding affinity of bispecific antibodies comprising a LAG-3 binding domain with a heavy chain variable region having an amino acid sequence as set forth in SEQ ID NO: 5 and a PD-L1 binding domain with a heavy chain variable region having an amino acid sequence as set forth in SEQ ID NO: 20, and a bispecific antibody comprising a LAG-3 binding domain with a heavy chain variable region having an amino acid sequence as set forth in SEQ ID NO: 17 and a PD-L1 binding domain with a heavy chain variable region having an amino acid sequence as set forth in SEQ ID NO: 24, was determined by SPR technology using a BIAcore T200.

Bispecific antibody binding to four recombinant protein antigens sourced from Sino Biological (human LAG-3-His, Cat nr. 16498-H08H; cynomolgus monkey LAG-3-His, Cat nr. 90841-C08H; human PD-L1-His, Cat nr. 10084-H08H; cynomolgus monkey PD-L1-His, Cat nr. 90251-C08H) was determined in separate experiments. In these assays, anti-huIgG Fcγ (JIR; Cat nr. 109-005-098) is covalently coupled to the surface of a CM5 sensor chip using free amine chemistry: the capturing antibody is diluted in a kAc buffer to 40 μg/mL and coupled to a surface that is activated with NHS/EDC (according to the manufacturer's recommendations). Next, bispecific antibodies are injected at concentrations up to 20 nM at 30 μL/min for 2 minutes in the flow cell. Subsequently, antigen (0.6-20 nM) in 0.01 M HEPES, 0.5 M NaCl, 0.003 M EDTA and 0.05% v/v Surfactant P20 buffer is flowed over the surface of the CM5 sensor chip at 30 μL/min for 2 minutes. Sensorgrams of the association and dissociation phases for the different antigens are thus obtained. Using the BIA evaluation software and curve-fitting employing a 1:1 interaction model (for monovalent interaction), the affinities of the individual Fab arms are determined.

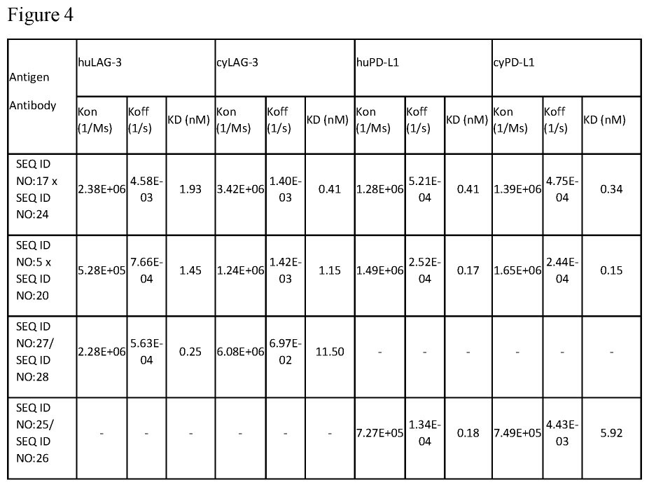

Results are shown in FIG. 4. A bispecific antibody comprising a LAG-3 binding domain with a heavy chain variable region having an amino acid sequence as set forth in SEQ ID NO: 5 and a PD-L1 binding domain with a heavy chain variable region having an amino acid sequence as set forth in SEQ ID NO: 20, and a bispecific antibody comprising a LAG-3 binding domain with a heavy chain variable region having an amino acid sequence as set forth in SEQ ID NO: 17 and a PD-L1 binding domain with a heavy chain variable region having an amino acid sequence as set forth in SEQ ID NO: 24, have a lower binding affinity for human LAG-3 and a higher binding affinity for cynomolgus LAG-3 as compared to the relatlimab analog. The bispecific antibodies have a similar binding affinity for human PD-L1 and a higher binding affinity for cynomolgus PD-L1 as compared to the atezolizumab analog.

Both bispecific antibodies bind to human LAG-3 and human PD-L1 simultaneously (data not shown).

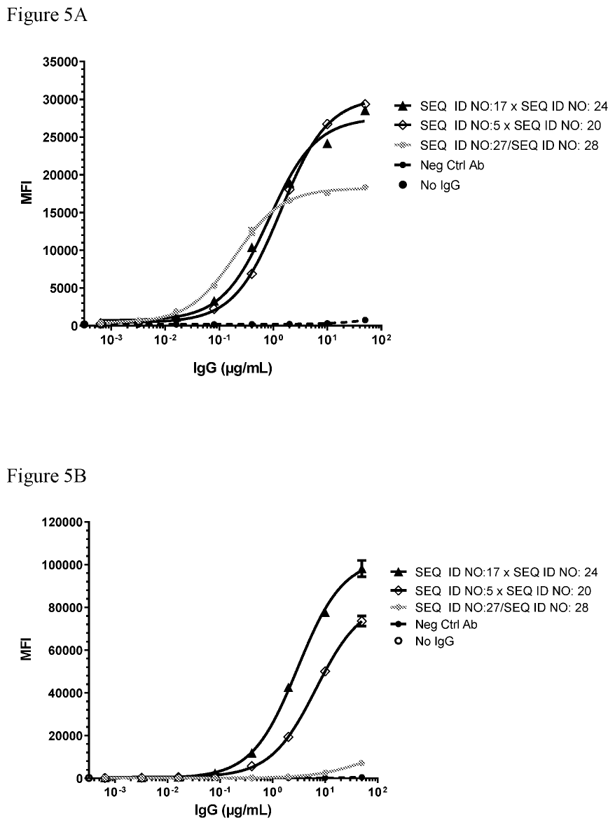

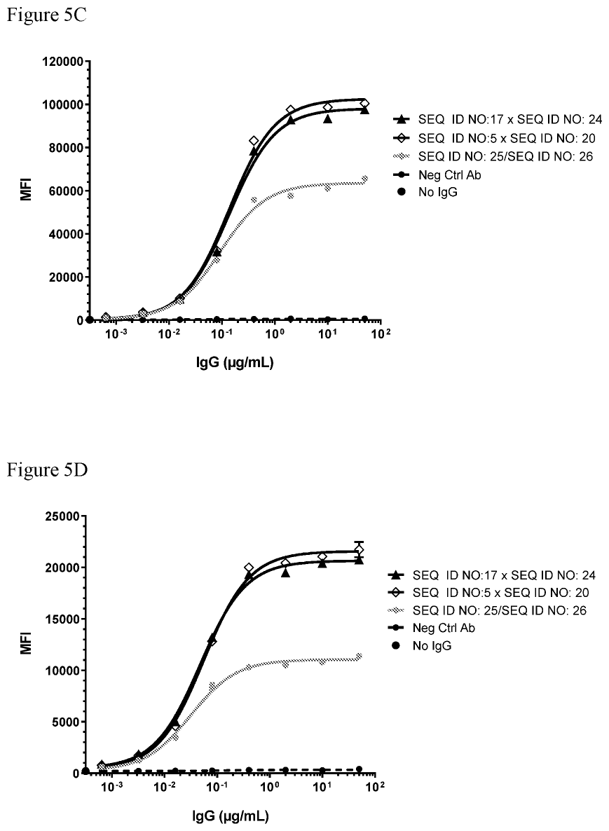

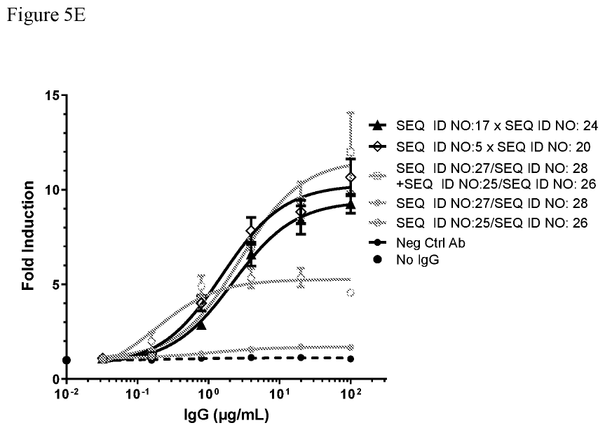

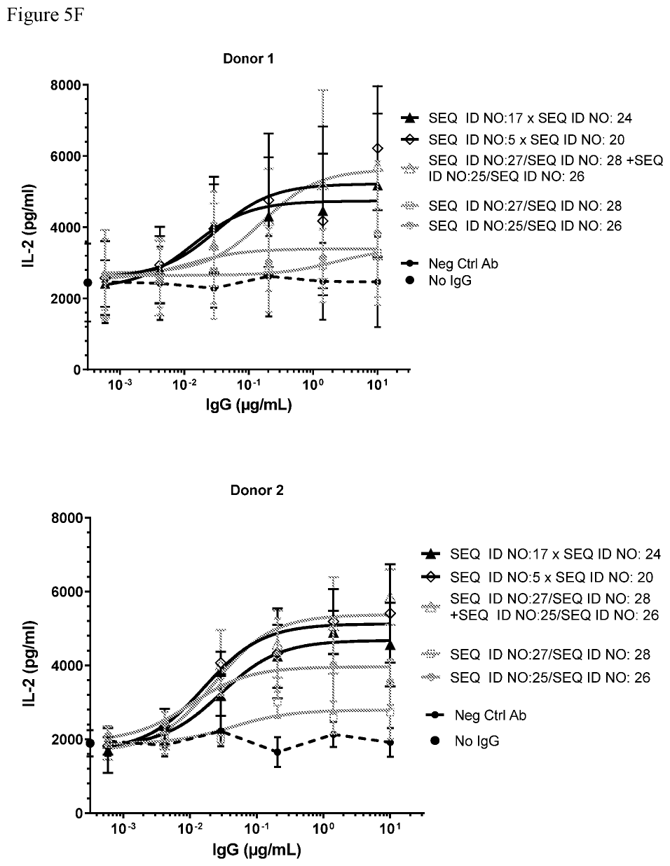

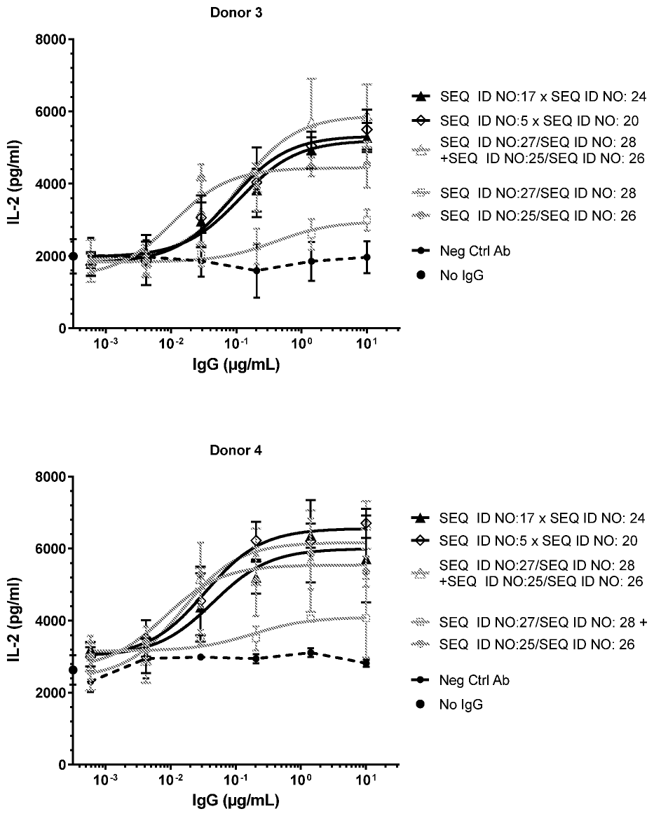

These antibodies were screened in binding, PD-1/LAG-3 reporter, and SEB assays as described in Example 2 to confirm their properties. Results are shown in FIG. 5. Both bispecific antibodies bind human and rhesus LAG-3 (FIGS. 5A and B) and human and rhesus PD-L1 (FIGS. 5C and D). Both bispecific antibodies show comparable activity to a combination of relatlimab and atezolizumab analogs in the reporter assay (FIG. 5E) and SEB assay (FIG. 5F). EC50 values are provided in Tables 6 and 7.

TABLE 6
EC50 values from the PD-1/LAG-3 reporter assay.

| LAG-3 | PD-L1 | EC50 (nM) |
|---|---|---|
| SEQ ID NO: 17 | SEQ ID NO: 24 | 14.80 |
| SEQ ID NO: 5 | SEQ ID: NO 20 | 10.43 |

TABLE 7
EC50 values from the SEB assay.

| LAG-3 | PD-L1 | EC50 ± SD (nM) |
|---|---|---|
| SEQ ID NO: 17 | SEQ ID NO: 24 | 0.34 ± 0.29 |
| SEQ ID NO: 5 | SEQ ID: NO 20 | 0.27 ± 0.19 |

### Example 4—In Vivo Study

Anti-tumor efficacy of bispecific antibodies in comparison with relatlimab and atezolizumab analogs were evaluated in a human stem cell humanized NSG mouse model bearing orthotopic MDA-MB-231 tumors.

Female hu-CD34 NSG™ mice (JAX stock #705557) that have been engrafted with human CD34+ cells and have >25% human CD45+ cells in the peripheral blood 14 weeks post engraftment but not later than 24 weeks were used. Cohorts of hu-CD34 NSG™ mice engrafted with CD34⁺ cells from three independent donors were used. 120 HSC-NSG mice (72+overage) were engrafted with 5×10⁶ MDA-MB-231 human breast adenocarcinoma cells, re-suspended in PBS with matrigel in a 1:1 ratio, implanted into the mammary fat pad. HSC-NSG mice are immuno-deficient NOD.Cg-PrkdC^{scid} 1l2rg^{tm1Wj1}/SzJ (NSG™) mice engrafted with human CD34⁺ hematopoietic stem cells (HSC) from cord blood, which undergo multilineage differentiation into all major immune cell types.

72 mice were enrolled 15 days post tumor-inoculation, when tumor volume (TV) was between 50-150 mm³. Tumor volume for enrollment was measured the previous day (Day −1). Dosing was initiated on the day of enrollment, designated Day 0 of treatment. Each treatment arm (Table 1) had mice from 3 HSC donors, 4 mice per donor (N=12). Tumor size and body weight were measured twice weekly.

TABLE 8
Study Design.

| | | | Dose | | |
|---|---|---|---|---|---|
| | | | level | Dose | Dosing |
| Group | N | Treatment | (mg/kg) | Route | Frequency |
| 1 | 12 (4 × 3 | Vehicle- PBS | — | i.p | q5dX9 |
| | donors) | | | | |
| 2 | 12 (4 × 3 | relatlimab* | 5 | i.p | q5dX9 |
| | donors) | | | | |
| 3 | 12 (4 × 3 | atezolizumab* | 5 | i.p | q5dX9 |
| | donors) | | | | |
| 4 | 12 (4 × 3 | relatlimab* | 5 | i.p | q5dX9 |
| | donors) | atezolizumab* | 5 | i.p | q5dX9 |
| 5 | 12 (4 × 3 | SEQ ID NO: 5 × | 10 | i.p | q5dX9 |
| | donors) | SEQ ID NO: 20 | | | |
| 6 | 12 (4 × 3 | SEQ ID NO: 17 × | 10 | i.p | q5dX9 |
| | donors) | SEQ ID NO: 24 | | | |

*Analog antibody

Terminal tumors were isolated at 24 hours post final dose for flow cytometry analysis of CD4⁺ and CD8⁺ T cell and regulatory T cell (Treg) populations in MDA-MB-231 tumor samples harvested on the day of termination.

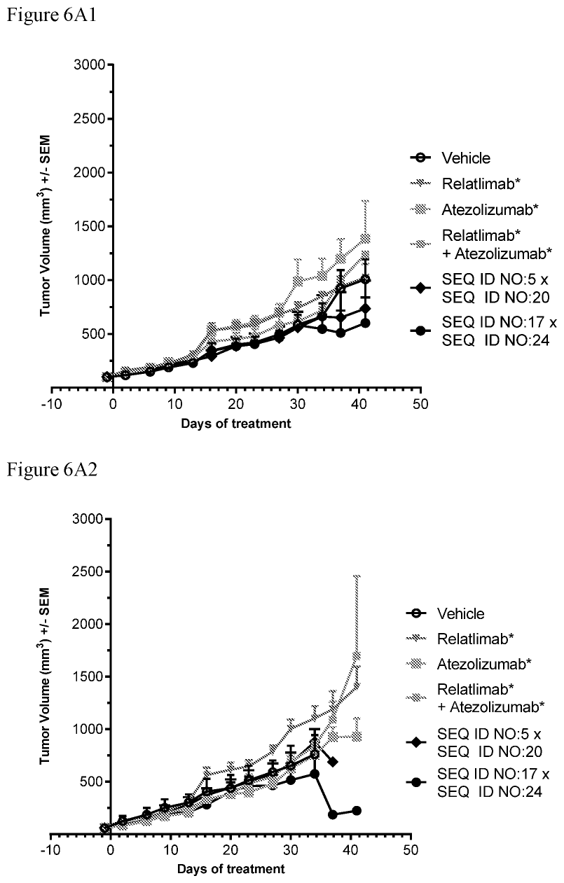

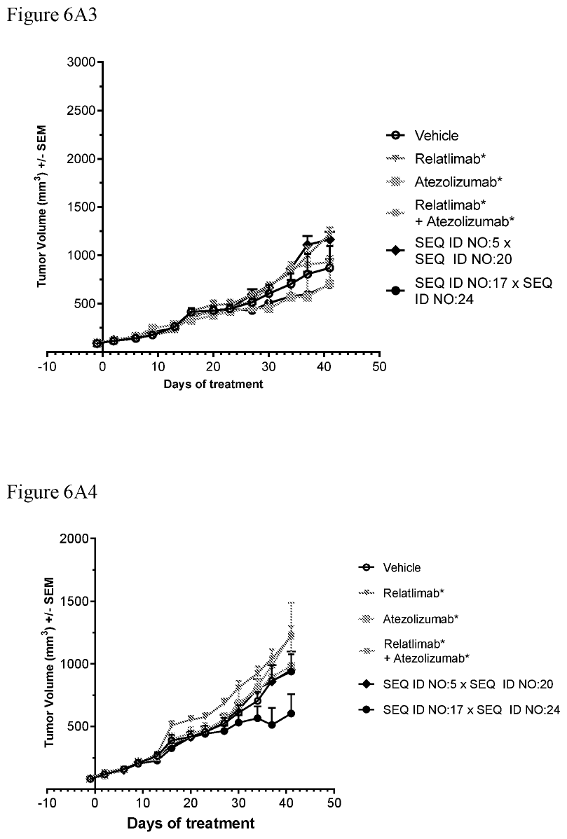

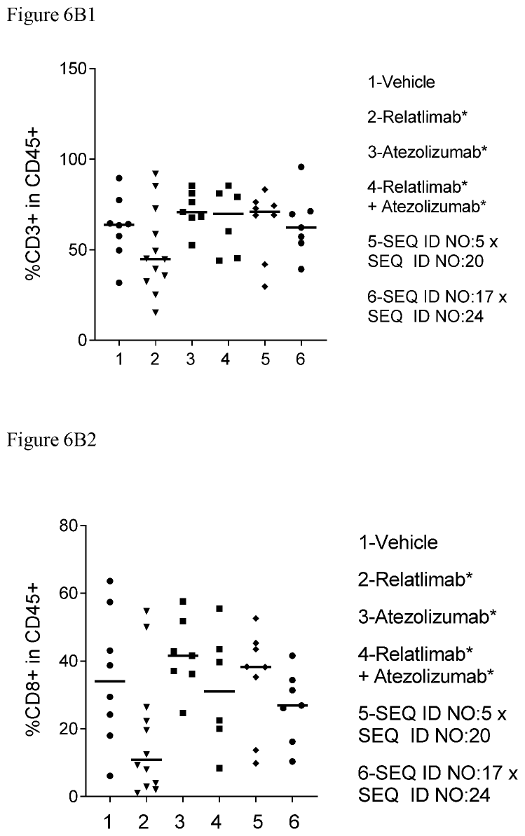

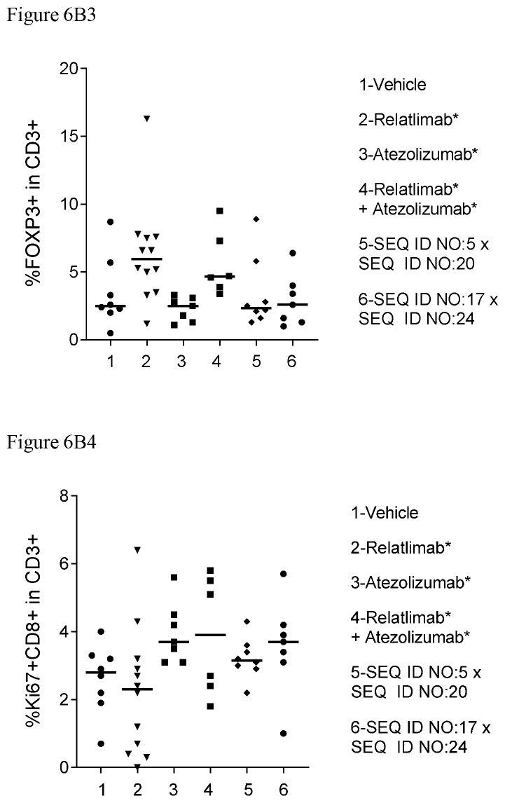

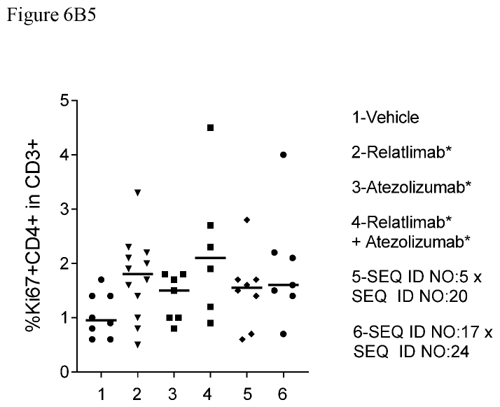

Results are shown in FIG. 6. Single and combination treatment of reference anti-LAG-3 antibody relatlimab analog and anti-PD-L1 antibody atezolizumab analog were not effective. Decreased tumor size in mice treated with bispecific antibody comprising a heavy chain variable region having an amino acid sequence as set forth in SEQ ID NO: 17, a heavy chain variable region having an amino acid sequence as set forth in SEQ ID NO: 24, and two light chain variable regions having an amino acid sequence as set forth in SEQ ID NO: 30, is significant when data from all donors are combined (FIG. 6A).

Tumor growth profile is presented in Table 9.

TABLE 9
Tumor growth profile.

| | | | | | SEQ ID | SEQ ID |
|---|---|---|---|---|---|---|
| | | | | relatlimab* | NO: 5 × | NO: 17 × |
| | | | | 5 mg/kg + | SEQ ID | SEQ ID |
| | | relatlimab* | atezolizumab* | atezolizumab* | NO: 20 | NO: 24 |
| | Vehicle | 5 mg/kg | 5 mg/kg | 5 mg/kg | 10 mg/kg | 10 mg/kg |
| PROGRESSIVE | 11 | 10 | 8 | 10 | 9 | 7 |
| STABLE | 1 | 2 | 2 | 2 | 3 | 3 |
| REGRESSING | 0 | 0 | 1 | 0 | 0 | 1 |
| Complete | 0 | 0 | 1 | 0 | 0 | 1 |
| regression | | | | | | |
| (TV = 0) | | | | | | |
| Total | 12 | 12 | 12 | 12 | 12 | 12 |
| % progressive | 92 | 83 | 67 | 83 | 75 | 58 |

*Analog antibody

FIG. 6B4 shows that PD-L1 and LAG-3×PD-L1 treatment increases the percentage of proliferating CD8⁺. LAG-3, PD-L1, and LAG-3×PD-L1 treatment increases the percentage of proliferating CD4⁺ T cells (FIG. 6B5). FIG. 6B3 shows that treatment with the bispecific antibodies results in a lower percentage of Tregs than treatment with a combination of relatlimab and atezolizumab analogs.

### Example 5: Screening of Further LAG-3×PD-L1 Bispecific Antibodies

The bispecific antibodies described in Table 10 were screened essentially as described in Example 2.

TABLE 10
Overview of further LAG-3xPD-L1 bispecific antibodies.

| | Bispecific antibody | | | | | | | |
|---|---|---|---|---|---|---|---|---|
| | LAG-3 binding domain | | | | PD-L1 binding domain | | | |
| | VH | CH1 | CH2 | CH3 | VH | CH1 | CH2 | CH3 |
| SEQ ID NO: 5 × | SEQ ID | SEQ ID | SEQ ID | SEQ ID | SEQ ID | SEQ ID | SEQ ID | SEQ ID |
| SEQ ID NO: 20 | NO: 5 | NO: 42 | NO: 45 | NO: 50 | NO :20 | NO: 42 | NO: 45 | NO: 51 |
| SEQ ID NO: 52 × | SEQ ID | SEQ ID | SEQ ID | SEQ ID | SEQ ID | SEQ ID | SEQ ID | SEQ ID |
| SEQ ID NO: 20 | NO: 52 | NO: 42 | NO: 45 | NO: 50 | NO :20 | NO: 42 | NO: 45 | NO: 51 |
| SEQ ID NO: 17 × | SEQ ID | SEQ ID | SEQ ID | SEQ ID | SEQ ID | SEQ ID | SEQ ID | SEQ ID |
| SEQ ID NO: 24 | NO: 17 | NO: 42 | NO: 45 | NO: 51 | NO :24 | NO: 42 | NO: 45 | NO: 50 |
| SEQ ID NO: 53 × | SEQ ID | SEQ ID | SEQ ID | SEQ ID | SEQ ID | SEQ ID | SEQ ID | SEQ ID |
| SEQ ID NO: 24 | NO: 53 | NO: 42 | NO: 45 | NO: 51 | NO :24 | NO: 42 | NO: 45 | NO: 50 |
| SEQ ID NO: 54 × | SEQ ID | SEQ ID | SEQ ID | SEQ ID | SEQ ID | SEQ ID | SEQ ID | SEQ ID |
| SEQ ID NO: 24 | NO: 54 | NO: 42 | NO: 45 | NO: 51 | NO :24 | NO: 42 | NO: 45 | NO: 50 |
| SEQ ID NO: 55 × | SEQ ID | SEQ ID | SEQ ID | SEQ ID | SEQ ID | SEQ ID | SEQ ID | SEQ ID |
| SEQ ID NO: 24 | NO: 55 | NO: 42 | NO: 45 | NO: 51 | NO :24 | NO: 42 | NO: 45 | NO: 50 |
| SEQ ID NO: 56 × | SEQ ID | SEQ ID | SEQ ID | SEQ ID | SEQ ID | SEQ ID | SEQ ID | SEQ ID |
| SEQ ID NO: 24 | NO: 56 | NO: 42 | NO: 45 | NO: 51 | NO :24 | NO: 42 | NO: 45 | NO: 50 |
| SEQ ID NO: 57 × | SEQ ID | SEQ ID | SEQ ID | SEQ ID | SEQ ID | SEQ ID | SEQ ID | SEQ ID |
| SEQ ID NO: 24 | NO: 57 | NO: 42 | NO: 45 | NO: 51 | NO :24 | NO: 42 | NO: 45 | NO: 50 |
| SEQ ID NO: 58 × | SEQ ID | SEQ ID | SEQ ID | SEQ ID | SEQ ID | SEQ ID | SEQ ID | SEQ ID |
| SEQ ID NO: 24 | NO: 58 | NO: 42 | NO: 45 | NO: 51 | NO :24 | NO: 42 | NO: 45 | NO: 50 |
| SEQ ID NO: 59 × | SEQ ID | SEQ ID | SEQ ID | SEQ ID | SEQ ID | SEQ ID | SEQ ID | SEQ ID |
| SEQ ID NO: 24 | NO: 59 | NO: 42 | NO: 45 | NO: 51 | NO :24 | NO: 42 | NO: 45 | NO: 50 |

### Antigen Binding

Binding of the bispecific antibodies to LAG-3 and PD-L1 was analyzed by FACS using a 293FF cell line stably transfected with human and rhesus LAG-3 and a CHO-K1 cell line stably transfected with human and rhesus PD-L1, as in Example 2.

The bispecific antibodies were analyzed in an 8-step, 4-fold titration starting at 100 μg/mL on 293FF-huLAG-3 and 293FF-reLAG-3 cells and 25 μg/mL on CHO-K1-huPD-L1 cells. A goat-anti-human PE antibody (Invitrogen, H10104) was used as secondary antibody at a concentration of 3 μg/mL. RSV control antibody was included on all plates as a negative control, in an 8-step, 4-fold dilution, starting at 100 μg/mL on 293FF-huLAG-3 and 293FF-reLAG-3 cells, and starting at 25 μg/mL on CHO-K1-huPD-L1 cells. Two wells were used for secondary antibody only and unstained cells, as additional controls. As a negative control, cells not expressing the target, 293FF and CHO-K1 cells, were included where the IgG's were tested at the three highest concentrations: 100, 25 and 6.3 μg/mL on 293FF cells and 25, 6.3 and 1.6 μg/mL on CHO-K1 cells.

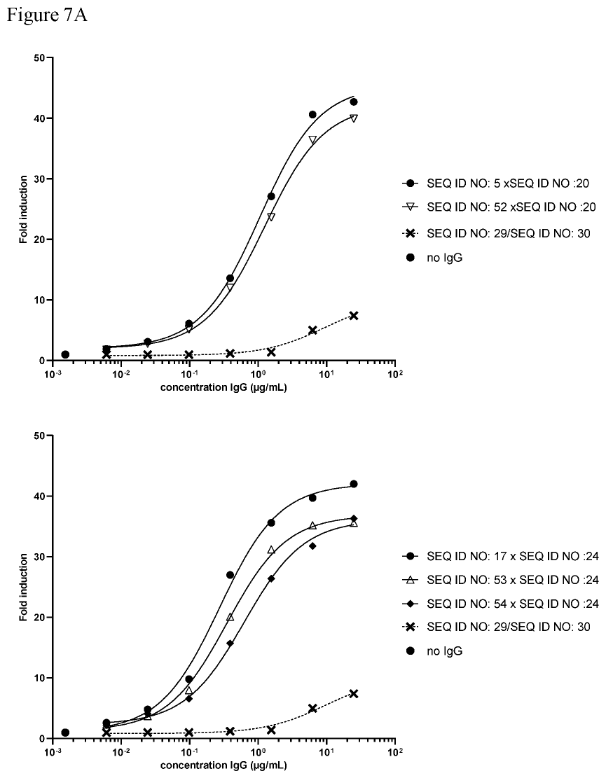

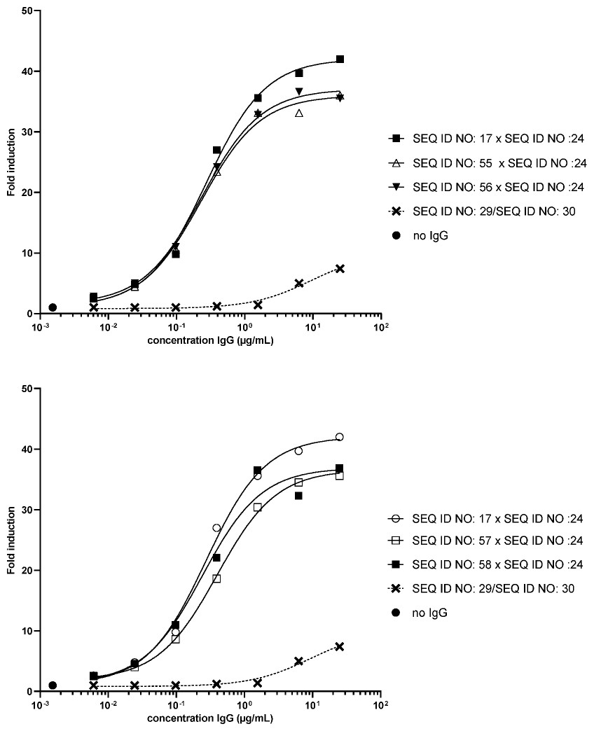

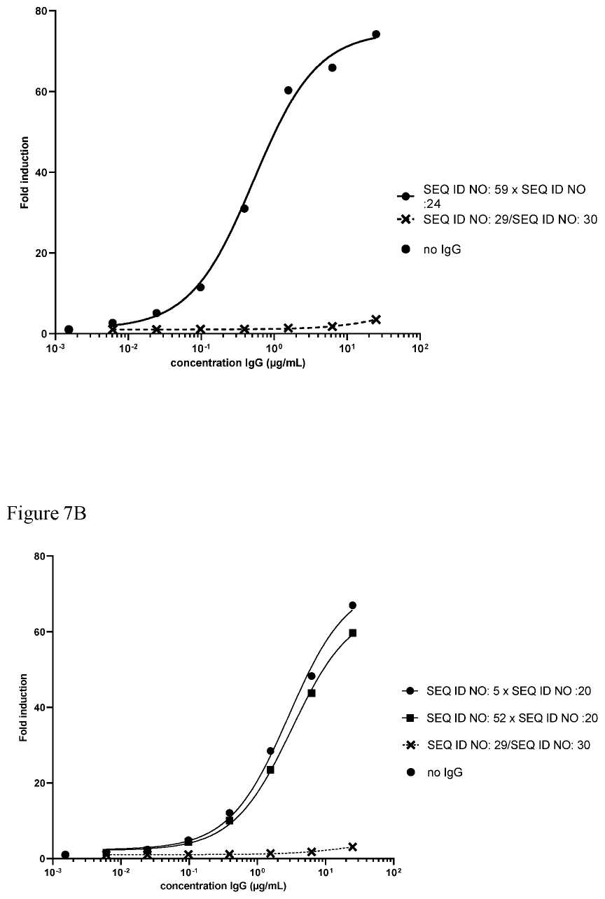

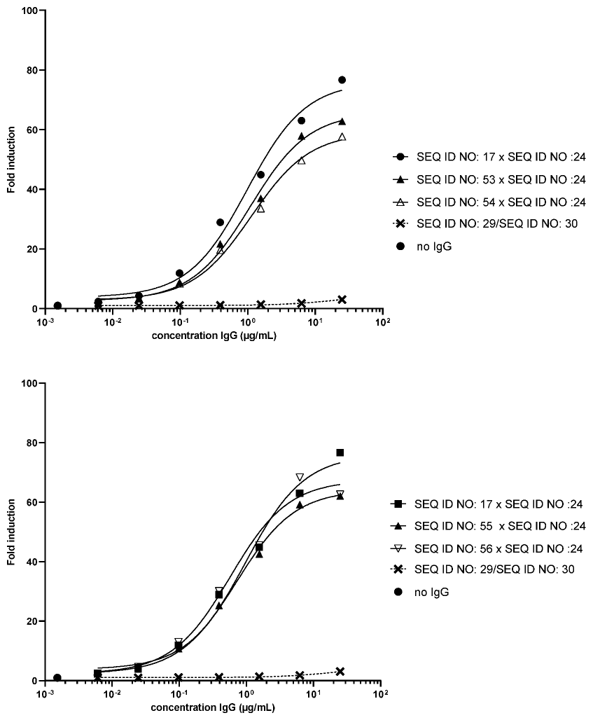

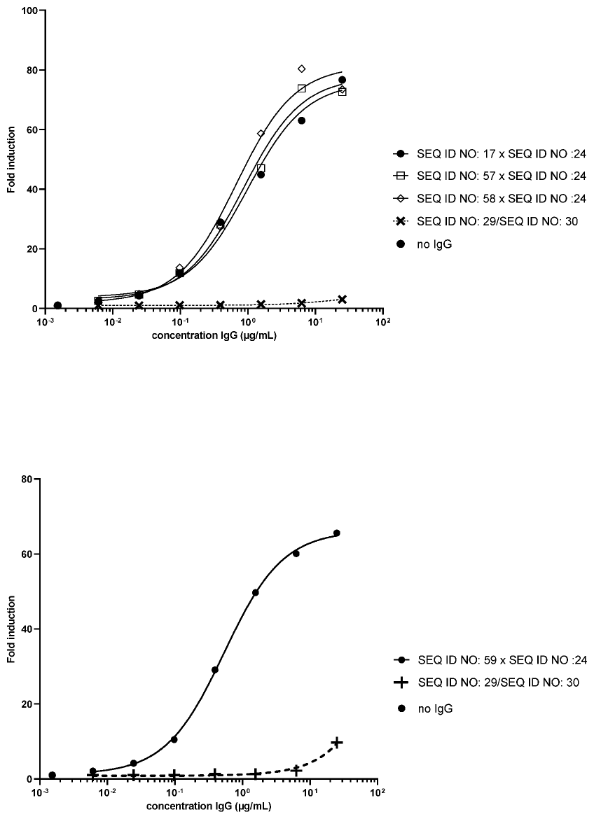

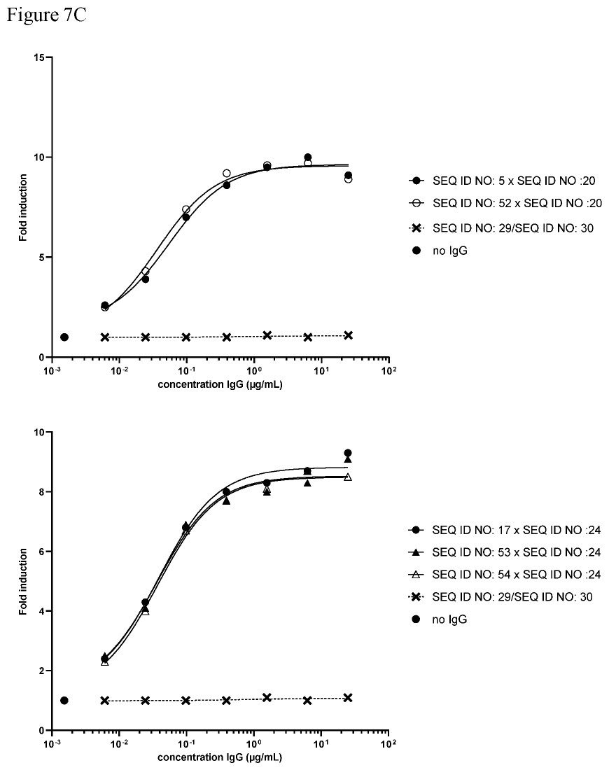

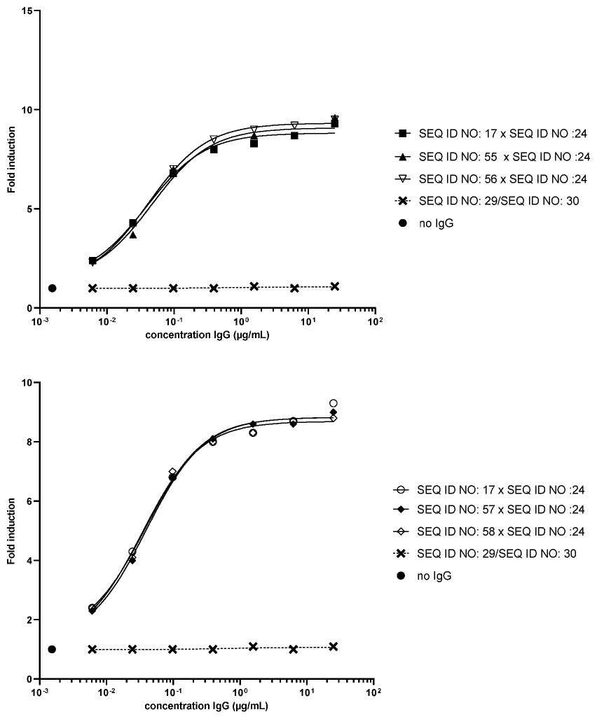

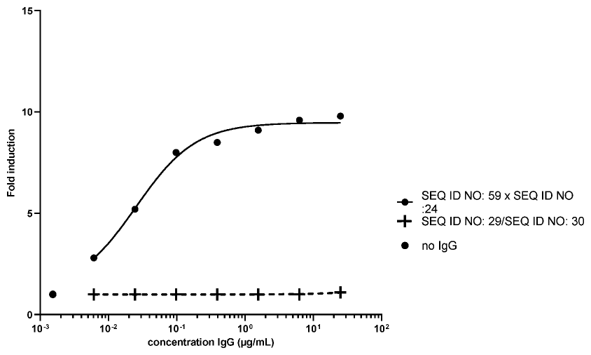

Results for human and rhesus LAG-3 binding are shown in FIGS. 7A and 7B. Results for PD-L1 binding are shown in FIG. 7C. Fold induction was obtained by normalizing mean MFI values with respect to that of wells without antibody and plotted as a function of the log of antibody concentration. EC50 values were calculated using GraphPad Prism software version 7.02 (nonlinear regression, 3-parameter dose-response curve). Each of the bispecific antibodies is plotted together with the negative control antibody.

All bispecific antibodies showed binding to human and rhesus LAG-3. All bispecific antibodies showed binding to CHO cells expressing huPD-L1. The EC50 values are provided in Table 11.

TABLE 11
EC50 and AUC values of the binding of the bispecific
antibodies to human and rhesus LAG-3 and human PD-L1.

| | | 293FF - Human | 293FF - Rhesus | CHO-K1 Human |
|---|---|---|---|---|
| | | LAG-3 | LAG-3 | PD-L1 |
| LAG-3 | PD-L1 | EC50 (μg/mL) | EC50 (μg/mL) | EC50 (μg/mL) |
| SEQ ID NO: 5 | SEQ ID NO: 20 | 1.06 | 2.88 | 0.0135 |
| SEQ ID NO: 52 | SEQ ID NO: 20 | 1.25 | 3.22 | 0.009 |
| SEQ ID NO: 17 | SEQ ID NO: 24 | 0.27 | 0.98 | 0.0097 |
| SEQ ID NO: 53 | SEQ ID NO: 24 | 0.35 | 1.09 | 0.0091 |
| SEQ ID NO: 54 | SEQ ID NO: 24 | 0.63 | 1.13 | 0.0093 |
| SEQ ID NO: 55 | SEQ ID NO: 24 | 0.23 | 0.72 | 0.0117 |
| SEQ ID NO: 56 | SEQ ID NO: 24 | 0.24 | 0.57 | 0.0103 |
| SEQ ID NO: 57 | SEQ ID NO: 24 | 0.4 | 0.86 | 0.0098 |
| SEQ ID NO: 58 | SEQ ID NO: 24 | 0.24 | 0.67 | 0.0086 |
| SEQ ID NO: 59 | SEQ ID NO: 24 | 0.52 | 0.54 | 0.0064 |

### PD-1/LAG-3 Reporter Assay

A PD-1/LAG-3 reporter assay was performed essentially as described in Example 2. The Staphylococcal enterotoxin D (ppSED) was validated to be used at a final concentration of 150 ng/mL.

The bispecific IgG's were tested in a 6-step semi-log titration starting at 100 μg/mL (final concentration). IgG dilutions were prepared at 3× final concentration in assay medium. As a positive control, a 6-step semi-log titration of a combination of atezolizumab analog and relatlimab analog, with a highest concentration of 50 μg/mL+50 μg/mL, was included on each plate. As a negative control on each plate a 4-step semi-log titration of the RSV control antibody was used starting at 100 μg/mL. Two wells were left without IgG as a control for the basal level of activity.

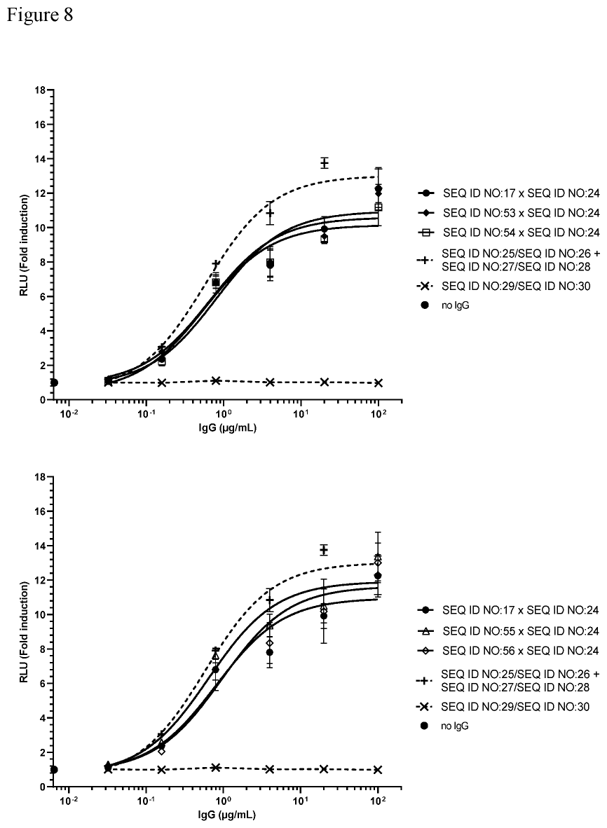

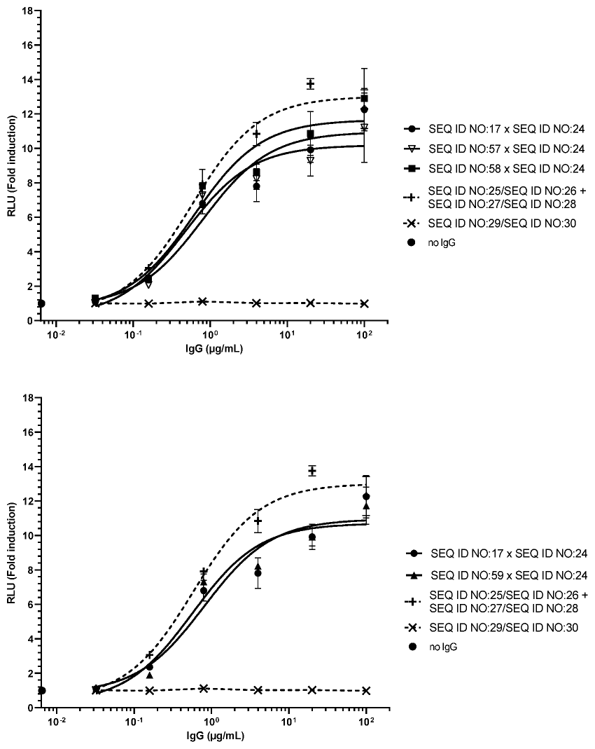

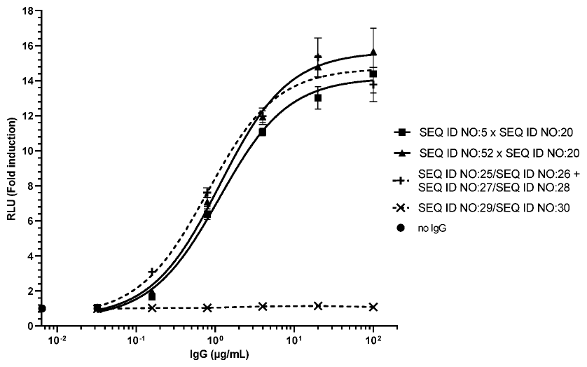

Results are shown in FIG. 8. All LAG-3×PD-L1 bispecific antibodies show activity as compared to a combination of relatlimab and atezolizumab analogs. EC50 and area under the curve (AUC) of the antibody response was determined using GraphPad Prism software version 7.02. The AUCs were expressed relative to the AUC of the combination of relatlimab and atezolizumab analogs.

TABLE 12
EC50 and AUC values of the bispecific antibodies
as determined in a PD-1/LAG-3 reporter assay.

| | | | AUC (% of |
|---|---|---|---|
| LAG-3 | PD-L1 | EC50 μg/mL) | control) |
| SEQ ID NO: 52 | SEQ ID NO: 20 | 1.154 | 96.93 |
| SEQ ID NO: 5 | SEQ ID NO: 20 | 1.132 | 86.25 |
| SEQ ID NO: 59 | SEQ ID NO: 24 | 0.5736 | 77.11 |
| SEQ ID NO: 58 | SEQ ID NO: 24 | 0.6303 | 85.47 |
| SEQ ID NO: 57 | SEQ ID NO: 24 | 0.481 | 75.11 |
| SEQ ID NO: 56 | SEQ ID NO: 24 | 0.9666 | 79.11 |
| SEQ ID NO: 55 | SEQ ID NO: 24 | 0.6529 | 86.97 |
| SEQ ID NO: 54 | SEQ ID NO: 24 | 0.5695 | 73.64 |
| SEQ ID NO: 53 | SEQ ID NO: 24 | 0.6613 | 77.11 |
| SEQ ID NO: 17 | SEQ ID NO: 24 | 0.815 | 76.53 |

SEQUENCES
SEQ ID NO: 1
QVQLQESGPGLVRPSETLSLTCTVSGGSISSYSWSWIRQPPGKGLEWIGY
IDYSGSTNYNPSLKSRVTISVDTSKTQFSLKLSSVSAADTAVYYCAKDLL
YKWNYVEGFDIWGQGTTVTVSS
SEQ ID NO: 2
QVQLQESGPGLVKPSETLSLTCTVSGGSISSYSWSWIRQPPGKGLEWIGY
IDYSGTTNENPSLKSRVTISVDTSKTQFSLKLSSVSAADTAVYYCAKDLL
YKWNYVEGFDIWGQGTMVTVSS
SEQ ID NO: 3
EVQLVESGGGVVQPGRSLRLSCAASGFTFSSYDTHWVRQAPGKGLEWVAV
ISYDGSNKYYADSVKGRFTISRDNSKNTLYLQMNSLRAEDTAMYYCARER
GWDVEDIWGQGTLVTVSS
SEQ ID NO: 4
QVQLVQSGAEVKKPGSSVKVSCKASGGTFSKYVVSWVRQAPGQGFDWMGG
IIPMFGTANYAQMFQGRVTITADKSTSTVNMELSSLRSEDTAVYYCVRDK
AVAGLYYFDSWGQGTLVTVSS
SEQ ID NO: 5
QVQLQESGPGLVKPSETLSLTCTVSDDSISDYYWSWIRQPPGKGLEWIGY
IYYSGNTKYNPSLKNRVTISVDTSKSQFSLKLTSVTAADTAVYYCARIPL
TGEFDYWAQGTLVTVSS
SEQ ID NO: 6
EVQLVESGGGVVQPGRSLRLSCAASGFTFSSYGMHWVRQAPGKGLEWVAV
ISYHGSDKYYADSVKGRFTISRDNSKNTLYLQMNSLRAEDTAVYYCARDG
DNWDVFDIWGQGTLVTVSS
SEQ ID NO: 7
QVQLVQSGAEVKKPGSSVKVSCKASGDTFSTYAINWIRQAPGQGLEWMGG
IIPIFGTAYYAQEFQDRVTITADKSTSTGYMEMSSLISEDTAVYYCARER
ELGALYAFDIWGQGTMVTVSS
SEQ ID NO: 8
EVQLVQSGAEVKKPGSSVKVSCKASGGTFSSHAISWVRQVPGQGLEWMGG
IIPLEDTAKNAQKFQGRVTITADKSTSTAYMELSSLRSEDTAVYYCARDR
ETGTLYYFDYWGQGTLVTVSS
SEQ ID NO: 9
EVQLVQSGSELKKPGASVKVSCKASGYTFTTNALNWVRQAPGQGLEWMGW
INTHTGNPTYAQGFIGRFVFSLDTSVSTAYLQIRSLKAEDTAVYYCAREP
NWGVYFDYWGQGTLVTVSS
SEQ ID NO: 10
QVQLVQSGAEVKRPGASVKVSCKVSGYTLTELSMHWVRQAPGKGLEWMGG
SDPEHGETVDAQKFQGRVTMTEDTSTDTAYMELSSLRSEDTAVYYCTTGG
TYYYGSGSYYTLDFWGQGTLVTVSS
SEQ ID NO: 11
QVQLVQSGAEVKKPGSSVKVSCKASGGTFSNFAFSWVRQAPGQGLEWMGG
IIPMEDTAKYAQKFQGRVTIIADKSTNTAYMDLNSLRSEDTAVYYCVRDR
AIGTLYYFDYWGQGTLVTVSS
SEQ ID NO: 12
QVQLVQSGAEVRKPGSSVMVSCKASGGTFNTYAINWVRQAPGQGLEWMGG
IIPIFGTPYYGQRFQGRVTITADKSTNTVFMELSSLRSEDTAMYFCARER
DIGSLYYFDSWGQGTLVTVSS
SEQ ID NO: 13
QVQLVQSGAEVRKPGSSVMVSCKASGGTFSTYAINWLRQAPGQGLEWMGG
IIPIFGTPYYGQRFQGRVTITADKSTNTVFMELSSLRSEDTAIYYCARDR
DSGGLYYFDSWGQGTLVTVSS
SEQ ID NO: 14
QVQLVQSGAEVKKPGSSVKVSCKTSGGTFSNYAFSWVRQAPGQGLEWMGG
IIPIFGSTNYAQSFQGRVTITADKSTSTAYMELSSLRSEDTAVYYCARDR
EMGTLYFFDQWGQGTTVTVSS
SEQ ID NO: 15
QVQLVQSGAEVKKPGASVKVSCKASGYTFTSYGISWVRQAPGQGLEWMGW
ISAYSGNTNYAQKLQGRVTMTTDTSTSTAYMELRSLRSDDTAVYYCARDG
SGWDDEDYWGQGTLVTVSS
SEQ ID NO: 16
QVQLVQSGAEVKKPGASVKVSCKASGYTFTSYGISWVRQAPGQGLEWMGW
ISAYSGNTNYAQKLQGRVTMTTDTSTSTAYMELRSLRSDDTAVYYCARGS
ILAAQMWGDIWGQGTLVTVSS
SEQ ID NO: 17
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCARER
GWDVFDIWGQGTTVTVSS
SEQ ID NO: 18
EVQLVESGGGLVQPGGSLRLSCAASGFTFSSYAMSWVRQAPGKGLEWVSS
ISGGGVSTFYADSVKGRFTISRDNSKNTLYLQMNSLRAEDTAVYYCAIVP
AAATPSGTYYWIFDLWGRGTLVTVSS
SEQ ID NO: 19
EVQLVESGGGLVQPGGSLRLSCAASGFTFSSYAMNWVRQAPGKGLEWVST
ISGSGVSTYYADSVKGRFTISRDNSKNTLYLQMNTLRAEDTAVYYCAKDR
GYDYSGSYHNWFDPWGQGTLVTVSS
SEQ ID NO: 20
QVQLVQSGAEVKKPGSSVKVSCKASGGTFSTYAISWVRQAPGQGLEWMGW
IIPIFGTGNYAQKFQGRVTITADKSTSTAYMELRSLRSEDTAVHYCARHD
YTNTVDAFDIWGQGTMVTVSS
SEQ ID NO: 21
QVQLVQSGAEVKKPGSSVKVSCKASGDTFRSYGITWVRQAPGQGLEWMGG
IIPIFGTTNYAQKFQGRVTITADKSTSTVYMELSSLRSEDTAVYYCARRR
GYSNPHWLDPWGQGTLVTVSS
SEQ ID NO: 22
QVQLVQSGAEVKKPGSSVKVSCKASGGTFSTYGILWVRQAPGQGLEWMGG
IIPIFGTANYAQKFQGRVTITADISTSTAYMELSSLRSEDTAVYYCARGG
GNYYEFVYWGQGTLVTVSS
SEQ ID NO: 23
EVQLVQSGAEVKKPGSSVKVSCKASGGTFSTYAISWVRQAPGQGLEWMGW
IIPIFDTGNYAQKIQGRVTITADKSTSTAYMELTSLRSEDTAVYYCARHD
YTNTVDAFDIWGQGTMVTVSS
SEQ ID NO: 24
EVQLVQSGAEVKKPGSSVKVSCKASGGTFSTYAISWVRQAPGQGLEWMGW
IIPIFDTGNYAQKFQGRVTITADKSTSTAYMELTSLRSEDTAVYYCARHD
YTNTVDAFDIWGQGTMVTVSS
SEQ ID NO: 25
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA
SEQ ID NO: 26
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK
SEQ ID NO: 27
QVQLQQWGAGLLKPSETLSLTCAVYGGSFSDYYWNWIRQPPGKGLEWIGE
INHRGSTNSNPSLKSRVTLSLDTSKNQFSLKLRSVTAADTAVYYCAFGYS
DYEYNWFDPWGQGTLVTVSS
SEQ ID NO: 28
EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIK
SEQ ID NO: 29
EVQLVESGGGVVQPGRSLRLSCAASGFTFSNYGMHWVRQAPGKGLEWVAV
ISYDGSTKYSADSLKGRFTISRDNSKNTLYLQMNSLRADDTAVYYCAKEG
WSFDSSGYRSWFDSWGQGTLVTVSS
LCDRs indicated according to IMGT
SEQ ID NO: 30
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIK
Light chain CDR1 IMGT
SEQ ID NO: 31
QSISSY
Light chain CDR2 IMGT
SEQ ID NO: 32
AAS
Light chain CDR3 IMGT
SEQ ID NO: 33
QQSYSTPPT
LCDRs indicated according to IMGT
SEQ ID NO: 34
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK
LCDRs indicated according to IMGT
SEQ ID NO: 35
EIVMTQSPATLSVSPGERATLSCRASQSVSSNLAWYQQKPGQAPRLLIYG
ASTRATGIPARFSGSGSGTEFTLTISSLQSEDFAVYYCQQYNNWPWTFGQ
GTKVEIK
LCDRs indicated according to IMGT
SEQ ID NO: 36
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK
LCDRs indicated according to IMGT
SEQ ID NO: 37
SYVLTQPPSVSVAPGETARITCGGDNIGRKSVYWYQQKSGQAPVLVIYYD
SDRPSGIPERFSGSNSGNTATLTISRVEAGDEADYYCQVWDGSSDHWVFG
GGTKLTVL
SEQ ID NO: 38
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTP
SEQ ID NO: 39
EIVMTQSPATLSVSPGERATLSCRASQSVSSNLAWYQQKPGQAPRLLIYG
ASTRATGIPARFSGSGSGTEFTLTISSLQSEDFAVYYCQQYNNWP
SEQ ID NO: 40
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSP
SEQ ID NO: 41
SYVLTQPPSVSVAPGETARITCGGDNIGRKSVYWYQQKSGQAPVLVIYYD
SDRPSGIPERFSGSNSGNTATLTISRVEAGDEADYYCQVWDGSSDH
SEQ ID NO: 42
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV
SEQ ID NO: 43
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
SEQ ID NO: 44
EPKSCDKTHTCPPCP
SEQ ID NO: 45
APELGRGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK
SEQ ID NO: 46
SEQ ID NO: 47
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK
SEQ ID NO: 48
SEQ ID NO: 49
SEQ ID NO: 50
GQPREPQVYTDPPSREEMTKNQVSLTCEVKGFYPSDIAVEWESNGQPENN
YKTTPPVLDSDGSFFLYSKLTVDKSRWQQGNVFSCSVMHEALHNHYTQKS
LSLSPGK
SEQ ID NO: 51
GQPREPQVYTKPPSREEMTKNQVSLKCLVKGFYPSDIAVEWESNGQPENN
YKTTPPVLDSDGSFFLYSKLTVDKSRWQQGNVFSCSVMHEALHNHYTQKS
LSLSPGK
SEQ ID NO: 52
QVQLQESGPGLVKPSETLSLTCTVSDDSISDYYWSWIRQPPGKGLEWIGY
IYYSGNTKYNPSLKNRVTISVDTSKSQFSLKLTSVTAADTAVYYCARIPL
TGDFDFWAQGTLVTVSS
SEQ ID NO: 53
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCAKER
GWDVFDLWGQGTTVTVSS
SEQ ID NO: 54
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCAKER
GWDVFDVWGQGTTVTVSS
SEQ ID NO: 55
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCAREK
GWDVFDLWGQGTTVTVSS
SEQ ID NO: 56
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCAREK
GWDVFDVWGQGTTVTVSS
SEQ ID NO: 57
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCARER
GWDIFDVWGQGTTVTVSS
SEQ ID NO: 58
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCARER
GWDLFDLWGQGTTVTVSS
SEQ ID NO: 59
QVQLVQSGGGVVQPGRSLRLSCAASGFTFSSYDTHWVRQAPGKGLEWVAV
ISYDGSNKYYADSVKGRFTISRDNSKNTLYLHMNSLRAEDTAMYYCARER
GWDLFDVWGQGTTVTVSS
SEQ ID NO: 60
SYSWS
SEQ ID NO: 61
YIDYSGSTNYNPSLKS
SEQ ID NO: 62
DLLYKWNYVEGFDI
SEQ ID NO: 63
YIDYSGTTNFNPSLKS
SEQ ID NO: 64
SYDTH
SEQ ID NO: 65
VISYDGSNKYYADSVKG
SEQ ID NO: 66
ERGWDVFDI
SEQ ID NO: 67
KYVVS
SEQ ID NO: 68
GIIPMFGTANYAQMFQG
SEQ ID NO: 69
DKAVAGLYYFDS
SEQ ID NO: 70
DYYWS
SEQ ID NO: 71
YIYYSGNTKYNPSLKN
SEQ ID NO: 72
IPLTGEFDY
SEQ ID NO: 73
SYGMH
SEQ ID NO: 74
VISYHGSDKYYADSVKG
SEQ ID NO: 75
DGDNWDVFDI
SEQ ID NO: 76
TYAIN
SEQ ID NO: 77
GIIPIFGTAYYAQEFQD
SEQ ID NO: 78
ERELGALYAFDI
SEQ ID NO: 79
SHAIS
SEQ ID NO: 80
GIIPLFDTAKNAQKFQG
SEQ ID NO: 81
DRETGTLYYFDY
SEQ ID NO: 82
TNALN
SEQ ID NO: 83
WINTHTGNPTYAQGFIG
SEQ ID NO: 84
EPNWGVYFDY
SEQ ID NO: 85
ELSMH
SEQ ID NO: 86
GSDPEHGETVDAQKFQG
SEQ ID NO: 87
GGTYYYGSGSYYTLDF
SEQ ID NO: 88
NFAFS
SEQ ID NO: 89
GIIPMFDTAKYAQKFQG
SEQ ID NO: 90
DRAIGTLYYFDY
SEQ ID NO: 91
GIIPIFGTPYYGQRFQG
SEQ ID NO: 92
ERDIGSLYYFDS
SEQ ID NO: 93
DRDSGGLYYFDS
SEQ ID NO: 94
NYAFS
SEQ ID NO: 95
GIIPIFGSTNYAQSFQG
SEQ ID NO: 96
DREMGTLYFFDQ
SEQ ID NO: 97
SYGIS
SEQ ID NO: 98
WISAYSGNTNYAQKLQG
SEQ ID NO: 99
DGSGWDDFDY
SEQ ID NO: 100
GSILAAQMWGDI
SEQ ID NO: 101
SYAMS
SEQ ID NO: 102
SISGGGVSTFYADSVKG
SEQ ID NO: 103
VPAAATPSGTYYWIFDL
SEQ ID NO: 104
SYAMN
SEQ ID NO: 105
TISGSGVSTYYADSVKG
SEQ ID NO: 106
DRGYDYSGSYHNWFDP
SEQ ID NO: 107
TYAIS
SEQ ID NO: 108
WIIPIFGTGNYAQKFQG
SEQ ID NO: 109
HDYTNTVDAFDI
SEQ ID NO: 110
SYGIT
SEQ ID NO: 111
GIIPIFGTTNYAQKFQG
SEQ ID NO: 112
RRGYSNPHWLDP
SEQ ID NO: 113
TYGIL
SEQ ID NO: 114
GIIPIFGTANYAQKFQG
SEQ ID NO: 115
GGGNYYEFVY
SEQ ID NO: 116
WIIPIFDTGNYAQKIQG
SEQ ID NO: 117
WIIPIFDTGNYAQKFQG
SEQ ID NO: 118
IPLTGDFDF
SEQ ID NO: 119
ERGWDVFDL
SEQ ID NO: 120
ERGWDVFDV
SEQ ID NO: 121
EKGWDVFDL
SEQ ID NO: 122
EKGWDVFDV
SEQ ID NO: 123
ERGWDIFDV
SEQ ID NO: 124
ERGWDLFDL
SEQ ID NO: 125
ERGWDLFDV

1. A multispecific antibody comprising a binding domain that binds to LAG-3 and a binding domain that binds to PD-L1, or a variant thereof that maintains the binding specificity of the antibody, wherein the antibody or variant has comparable, or equal or higher, potency than a combination of reference antibodies, wherein the combination of reference antibodies comprises a reference antibody comprising two heavy chain variable regions having an amino acid sequence as set forth in SEQ ID NO: 27 and two light chain variable regions having an amino acid sequence as set forth in SEQ ID NO: 28, and a reference antibody comprising two heavy chain variable regions having an amino acid sequence as set forth in SEQ ID NO: 25 and two light chain variable regions having an amino acid sequence as set forth in SEQ ID NO: 26.

2. The multispecific antibody or variant thereof according to claim 1, wherein the antibody or variant comprises a single binding domain that binds to LAG-3 and/or a single binding domain that binds to PD-L1.

3. The multispecific antibody or variant thereof according to claim 1, wherein the potency is determined by measuring tumor volume reduction in an in vivo mouse study, in particular wherein a comparable potency is a tumor volume reduction within a 5 fold range of the tumor volume reduction of the combination of reference antibodies, and includes a 5 to 2 fold, preferably a 3 to 2 fold, deviation from the tumor volume reduction of the combination of reference antibodies.

4. (canceled)

5. The multispecific antibody or variant thereof according to claim 1, wherein the potency is determined by the potency in blocking ligand or receptor binding to LAG-3 and/or PD-L1, in particular wherein the potency in blocking ligand binding to LAG-3 and/or PD-L1 is measured in a PD-1/LAG-3 reporter assay.

6. (canceled)

7. The multispecific antibody or variant thereof according to claim 5, wherein a comparable potency is a potency in blocking ligand or receptor binding to LAG-3 and/or PD-L1, within a 5 fold range of the potency in blocking ligand or receptor binding to LAG-3 and/or PD-L1 of the combination of reference antibodies, and includes a 5 to 2 fold, at least a 3 to 2 fold, deviation from the LAG-3 and/or PD-L1 blocking activity of the combination of reference antibodies.

8. The multispecific antibody or variant thereof according to claim 1, wherein the binding domain that binds to LAG-3 comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 1;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 2;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 3;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 4;
e) heavy chain CDR1 (HCDR1), heavy chain CDR2 (HCDR2), and heavy chain CDR3 (HCDR3), of a heavy chain variable region having an amino acid sequence as set forth in SEQ ID NO: 5;
f) heavy chain CDR1 (HCDR1), heavy chain CDR2 (HCDR2), and heavy chain CDR3 (HCDR3), of a heavy chain variable region having an amino acid sequence as set forth in SEQ ID NO: 6;
g) heavy chain CDR1 (HCDR1), heavy chain CDR2 (HCDR2), and heavy chain CDR3 (HCDR3), of a heavy chain variable region having an amino acid sequence as set forth in SEQ ID NO: 7;
h) heavy chain CDR1 (HCDR1), heavy chain CDR2 (HCDR2), and heavy chain CDR3 (HCDR3), of a heavy chain variable region having an amino acid sequence as set forth in SEQ ID NO: 8;
i) heavy chain CDR1 (HCDR1), heavy chain CDR2 (HCDR2), and heavy chain CDR3 (HCDR3), of a heavy chain variable region having an amino acid sequence as set forth in SEQ ID NO: 9;
j) heavy chain CDR1 (HCDR1), heavy chain CDR2 (HCDR2), and heavy chain CDR3 (HCDR3), of a heavy chain variable region having an amino acid sequence as set forth in SEQ ID NO: 10;
k) heavy chain CDR1 (HCDR1), heavy chain CDR2 (HCDR2), and heavy chain CDR3 (HCDR3), of a heavy chain variable region having an amino acid sequence as set forth in SEQ ID NO: 11;
l) heavy chain CDR1 (HCDR1), heavy chain CDR2 (HCDR2), and heavy chain CDR3 (HCDR3), of a heavy chain variable region having an amino acid sequence as set forth in SEQ ID NO: 12;
m) heavy chain CDR1 (HCDR1), heavy chain CDR2 (HCDR2), and heavy chain CDR3 (HCDR3), of a heavy chain variable region having an amino acid sequence as set forth in SEQ ID NO: 13;
n) heavy chain CDR1 (HCDR1), heavy chain CDR2 (HCDR2), and heavy chain CDR3 (HCDR3), of a heavy chain variable region having an amino acid sequence as set forth in SEQ ID NO: 14;
o) heavy chain CDR1 (HCDR1), heavy chain CDR2 (HCDR2), and heavy chain CDR3 (HCDR3), of a heavy chain variable region having an amino acid sequence as set forth in SEQ ID NO: 15;
p) heavy chain CDR1 (HCDR1), heavy chain CDR2 (HCDR2), and heavy chain CDR3 (HCDR3), of a heavy chain variable region having an amino acid sequence as set forth in SEQ ID NO: 16;
q) heavy chain CDR1 (HCDR1), heavy chain CDR2 (HCDR2), and heavy chain CDR3 (HCDR3), of a heavy chain variable region having an amino acid sequence as set forth in SEQ ID NO: 17,
r) heavy chain CDR1 (HCDR1), heavy chain CDR2 (HCDR2), and heavy chain CDR3 (HCDR3), of a heavy chain variable region having an amino acid sequence as set forth in SEQ ID NO: 52,
s) heavy chain CDR1 (HCDR1), heavy chain CDR2 (HCDR2), and heavy chain CDR3 (HCDR3), of a heavy chain variable region having an amino acid sequence as set forth in SEQ ID NO: 53,
t) heavy chain CDR1 (HCDR1), heavy chain CDR2 (HCDR2), and heavy chain CDR3 (HCDR3), of a heavy chain variable region having an amino acid sequence as set forth in SEQ ID NO: 54,
u) heavy chain CDR1 (HCDR1), heavy chain CDR2 (HCDR2), and heavy chain CDR3 (HCDR3), of a heavy chain variable region having an amino acid sequence as set forth in SEQ ID NO: 55,
v) heavy chain CDR1 (HCDR1), heavy chain CDR2 (HCDR2), and heavy chain CDR3 (HCDR3), of a heavy chain variable region having an amino acid sequence as set forth in SEQ ID NO: 56,
w) heavy chain CDR1 (HCDR1), heavy chain CDR2 (HCDR2), and heavy chain CDR3 (HCDR3), of a heavy chain variable region having an amino acid sequence as set forth in SEQ ID NO: 57,
x) heavy chain CDR1 (HCDR1), heavy chain CDR2 (HCDR2), and heavy chain CDR3 (HCDR3), of a heavy chain variable region having an amino acid sequence as set forth in SEQ ID NO: 58, or
y) heavy chain CDR1 (HCDR1), heavy chain CDR2 (HCDR2), and heavy chain CDR3 (HCDR3), of a heavy chain variable region having an amino acid sequence as set forth in SEQ ID NO: 59,
wherein each of the HCDRs may comprise at most three, two, or one amino acid substitution.

9. The multispecific antibody or variant thereof according to claim 1, wherein the LAG-3 binding domain comprises a heavy chain variable region having an amino acid sequence as set forth in any one of SEQ ID NO: 1-17 or SEQ ID NO: 52-59, or having at least 80%, at least 85%, at least 90%, or at least 95% sequence identity thereto.

10. (canceled)

11. (canceled)

12. The multispecific antibody or variant thereof according to claim 1, wherein the binding domain that binds to PD-L1 comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 20;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 21;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 22;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 23; or
e) heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein each of the HCDRs may comprise at most three, two, or one amino acid substitution.

13. The multispecific antibody or variant thereof according to claim 1, wherein the PD-L1 binding domain comprises a heavy chain variable region having an amino acid sequence as set forth in any one of SEQ ID NO: 20-24 or having at least 80%, at least 85%, at least 90%, or at least 95% sequence identity thereto.

14. (canceled)

15. (canceled)

16. A multispecific antibody, or variant thereof that maintains the binding specificity of the antibody, wherein the antibody or variant thereof comprises a binding domain that specifically binds to an extracellular domain of LAG-3 and a binding domain that specifically binds to an extracellular domain of a protein of the B7 family,
wherein the LAG-3 binding domain comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 1;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 2;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 3;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 4;
e) heavy chain CDR1 (HCDR1), heavy chain CDR2 (HCDR2), and heavy chain CDR3 (HCDR3), of a heavy chain variable region having an amino acid sequence as set forth in SEQ ID NO: 5;
f) heavy chain CDR1 (HCDR1), heavy chain CDR2 (HCDR2), and heavy chain CDR3 (HCDR3), of a heavy chain variable region having an amino acid sequence as set forth in SEQ ID NO: 6;
g) heavy chain CDR1 (HCDR1), heavy chain CDR2 (HCDR2), and heavy chain CDR3 (HCDR3), of a heavy chain variable region having an amino acid sequence as set forth in SEQ ID NO: 7;
h) heavy chain CDR1 (HCDR1), heavy chain CDR2 (HCDR2), and heavy chain CDR3 (HCDR3), of a heavy chain variable region having an amino acid sequence as set forth in SEQ ID NO: 8;
i) heavy chain CDR1 (HCDR1), heavy chain CDR2 (HCDR2), and heavy chain CDR3 (HCDR3), of a heavy chain variable region having an amino acid sequence as set forth in SEQ ID NO: 9;
j) heavy chain CDR1 (HCDR1), heavy chain CDR2 (HCDR2), and heavy chain CDR3 (HCDR3), of a heavy chain variable region having an amino acid sequence as set forth in SEQ ID NO: 10;
k) heavy chain CDR1 (HCDR1), heavy chain CDR2 (HCDR2), and heavy chain CDR3 (HCDR3), of a heavy chain variable region having an amino acid sequence as set forth in SEQ ID NO: 11;
l) heavy chain CDR1 (HCDR1), heavy chain CDR2 (HCDR2), and heavy chain CDR3 (HCDR3), of a heavy chain variable region having an amino acid sequence as set forth in SEQ ID NO: 12;
m) heavy chain CDR1 (HCDR1), heavy chain CDR2 (HCDR2), and heavy chain CDR3 (HCDR3), of a heavy chain variable region having an amino acid sequence as set forth in SEQ ID NO: 13;
n) heavy chain CDR1 (HCDR1), heavy chain CDR2 (HCDR2), and heavy chain CDR3 (HCDR3), of a heavy chain variable region having an amino acid sequence as set forth in SEQ ID NO: 14;
o) heavy chain CDR1 (HCDR1), heavy chain CDR2 (HCDR2), and heavy chain CDR3 (HCDR3), of a heavy chain variable region having an amino acid sequence as set forth in SEQ ID NO: 15;
p) heavy chain CDR1 (HCDR1), heavy chain CDR2 (HCDR2), and heavy chain CDR3 (HCDR3), of a heavy chain variable region having an amino acid sequence as set forth in SEQ ID NO: 16;
q) heavy chain CDR1 (HCDR1), heavy chain CDR2 (HCDR2), and heavy chain CDR3 (HCDR3), of a heavy chain variable region having an amino acid sequence as set forth in SEQ ID NO: 17,
r) heavy chain CDR1 (HCDR1), heavy chain CDR2 (HCDR2), and heavy chain CDR3 (HCDR3), of a heavy chain variable region having an amino acid sequence as set forth in SEQ ID NO: 52,
s) heavy chain CDR1 (HCDR1), heavy chain CDR2 (HCDR2), and heavy chain CDR3 (HCDR3), of a heavy chain variable region having an amino acid sequence as set forth in SEQ ID NO: 53,
t) heavy chain CDR1 (HCDR1), heavy chain CDR2 (HCDR2), and heavy chain CDR3 (HCDR3), of a heavy chain variable region having an amino acid sequence as set forth in SEQ ID NO: 54,
u) heavy chain CDR1 (HCDR1), heavy chain CDR2 (HCDR2), and heavy chain CDR3 (HCDR3), of a heavy chain variable region having an amino acid sequence as set forth in SEQ ID NO: 55,
v) heavy chain CDR1 (HCDR1), heavy chain CDR2 (HCDR2), and heavy chain CDR3 (HCDR3), of a heavy chain variable region having an amino acid sequence as set forth in SEQ ID NO: 56,
w) heavy chain CDR1 (HCDR1), heavy chain CDR2 (HCDR2), and heavy chain CDR3 (HCDR3), of a heavy chain variable region having an amino acid sequence as set forth in SEQ ID NO: 57,
x) heavy chain CDR1 (HCDR1), heavy chain CDR2 (HCDR2), and heavy chain CDR3 (HCDR3), of a heavy chain variable region having an amino acid sequence as set forth in SEQ ID NO: 58, or
y) heavy chain CDR1 (HCDR1), heavy chain CDR2 (HCDR2), and heavy chain CDR3 (HCDR3), of a heavy chain variable region having an amino acid sequence as set forth in SEQ ID NO: 59,
wherein each of the HCDRs may comprise at most three, two, or one amino acid substitution.

17. The multispecific antibody or variant thereof according to claim 16, wherein the LAG-3 binding domain comprises a heavy chain variable region having an amino acid sequence as set forth in any one of SEQ ID NO: 1-17 or SEQ ID NO: 52-59, or having at least 80%, at least 85%, at least 90%, or at least 95% sequence identity thereto.

18. The multispecific antibody or variant thereof according to claim 16, wherein the LAG-3 binding domain further comprises a light chain variable region, in particular wherein the LAG-3 binding domain further comprises a CH1 and CL region.

19. (canceled)

20. The multispecific antibody or variant thereof according to claim 16, wherein the antibody or variant is monovalent for binding to LAG-3.

21. The multispecific antibody or variant thereof according claim 16, wherein the protein of the B7 family is selected from the group consisting of PD-L1, PD-L2, CD80, CD86, B7-H4, TNFRSF14, and B7-H7, in particular wherein the protein of the B7 family is PD-L1.

22. (canceled)

23. The multispecific antibody or variant thereof according to claim 16, wherein the variable domain that specifically binds to an extracellular domain of a protein of the B7 family blocks the binding of PD-L1 to its receptor, in particular wherein the receptor is PD-1 and/or CD80.

24. (canceled)

25. The multispecific antibody or variant thereof according to claim 16, wherein the binding domain that binds to PD-L1 comprises a heavy chain variable region comprising:
a) heavy chain CDR1 (HCDR1), heavy chain CDR2 (HCDR2), and heavy chain CDR3 (HCDR3), of a heavy chain variable region having an amino acid sequence as set forth in SEQ ID NO: 20;
b) heavy chain CDR1 (HCDR1), heavy chain CDR2 (HCDR2), and heavy chain CDR3 (HCDR3), of a heavy chain variable region having an amino acid sequence as set forth in SEQ ID NO: 21;
c) heavy chain CDR1 (HCDR1), heavy chain CDR2 (HCDR2), and heavy chain CDR3 (HCDR3), of a heavy chain variable region having an amino acid sequence as set forth in SEQ ID NO: 22;
d) heavy chain CDR1 (HCDR1), heavy chain CDR2 (HCDR2), and heavy chain CDR3 (HCDR3), of a heavy chain variable region having an amino acid sequence as set forth in SEQ ID NO: 23; or
e) heavy chain CDR1 (HCDR1), heavy chain CDR2 (HCDR2), and heavy chain CDR3 (HCDR3), of a heavy chain variable region having an amino acid sequence as set forth in SEQ ID NO: 24,
wherein each of the HCDRs may comprise at most three, two, or one amino acid substitution.

26. The multispecific antibody or variant thereof according to claim 16, wherein the PD-L1 binding domain comprises a heavy chain variable region having an amino acid sequence as set forth in any one of SEQ ID NO: 20-24, or having at least 80%, at least 85%, at least 90%, or at least 95% sequence identity thereto.

27. The multispecific antibody or variant thereof according to claim 16, wherein the PD-L1 binding domain further comprises a light chain variable region, in particular wherein the PD-L1 binding domain further comprises a CH1 and CL region.

28. (canceled)

29. The multispecific antibody or variant thereof according to claim 16, wherein the antibody or variant is monovalent for binding to PD-L1.

30. The multispecific antibody or variant thereof according to claim 16, wherein the antibody or variant has comparable, or equal or higher, potency than a combination of reference antibodies, wherein the combination of reference antibodies comprises a reference antibody comprising two heavy chain variable regions having an amino acid sequence as set forth in SEQ ID NO: 27 and two light chain variable regions having an amino acid sequence as set forth in SEQ ID NO: 28, and a reference antibody comprising two heavy chain variable regions having an amino acid sequence as set forth in SEQ ID NO: 25 and two light chain variable regions having an amino acid sequence as set forth in SEQ ID NO: 26.

31. The multispecific antibody or variant thereof according to claim 30, wherein the potency is determined by measuring tumor volume reduction in an in vivo mouse study.

32. The multispecific antibody or variant thereof according to claim 30, wherein a comparable potency includes a 5 to 2 fold, at least a 3 to 2 fold, deviation from the tumor volume reduction of the combination of reference antibodies.

33. The multispecific antibody or variant thereof according to claim 30, wherein the potency is determined by the potency in blocking ligand or receptor binding to LAG-3 and/or PD-L1, in particular wherein the potency in blocking ligand binding to LAG-3 and/or PD-L1 is measured in a PD-1/LAG-3 reporter assay.

34. (canceled)

35. The multispecific antibody or variant thereof according to claim 33, wherein a comparable potency is a potency in blocking ligand or receptor binding to LAG-3 and/or PD-L1, within a 5 fold range of the potency in blocking ligand or receptor binding to LAG-3 and/or PD-L1 of the combination of reference antibodies, and includes a 5 to 2 fold, at least 3 to 2 fold, deviation from the LAG-3 and PD-L1 blocking activity of the combination of reference antibodies.

36. The multispecific antibody or variant thereof according to claim 1, wherein the antibody or variant is a full length antibody, in particular a full length bispecific antibody.

37. A pharmaceutical composition comprising the multispecific antibody or variant thereof according to claim 1 and a pharmaceutical acceptable carrier, diluent or excipient.

38. (canceled)

39. (canceled)

40. (canceled)

41. A method for treating a disease, comprising administering an effective amount of the multispecific antibody or variant thereof as claimed in claim 1.

42. A method for treating a disease associated with a suppressed immune system, in particular cancer, comprising administering an effective amount of the multispecific antibody or variant thereof as claimed in claim 1.

43. A method for treating cancer, comprising administering an effective amount of the multispecific antibody or variant thereof as claimed in claim 1.

44. A vector comprising a nucleic acid sequence encoding the heavy chain variable region of a LAG-3 binding domain as defined in claim 16 and a nucleic acid sequence encoding the heavy chain variable region of a PD-L1 binding domain.

45. The vector according to claim 44, wherein the vector further comprises a nucleic acid sequence encoding a CH1 region and at least a hinge, CH2 and CH3 region.

46. The vector according to claim 44, further comprising at least one nucleic acid sequence encoding a light chain variable region, and at least a CL region.

47. The vector according to claim 46, wherein the light chain variable region is a light chain variable region of a light chain that is capable of pairing with multiple heavy chains having different epitope specificities.

48. A cell comprising a nucleic acid sequence encoding the heavy chain variable region of a LAG-3 binding domain as defined in claim 16 and a nucleic acid sequence encoding the heavy chain variable region of a PD-L1 binding domain.

49. The cell according to claim 48, wherein the cell further comprises a nucleic acid sequence encoding a CH1 region and at least a hinge, CH2 and CH3 region.

50. The cell according to claim 48, further comprising at least one nucleic acid sequence encoding a light chain variable region, and at least a CL region.

51. The cell according to claim 50, wherein the light chain variable region is a light chain variable region of a light chain that is capable of pairing with multiple heavy chains having different epitope specificities.

52. A cell producing a multispecific antibody or variant thereof according to claim 1, in particular wherein the cell is a recombinant cell, which has been transformed with a vector.

53. (canceled)
